(19) United States
(12) Patent Application Publication
(10) Pub. No.: US 2018/0028468 A1
(43) Pub. Date: Feb. 1, 2018

(54) SUSTAINED RELEASE DRUG DELIVERY DEVICES

(71) Applicant: OCUNEXUS THERAPEUTICS, INC., San Diego, CA (US)

(72) Inventors: David L. BECKER, Hertfordshire (GB); Anthony R.J. PHILLIPS, San Diego, CA (US); Daniel J. GILMARTIN, London (GB)

(21) Appl. No.: 15/583,882

(22) Filed: May 1, 2017

Related U.S. Application Data
(60) Provisional application No. 62/330,050, filed on Apr. 29, 2016.

(51) Int. Cl. A61K 9/70; A61K 31/713; D06M 15/507; A61K 47/42; A61K 47/36; A61K 47/34; C12N 15/113; A61K 9/00

(52) U.S. Cl. CPC A61K 9/7092; C12N 15/1138; A61K 31/713; A61K 9/0014; A61K 47/42; A61K 47/36; A61K 47/34; D06M 15/507; C12N 2310/11; C12N 2320/31; C12N 2320/35; C12N 2320/32; D06M 2101/14

(57) ABSTRACT

Sustained release drug delivery devices including scaffolds comprising combinations of anti-connexin, anti-gap junction, anti-hemichannel and/or other agents, for example, anti-connexin 26 and anti-connexin 43 polynucleotides or peptidomimetics, uses therefor, and kits comprising sustained release scaffold.

### RELATED APPLICATION

This application claims priority to U.S. Provisional Application Ser. No. 62/330,050, entitled SUSTAINED RELEASE DRUG DELIVERY DEVICES, filed Apr. 29, 2016, the contents of which are herein incorporated by reference as if set forth in their entirety.

### FIELD

The inventions relate to devices for sustained release of compounds for controlling gap junction protein channel activities.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

All U.S. patents, U.S. patent application publications, foreign patents, foreign and PCT published applications, articles and other documents, references and publications noted herein, and all those that are published as a part of any patent or patents that issue herefrom, are hereby incorporated by reference in their entirety.

Wound injury in humans and other mammals triggers an organized complex cascade of cellular and biochemical events that, in most cases, will result in a healed wound. Every wound goes through a continuous repair and healing process that normally takes several weeks. It is a dynamic process that proceeds via a complex process encompassing four precisely and highly programmed phases: hemostasis, inflammation, proliferation, and remodeling. For a wound to heal successfully, all four phases must occur in the proper sequence and time frame. Interruptions, aberrancies, or prolongation in any phase in the process can lead to impaired or delayed wound healing, or a chronic wound.

Gap junctions are cell membrane structures that facilitate direct cell-cell communication. They directly connect the cytoplasm of two cells, which allows various molecules, ions, and electrical impulses to directly pass between cells. Gap junction channels are found throughout the body and are expressed in virtually all tissues of the body, with the exception of mature skeletal muscle and mobile cell types such as sperm and erythrocytes. A gap junction channel is formed of two half-channels, called “connexons” or “hemichannels,” with a hemichannel in one cell membrane docking with another hemichannel in an opposing membrane to form a single gap junction channel. Each gap junction hemichannels is composed of six connexin protein subunits.

In general, connexins are a family of proteins, and at least 20 human and 19 murine isoforms have been identified. Different tissues and cell types are reported to have characteristic patterns of connexin protein expression and tissues have been shown to alter connexin protein expression pattern following injury or transplantation (Qiu, C. et al., (2003) Current Biology, 13:1967-1703; Brandner et al., (2004), J. Invest Dermatol. 122:1310-20). Each connexin is named based on its calculated molecular weight. Thus, connexin 43 and connexin 26 are 43 kD and 26 kD proteins, respectively.

It has been reported that abnormal connexin function may be linked to certain disease states, and mutations in connexin genes are reported cause a variety of disorders such as myelin-related diseases, skin disorders, hearing loss, congenital cataract, or more complex syndromes such as the oculodendrodigital dysplasia. For example, defects in the connexin 26 gene are said to lead to DFNB1 (also known as connexin 26 deafness), the most common form of congenital deafness in developed countries. See Pfenninger et al., Mutations in connexin genes and disease Eur. J. Clin. Invest. 41:103-116 (2011).

Antisense technology has been proposed for the modulation of the expression for genes implicated in viral, fungal, and metabolic diseases. See, for example, U.S. Pat. No. 5,166,195, (oligonucleotide inhibitors of HIV) and U.S. Pat. No. 5,004,810 (oligomers for hybridizing to herpes simplex virus Vmw65 mRNA and inhibiting replication). See also U.S. Pat. No. 7,098,190 issued to Becker and Green (“Formulations comprising antisense nucleotides to connexins”). Peptide inhibitors of gap junctions and hemichannels have also been reported. See, e.g., Berthoud, V. M. et al., Am J. Physiol. Lung Cell Mol. Physiol. 279:L619-L622 (2000); Evans, W. H. and Boitano, S. Biochem. Soc. Trans. 29:606-612, and De Vriese A. S., et al. Kidney Int. 61:177-185 (2001). See also Becker and Green PCT/US06/04131 (“Anti-connexin compounds and uses thereof”).

### BRIEF SUMMARY

The inventions described and claimed herein have many attributes, aspects, and embodiments including, but not limited to, those set forth or described or referenced in this Brief Summary. It is not intended to be all-inclusive and the inventions described and claimed herein are not limited to or by the aspects, features or embodiments identified in this Brief Summary, which is included for purposes of illustration only and not restriction.

In one aspect, the inventions relate to devices for sustained release of compounds for controlling gap junction protein channel activities. In another aspect, the inventions relate to devices for sustained release of compounds for the management of impaired healing wounds including, for example, chronic wounds.

In one aspect the present disclosure features a sustained release drug delivery device of a degradable scaffold comprising one or more anti-connexin agents. Anti-connexin agents associated with scaffolds may include, for example, anti-connexin polynucleotides, e.g. those that target connexin encoding mRNA species, anti-connexin peptides or peptidomimetics, gap junction closing compounds, and/or hemichannel closing or blocking compounds, for the treatment of wounds, including acute, slow to heal, impaired healing, delayed healing, and chronic wounds, for example. In some aspects the anti-connexin agent may be, for example, an anti-connexin 43, anti-connexin 37, anti-connexin 30, anti-connexin 31.1 or anti-connexin 32 agent. They include, for example, connexin 26 and/or connexin 43 peptides or peptidomimetics, peptides or peptidomimetics comprising connexin extracellular domains, for example. They also include connexin carboxy-terminal peptides, such as connexin 43 carboxy-terminal peptides that bind to the ZO-1 binding site, for example.

This disclosure relates in some aspects to sustained release drug delivery devices suitable for sustained administration of an anti-connexin agent, comprising a scaffold core and at least one polymer coating. In some aspects the polymer coating can be selected from (a) a polymer coating comprising an anti-connexin agent and a biodegradable and biocompatible polymer and (b) a coating comprising a biodegradable polyester and an anti-connexin modulating agent. In some aspects, the sustained release drug delivery device may comprise two or more coatings comprising an anti-connexin agent and a biodegradable and biocompatible copolymer and/or two or more coatings comprising a biodegradable polyester and an anti-connexin agent. In some aspects, each coating may comprise one or more layers. In some aspects, the coating can comprise one or a plurality of polymer coatings on the scaffold comprising one or a plurality of gap junction modulators and one or a plurality of biodegradable polymers, with the coating able to release one or a plurality of gap junction modulators into the skin tissue.

In some aspects the sustained release drug delivery device may comprise at least one copolymer coating comprising an anti-connexin agent and a biodegradable and biocompatible copolymer and at least one coating comprising a biodegradable polyester and an anti-connexin modulating agent. In some aspects, the sustained release drug delivery device may have an inner coating comprising a biodegradable polyester and an anti-connexin modulating agent and an outer coating comprising an anti-connexin agent and a biodegradable and biocompatible copolymer.

The scaffold core may, in some aspects, comprise a connective tissue blended with a biodegradable polymer. In some aspects, the connective tissue may comprise one or more of the following connective tissues: collagen, elastin, and chondroitin-4-sulfate. In some aspects, the connective tissue may be present at an amount about 50-99% collagen (w/w). In some aspects, the biodegradable polymer may comprise a biodegradable polyester polymer. In some aspects, the biodegradable polyester polymer may include or exclude: poly(L-lactide), poly(glycolide), poly(DL-lactide), poly(dioxanone), poly(DL-lactide-co-L-lactide), poly(DL-lactide-co-glycolide), poly(glycolide-co-trimethylene carbonate), poly(caprolactone) (“polycaprolactone”), poly(lactic-co-glycolic acid (PLGA), poly(dioxanone), poly(glycolide-co-trimethylene carbonate), and mixtures of any of the foregoing. In some aspects, the polyester polymer may comprise polycaprolactone (PCL). In some aspects, the amount of polycaprolactone may be present at an amount about 1-50% polycaprolactone (w/w). In some aspects, the molecular weight of the polycaprolactone may range from 10,000 Da to 3,000,000 Da. In some aspects, the amount of polycaprolactone may be present at an amount that is at least about 50% polycaprolactone (w/w).

In some aspects, the collagen and the polymer, for example, comprising the scaffold may be electrospun from a precursor material into fibers to create a scaffold sheet, from which the desired scaffold shape may be obtained. In some aspects, the precursor material can be a connective tissue. In some aspects, the shape obtained from the scaffold sheet may extracted from the sheet. The shape extraction can be, for example, a disk cut from the sheet. The extraction, for example, can be performed by cutting with a laser or punched out using a biopsy or other punch. In some aspects, by controlling fiber orientation during deposition, a three-dimensional scaffold may be fabricated having a desired shape other than a disk. Three-dimensional (3D) printing may also be used to obtain a scaffold core for the sustained release drug delivery devices of this invention. In some aspects, polymer sheets may be prepared, for example, by dip-coating onto a substrate, spin-casting a substrate, or spray-dried onto a substrate, followed by delamination of the sheet from the substrate.

In some aspects, the scaffold may be coated with a mixture comprising the anti-connexin agent. The mixture may also comprise a polymer which temporarily binds the anti-connexin agent to the scaffold (“binding polymer”, or “eluting polymer”). Such polymers may include or exclude: poly(L-lactide), poly(glycolide), poly(DL-lactide), poly(dioxanone), poly(DL-lactide-co-L-lactide), poly(DL-lactide-co-glycolide), poly(glycolide-co-trimethylene carbonate), poly(caprolactone) (“polycaprolactone”), poly(lactic-co-glycolic acid (PLGA), poly(dioxanone), poly(glycolide-co-trimethylene carbonate), and mixtures of any of the foregoing. Multiple coatings may be applied to the scaffold to achieve a desired elution profile of the anti-connexin agent. In some aspects, there can be two or more coatings on the scaffold. There can be two, three, four, or more coatings on the scaffold. In some aspects, there can be two coatings of polycaprolactone blended with a first gap junction modulator and two coatings of PLGA blended with a second gap function modulator, e.g., an anti-connexin agent. The first and second gap junction modulators can be the same type of gap junction modulator or different types of gap junction modulators. In some aspects, the polycaprolactone and PLGA polymer coatings can be alternating. In some aspects when there are multiple polymer coatings, the coatings can be alternating of the same type of polymer coatings or alternating of different types of polymer coatings.

In some aspects, each coating layer may be applied by immersing the scaffold in a solution of the dissolved binding polymer and anti-connexin agent. The scaffold may then be removed and freeze-dried (lyophilized) to remove the solvent. In one aspect, the scaffold may be removed and subject to quick evaporation by placing the scaffold in a vacuum chamber. In some aspects, the scaffold can be dried by the passage of a gas to remove the polymer solvent. The scaffold comprising the first binding polymer layer may be subsequently immersed into another anti-connexin agent solution comprising the same binding polymer as used in the prior immersion, or a different binding polymer, and the same anti-connexin agent as used in the prior immersion, or a different anti-connexin agent than that used in a prior immersion. The steps of immersion and lyophilization may be repeated up to twenty times to create one or more layers of coating and/or one or more coatings on the scaffold.

The polymer which temporarily binds the anti-connexin agent to the scaffold may comprise any polymer suitable for drug-elution. Such polymers exhibit the following properties: are non-toxic (proportional to their beneficial effect), are metabolized directly or whose hydrolysis products are metabolized, and may easily be sterilized. A review of such applicable materials may be found in J. C. Middleton, A. J. Tipton, Biomaterials, 21 (2000), 2335-2346.

In some aspects, the gap junction modulator in the biodegradable polymer is a connexin modulator. The connexin modulator can be an antisense polynucleotide that is antisense to a connexin selected from: connexin 26 (Cx26) and/or connexin 43 (Cx43).

Other connexin targets include connexin 30, connexin 30.3, connexin 31, connexin 31.1, connexin 32, connexin 37, connexin 40 and connexin 45. Other compounds that may be included in the biodegradable polymer, with or without an anti-connexin agent, are modulators of cadherin and/or catenin. The modulator can be an antisense polynucleotide against cadherin or beta-catenin, for example.

Such sustained release drug delivery devices of one or more anti-connexin, anti-cadherin and/or anti-catenin compounds may be targeted for delivery to the wound site at, on or in the skin. The one or more compounds can target the wound site by virtue of their regional proximity after being released from the degradable scaffold. The scaffold can be placed at or in the skin, and as the scaffold degrades, the one or more anti-connexin wound healing compounds in the scaffold, by way of example, can be released to the wound site at, on or in the skin.

This disclosure relates in some aspects to the use a sustained release drug delivery device comprising one or more anti-connexin 26 polynucleotides or anti-connexin 43 polynucleotides, alone or in combination with one or more other wound healing compounds, including anti-connexin polypeptides that target other connexin encoding mRNA species, anti-connexin peptides or peptidomimetics (for example, connexin 26 or connexin 43 peptides or peptidomimetics, peptides or peptidomimetics comprising connexin extracellular domains and connexin carboxy-terminal peptides), gap junction closing compounds, and/or hemichannel closing compounds, and others noted herein, e.g., anti-cadherin and/or anti-catenin compounds, for the treatment of wounds, including acute, delayed healing and chronic wounds. In some aspects, the anti-connexin 26 polynucleotides can be combined with anti-connexin 43 polynucleotides.

In some aspects, the antisense connexin polynucleotide can comprise a synthetic anti-Cx26 polynucleotide or a synthetic anti-Cx43 polynucleotide or a mixture comprising both, wherein the polynucleotide(s) comprises at least about 10, 15, 20, 25, 30, 35, 40, 45, 50, 55, 60, 65, 70, 75, 80, 85, 90, or 95 nucleotide residues. Any of the polynucleotides can further comprise a modified or unmodified phosphodiester backbone. The modified backbones can further comprise one or more internucleotide linkages selected from the group consisting of phosphorothioate, methylphosphorate, and locked nucleic acid linkages. Sugars and nucleotides themselves can also be modified using methods know in the art.

In some aspects, the antisense connexin polynucleotide can comprise a nucleotide sequence selected from the group consisting of: TGTATTGGGACAAGGCCAGG (SEQ ID NO: 1), or ATCTCTTCGATGTCCTTAAA (SEQ ID NO: 2), or a nucleotide sequence that has at least about 65%, 70%, 75%, 80%, 85%, 90%, 91%, 92%, 93%, 94%, 95%, 96%, 97%, 98%, or 99% sequence identity with a nucleotide of SEQ ID NOS: 1-2, above.

In one aspect, the sustained release drug delivery device can comprise an anti-Cx26 polynucleotide and a carrier, optionally a pharmaceutically acceptable carrier, wherein the sustained release drug delivery device optionally comprises from about 0.1 to about 1000 micrograms of the anti-Cx26 polynucleotide. In some aspects, the sustained release drug delivery device can comprise an anti-Cx43 polynucleotide and a carrier, optionally a pharmaceutically acceptable carrier, wherein the sustained release drug delivery device optionally comprises from about 100 to about 1000 or 50,000 micrograms of the anti-Cx43 polynucleotide. The compounds described herein may be calculated to be present at amounts ranging from about 100 to about 1000 or 50,000 micrograms per one square millimeter to one square centimeter of scaffold. They can also be calculated to be present at amounts ranging from about 1 to about 50 mg of coating substance, for example, from 1 or 3 mg/mL to 3-10 or 10-30 mg/mL, and up to 50 to 100 mg/mL and may be calculated by using the total volume of coating material or by the volume of one or more layers of each coating substance.

This disclosure relates in some aspects to a sustained release drug delivery device comprising an anti-connexin 26 polynucleotide as a first anti-connexin agent in combination with a second anti-connexin agent, preferably an anti-connexin 43 polynucleotide. Treatment of a subject, e.g., for a wound, with sustained release drug delivery devices described in this disclosure comprising a first anti-connexin agent and a second anti-connexin agent, may comprise the simultaneous, separate, sequential or sustained administration of the first anti-connexin agent and a second anti-connexin agent.

In some aspects, the second anti-connexin agent may be selected from the group consisting of anti-connexin oligonucleotides, anti-connexin peptides or anti-connexin peptidomimetics (for example, anti-connexin peptides or peptidomimetics comprising connexin extracellular domains and/or connexin carboxy-terminal peptides), gap junction closing compounds, hemichannel closing compounds, and/or other gap junction modulating agents useful for wound healing, provided that the second anti-connexin agent is a different chemical entity as compared to the first anti-connexin agent. Preferred second anti-connexin agents include anti-connexin 43 oligonucleotides (ODN). Preferred peptides or peptidomimetics, are anti-connexin 43 peptides or peptidomimetics, e.g., anti-connexin 43 hemichannel blocking peptides or anti-connexin 43 hemichannel blocking peptidomimetics. Preferred gap junction closing compounds and hemichannel closing compounds are connexin 43 gap junction closing compounds and connexin 43 hemichannel closing compounds. Preferred connexin carboxy-terminal peptides are connexin 43 carboxy-terminal peptides.

In some aspects, this disclosure provides sustained release drug delivery devices comprising (a) an anti-connexin peptide or peptidomimetic and (b) an antisense polynucleotide to the mRNA of a connexin protein. In some aspects, the connexin can be connexin 26 or connexin 43. Most preferably, this connexin is connexin 26. In some aspects, this disclosure provides sustained release drug delivery devices comprising (a) and/or (b) and one or more of a gap junction closing compound, a hemichannel closing compound, and a connexin carboxy-terminal polypeptide useful for wound healing. Most preferably, in the case of gap junction closing compound and hemichannel closing compounds useful for wound healing the gap junction or hemichannel is a combination of a connexin 26 gap junction or hemichannel and a connexin 43 gap junction or hemichannel. Most preferably, in the case of connexin carboxy-terminal polypeptides useful for wound healing, the connexin is connexin 26.

In some aspects, this disclosure provides sustained release drug delivery devices in the form of a combined preparation, for example, as an admixture of two or more anti-connexin agents, for example one or more anti-connexin polynucleotides and one or more anti-connexin peptides or peptidomimetics. Also within the invention, as noted, are devices with mixtures of compounds that include one or more anti-cadherin and/or anti-catenin compounds.

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially, whether in pharmaceutical or scaffold form or dressing/matrix form or all of the above. The parts of the kit can then, for example, be administered simultaneously or chronologically staggered, that is at different time points and with equal or different time intervals for any part of the kit of parts.

In one aspect, a combined preparation is administered, wherein two or more separate compositions are administered to a subject, wherein the first composition comprises a therapeutically effective amount of an anti-connexin 26 polynucleotide and the second composition comprises a therapeutically effective amount of an anti-connexin 43 agent, e.g., an anti-connexin 43 polynucleotide, peptide, or peptidomimetic. In one aspect a third composition is administered comprising one or more anti-connexin polynucleotides, peptides, or peptidomimetics. The third composition may also comprise one or more gap junction closing compounds, hemichannel closing compounds, or connexin carboxy-terminal polypeptides, or one or more anti-cadherin and/or anti-catenin compounds useful for wound healing.

In one aspect, preferred anti-connexin agents, including anti-connexin oligonucleotides and anti-connexin peptides and peptidomimetics, are directed against connexin 26 and/or connexin 43.

In some aspects, the device includes sustained release drug delivery devices for administering a therapeutically effective amount of a first anti-connexin agent agents (e.g., an anti-connexin 26 agent and/or an anti-connexin 43 agent, for example an antisense polynucleotide, peptide, peptidomimetic, or small molecule hemichannel blocker, for example tonabersat or other hemichannel blocker), for example, a first anti-connexin 26 agent, preferably an anti-Cx26 polynucleotide, alone or in conjunction with a second anti-connexin agent, for example, one or more anti-connexin polynucleotides, preferably anti-connexin 43 antisense polynucleotide, and optionally one or more anti-connexin peptides or peptidomimetics, formulated in a delayed release preparation, a slow release preparation, an extended release preparation, a controlled release preparation, and/or in a repeat action preparation to a subject with a wound, including wounds characterized in whole or in part by delayed or incomplete wound healing. In some aspects the first or second anti-connexin agent may be, for example, any one of an anti-connexin 43, anti-connexin 37, anti-connexin 30, anti-connexin 31.1 or anti-connexin 32 agent.

This disclosure is also directed to a method of topical administration of a sustained release drug delivery device comprising a wound coating device, e.g. a scaffold, comprising, for example, an effective amount of one or more anti-connexin agents (e.g., an anti-connexin 26 agent and/or an anti-connexin 43 agent, for example an antisense polynucleotide, peptide, peptidomimetic, or small molecule for example tonabersat), in which an improvement comprises including a sustained release formulation of said one or more anti-connexin agents, which releases one or more anti-connexin agents into a medium comprising water. In one aspect, a method is provided for a topical administration of a sustained release drug delivery device comprising a pharmaceutical composition further comprising an effective amount of one or more anti-connexin agents (e.g., an anti-connexin 26 agent and/or an anti-connexin 43 agent, for example an antisense polynucleotide, peptide, peptidomimetic, or small molecule for example tonabersat), in which an improvement comprises including a sustained release formulation of said one or more anti-connexin agents, which releases one or more anti-connexin agents in or on the skin.

In some aspects, this disclosure also relates to methods of using sustained release drug delivery devices to treat subjects suffering from or at risk for various diseases, disorders, and conditions associated with a wound, including acute and wounds that do not heal at expected rates, including delayed healing and chronic wounds. In some aspects, the wound can be in the skin. In some aspects, the wound can be a partial thickness wound.

In some aspects, this disclosure includes methods for treating a subject having or suspected of having or predisposed to, or at risk for, any diseases, disorders and/or conditions characterized in whole or in part by a wound or a tissue in need of repair using the sustained release drug delivery devices described herein.

In one aspect, this disclosure provides method of treatment comprising administering to a subject a sustained release drug delivery device of the invention for use in the treatment of a wound, including for example, acute, as well as wounds that do not heal at expected rates, including delayed healing and chronic wounds, optionally a diabetic ulcer, a pressure ulcer, a vasculitic ulcer, or an arterial ulcer.

In one aspect, this disclosure provides a method of treatment comprising administering to a subject in need thereof a sustained release drug delivery device comprising therapeutically effective amounts of a first anti-connexin agent (e.g., an anti-connexin 26 agent and/or an anti-connexin 43 agent, for example an antisense polynucleotide, peptide, peptidomimetic, or small molecule for example tonabersat) and a second anti-connexin agent, wherein said first agent is an anti-connexin 26 polynucleotide agent and said second agent is an anti-connexin peptide or peptidomimetic different from the first anti-connexin 26 agent. In some aspects, the second anti-connexin agent can be an anti-connexin 43 agent, preferably an anti-connexin 43 antisense polynucleotide. In one aspect, the invention provides a method of treatment comprising administering to a subject in need thereof a sustained release drug delivery device comprising therapeutically effective amounts of a first anti-connexin 26 agent and a second anti-connexin agent, wherein said first agent is an anti-connexin 26 polynucleotide agent and said second agent is an anti-connexin 43 agent. In some aspects, the second anti-connexin 43 agent can be an anti-connexin 43 antisense polynucleotide, peptide, or small molecule (e.g., tonabersat). In some aspects the first or second anti-connexin agent may be, for example, any one of an anti-connexin 43, anti-connexin 37, anti-connexin 30, anti-connexin 31.1 or anti-connexin 32 agent.

In one aspect, this disclosure provides a method of treatment comprising administering to a subject in need thereof a sustained release drug delivery device comprising a therapeutically effective amount of an anti-connexin 26 polynucleotide, preferably with an effective amount of an anti-connexin 43 polynucleotide. In one aspect, this disclosure provides a method of treatment comprising administering to a subject in need thereof a sustained release drug delivery device comprising a combination of a therapeutically effective amount of an anti-connexin 26 polynucleotide and a therapeutically effective amount of an anti-connexin 43 polynucleotide.

In one aspect, this disclosure provides a method of treatment comprising administering to a subject in need thereof a sustained release drug delivery device comprising a first composition comprising a therapeutically effective amount of an anti-connexin 26 polynucleotide and a second composition comprising a therapeutically effective amount of an anti-connexin 43 peptide or peptidomimetic. In one aspect the first composition is administered first. In one aspect, the second composition is administered first. In one aspect, the method further comprises administration of a third composition, wherein the third wound healing composition comprises an anti-connexin polynucleotide, peptide or peptidomimetic. In one aspect the third composition is administered first. In some aspects, the treated wound exhibits a reduction of epidermal wound edge, reduction in granulation tissue area, and/or a reduction in the concentration of Myofibroblasts in the granulation tissue area.

In one aspect, this disclosure provides a method for treating acute wounds, comprising administering to a subject in need thereof a sustained release drug delivery device comprising a therapeutically effective amount of a pharmaceutical composition comprising a first anti-connexin agent (e.g., an anti-connexin 26 agent and/or an anti-connexin 43 agent, for example an antisense polynucleotide, peptide, peptidomimetic, or small molecule for example tonabersat) and a second anti-connexin agent as described herein, for example, one or more anti-connexin polynucleotides and one or more anti-connexin peptides or peptidomimetics. In one aspect, said method comprises administration of two pharmaceutical compositions, the first composition comprising one or more anti-connexin polynucleotides and the second pharmaceutical composition comprising one or more anti-connexin peptides or peptidomimetics. In one aspect the first composition is administered first. In one aspect, the second composition is administered first. In one aspect, the method further comprises administration of a third composition, wherein the third wound healing composition comprises an anti-connexin polynucleotide, peptide or peptidomimetic. In one aspect the third composition is administered first. In one aspect the third composition is administered first. In one aspect the pharmaceutical compositions are administered topically. In some aspects the first or second anti-connexin agent may be, for example, an anti-connexin 43, anti-connexin 37, anti-connexin 30, anti-connexin 31.1 or anti-connexin 32 agent.

In one aspect, this disclosure provides a method for treating chronic wounds, or delayed or slow healing wounds comprising administering to a subject in need thereof a sustained release drug delivery device comprising a therapeutically effective amount of an anti-connexin agent (e.g., an anti-connexin 26 agent and/or an anti-connexin 43 agent, for example an antisense polynucleotide, peptide, peptidomimetic, or small molecule for example tonabersat) alone or in combination with a second anti-connexin agent as described herein, for example, one or more anti-connexin polynucleotides and one or more anti-connexin peptides or peptidomimetics. In one aspect, said method comprises administration of one or two pharmaceutical compositions, the first composition comprising one or more anti-connexin 26 polynucleotide species and the second pharmaceutical composition comprising one or more anti-connexin agents that differ from anti-connexin 26 polynucleotide species in the first composition.

In some aspects the chronic wound is a diabetic ulcer, a diabetic foot ulcer, a venous ulcer, a venous stasis ulcer, a pressure ulcer, a decubitus ulcer, a vasculitic ulcer, an arterial ulcer, an infectious ulcer, a burn ulcer, a trauma-induced ulcer, or an ulceration associated with pyoderma gangrenosum. In one aspect the subject is diabetic. In one aspect the subject has a cardiovascular disease or condition. In one aspect, the chronic wound is a persistent epithelial defect.

In some aspects, this disclosure provides a method of reducing connexin 26 (Cx26) expression and connexin 43 (Cx43) expression in a cell, comprising delivering to a cell that expresses Cx26 and Cx43 from a sustained release drug delivery device comprising combination of an anti-Cx26 polynucleotide and an anti-Cx43 polynucleotide according to claim 4, thereby reducing Cx26 expression and Cx43 expression in the cell.

In one aspect of combination treatments, the first composition anti-connexin agent is administered first. In one aspect, the second composition is administered first. In some aspects, the method further comprises administration of a third composition, wherein the third wound healing composition comprises an anti-connexin agent, for example, an anti-connexin polynucleotide, peptide or peptidomimetic. In one aspect the methods of the present invention may be used to treat persistent epithelial defects. Application of the sustained release drug delivery devices of the present invention may improve healing of the epithelium and basement membrane complex. In one aspect the third composition is administered first. In one aspect the third composition is administered first. In one aspect the pharmaceutical compositions are administered topically by dissolution or degradation of the sustained release drug delivery device.

In one aspect, this disclosure provides a method for reducing scar formation in a subject in need thereof, comprising administering to said subject sustained release drug delivery device comprising a therapeutically effective amount of a pharmaceutical composition further comprising an anti-connexin 26 agent, e.g., an anti-Cx26 polynucleotide, alone or in combination with a second anti-connexin agent, for example, one or more anti-connexin polynucleotides, preferably an anti-Cx43 polynucleotide, and/or one or more anti-connexin peptides or peptidomimetics that differ from the anti-connexin 26 agent species. In one aspect, said method comprises administration of two pharmaceutical compositions, the first composition comprising one or more anti-connexin 26 polynucleotides and the second pharmaceutical composition comprising one or more anti-connexin polynucleotides, peptides, or peptidomimetics that target or mimic a connexin other than Cx26. In one aspect the first composition is administered first. In one aspect, the second composition is administered first. In one aspect, the method further comprises administration of a third composition, wherein the third wound healing composition comprises an anti-connexin agent, for example, an anti-connexin polynucleotide, peptide or peptidomimetic.

Preferred methods include the sole, sequential, or substantially simultaneous administration one or more anti-connexin agent species from the dissolution or degradation of the dosage form scaffold, for example, one or more anti-connexin polynucleotides alone or in combination with one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone.

In one aspect, this disclosure includes transdermal patches, dressings, pads, wraps, matrices and bandages capable of being adhered or otherwise associated with the skin of a subject, said articles being capable of contacting a wound with a dosage form described herein for delivering a therapeutically effective amount of an anti-connexin 26 agent alone or in combination with an anti-connexin 43 agent.

In one aspect, this disclosure includes articles of manufacture comprising a sustained release drug delivery device comprising, for example, a therapeutically effective amount of an anti-connexin 26 agent, preferably an anti-Cx26 polynucleotide alone or together with and a different, second anti-connexin agent, preferably an anti-Cx43 polynucleotide, and others as noted herein, in addition to instructions for use of such anti-connexin agent(s), including use for the treatment of a subject.

In one aspect, this disclosure includes articles of manufacture that comprise packaging material containing one or more sustained release drug delivery devices containing an anti-connexin 26 agent, preferably an anti-Cx26 polynucleotide, alone or together with a second anti-connexin agent, preferably an anti-Cx43 polynucleotide, wherein the packaging material has a label that indicates that the sustained release drug delivery device(s) can be used for a subject having or suspected of having or predisposed to any of the diseases, disorders, and/or conditions described or referenced herein, including diseases, disorders, and/or conditions characterized in whole or in part by acute, impaired, delayed, or chronic wound healing. Such sustained release drug delivery devices include, for example, scaffold, including collagen scaffold, forms and formulations.

In one aspect, this disclosure includes methods for the use of dosage forms comprising therapeutically effective amounts of compositions described herein in the manufacture of sustained release drug delivery devices. Such sustained release drug delivery devices include, for example, scaffold, including collagen scaffold, forms and formulations. Such devices include those for the treatment of a subject as disclosed herein. Such devices, when used to deliver combinations of anti-connexin agents, one of which is an anti-connexin 26 agent, preferably an anti-Cx26 polynucleotide, preferably include the reduced amounts of the anti-connexin 26 agent and the second anti-connexin agent.

In one aspect, this disclosure includes methods of preparing a sustained release drug delivery device for treating a wound, comprising bringing together and an amount of an anti-connexin 26 agent, preferably an anti-Cx26 polynucleotide, alone or in combination with a second anti-connexin agent, preferably an anti-Cx43 polynucleotide. In such methods, the devices comprise an effective amount of an anti-connexin 26 polynucleotide and a second composition that comprises an effective amount of a different anti-connexin species, for example. Some aspects include preparing devices that include one or more anti-connexin 26 agents, a gap junction closing compound useful for wound healing, a hemichannel closing compound useful for wound healing, a connexin carboxy-terminal polypeptide useful for wound healing, and/or an anti-Cx43 polynucleotide.

In one aspect, this disclosure includes methods for the use of a therapeutically effective amount of an anti-connexin 26 agent, preferably an anti-Cx26 polynucleotide, alone or in combination with a second anti-connexin agent, preferably an anti-Cx43 polynucleotide, in the manufacture of sustained release drug delivery devices. Such sustained release drug delivery devices include, for example, scaffold, including collagen scaffold, forms and formulations. Such sustained release drug delivery devices include those for the treatment of a subject as disclosed herein. Such sustained release drug delivery devices preferably include the reduced amounts of the one or more anti-connexin agents, or reduced amounts of a gap junction closing compound useful for wound healing, a hemichannel closing compound useful for wound healing, and/or a connexin carboxy-terminal polypeptide useful for wound healing, for example.

In one aspect, this disclosure includes methods for administering a therapeutically effective amount of a second anti-connexin agent, preferably an anti-connexin 43 agent, wherein administration of anti-Cx26 polynucleotide and second anti-connexin agent is substantially simultaneously or staggered in time, optionally within at least about one-half hour of each other, within about one hour of each other, within about one day of each other, or within about one week of each other. In one aspect, the administration can be substantially simultaneous by dissolving the two anti-connexin agents in the same coatings of the coated scaffold. In one aspect, the administration can be staggered in time by dissolving a first anti-connexin agent in a first coating, and a second anti-connexin agent in a second coating. The dissolution or degradation of the outer (first) coating (first or second) can release the first anti-connexin agent first. Dissolution or degradation of the inner (second) coating can release the second anti-connexin agent second. In some aspects, the anti-Cx26 polynucleotide is administered before the second anti-connexin agent, preferably an anti-Cx43 agent, is administered. In some aspects, the second anti-connexin agent, preferably an anti-Cx43 agent, is administered before the anti-Cx26 polynucleotide is administered.

In some aspects, this disclosure provides for the use of a first anti-connexin agent and a second anti-connexin agent as described herein, for example, an anti-connexin polynucleotide and optionally an anti-connexin peptide or peptidomimetic, in the manufacture of a pharmaceutical product for the promotion of wound healing in a patient in need thereof.

In some aspects, the invention provides: (i) a package and/or article of manufacture comprising a sustained release drug delivery device comprising an anti-connexin 26 agent together with another anti-connexin agent, preferably an anti-connexin 43 agent, for the promotion (e.g., decrease in healing time, better wound outcome) of wound healing or tissue repair, (ii) a package comprising one or more devices with anti-connexin polynucleotides, at least on which is an anti-connexin 26 agent, preferably an anti-Cx26 polynucleotide, and preferably in combination with one or more other anti-connexin agents, preferably anti-connexin 43 agents, e.g., anti-connexin 43 polynucleotides, anti-connexin 43 peptides, or anti-connexin 43 peptidomimetics, or anti-connexin 43 small molecule hemichannel blockers (e.g., tonabersat) for the promotion of wound healing or tissue repair; and (iii) a package comprising one or more anti-connexin polynucleotide devices, at least one of which is an anti-connexin 26 agent, preferably an anti-Cx26 polynucleotide, together with one or more different anti-connexin polynucleotides or agents, preferably anti-Cx43 polynucleotides or peptides, or anti-Cx43 peptides peptidomimetics, or anti-Cx43 small molecule hemichannel blockers (e.g., tonabersat) together with instructions for use in the promotion or improvement of wound healing or tissue repair.

In a one aspect the dosage form(s) of the invention is(are) provided in combination with a topically administered device, for example a wound scaffold, wound dressing or wound healing promoting matrix. Suitably a scaffold, dressing or matrix is provided a coating or a solid or liquid substrate into or onto which the anti-connexin agent(s) (e.g., an anti-connexin 26 agent, preferably an anti-Cx26 polynucleotide, and/or an anti-connexin 43 agent, preferably an anti-Cx43 polynucleotide) is(are) dispersed.

When used in combination, the first anti-connexin agent and second anti-connexin agents may be administered in the same composition or by separate compositions. Preferably, when combinations of different anti-connexin agents are administered, a first anti-connexin agent to be delivered is an anti-connexin agent that can block or reduce hemichannel opening, and the anti-connexin 26 and/or 43 polynucleotide blocks or reduce connexin expression or the formation of hemichannels or gap junctions, e.g., by downregulation of connexin protein expression. In some aspects, order of administering the anti-Cx26 agent and the anti-Cx43 agent can be controlled by adding the respective agent to a different biodegradable polymer layer on the scaffold or other sustained release drug delivery device described herein.

These and other aspects of the present inventions, which are not limited to or by the information in this Brief Summary, are provided below.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 is an illustration of ASN based on DNAzyme walk. A schematic diagram of DNAzyme is also shown.

FIG. 2 shows Cx26, Cx30, and Cx43 mRNA expression in human mammary epithelial cells (HMEpCs) and in positive controls.

FIG. 3A shows Cx43 expression at the protein level in HMEpC cells. FIG. 3B shows Cx26 expression at the protein level in HMEpC cells. And FIG. 3C shows Cx30 expression at the protein level in HMEpC cells.

FIG. 4 shows Cx26, Cx30, and Cx43 mRNA expression in HaCaT cells and in positive controls.

FIG. 5 shows Cx26, Cx30, and Cx43 mRNA expression in HepG2 cells and in positive controls.

FIGS. 6A, 6B and 6C show panels of FACS results for human umbilical vein endothelial cells (HUVEC) and HMEpC cells at 4 hr. post-transfection with a FAM-tagged LP2-PTO antisense polynucleotide. FIG. 6A is a micrograph showing results for HUVEC cells. FIG. 6B is a micrograph showing results for HMEpC cells. FIG. 6C is a table summarizing the results.

FIG. 7 is a bar graph showing the mean Cx26 RNA knockdown at 4 hr. post-transfection in HMEpC.

FIG. 8 shows a drawing of a rat indicating the placement of dorsal wounds excisions.

FIG. 9 is a photograph showing a representative example of an epithelial re growth measurement. In the photo, the dashed line indicates the length of epithelial re-growth.

FIG. 10 has 9 photographs showing the expression and localisation of connexin 26 (Cx26: green) at the wound edge of 6 mm wounds 6 hours post-wounding.

FIG. 11 shows a series of data plots from experiments examining wound re-epithelialization after administration of an anti-Cx26 antisense polynucleotide.

FIG. 12 is an illustration showing the process of preparing a scaffold coated with one or more anti-Cx antisense polynucleotides.

FIG. 13 is a graph showing the release profile of an anti-Cx43 ASN polynucleotide from a PCL scaffold.

FIG. 14 illustrates a controlled release delivery device having a scaffold coated with two different PLGA coatings, each of which contains an anti-Cx43 ASN polynucleotide.

FIG. 15 is a graph showing the elution profiles of an anti-Cx43 ASN polynucleotide from a controlled release delivery device having a scaffold coated with PCL, PLDA, or a mixture of PCL and PLGA.

FIG. 16A illustrates an exemplary process of preparing scaffolds coated with one or more polymers with anti-Cx antisense polynucleotides. FIG. 16B is a graph showing the reproducibility of the cumulative elution profiles of an anti-Cx43 ASN polynucleotide from a controlled release delivery device having a scaffold coated with PCL, PLDA, or a mixture of PCL and PLGA with 1 coating layer (compare with FIG. 15). FIG. 16C is a graph showing the cumulative elution profiles of an anti-Cx43 ASN polynucleotide from a controlled release delivery device having a scaffold coated with PCL, PLDA, or a mixture of PCL and PLGA with 4 coating layers. FIG. 16D is a graph showing the freshly released elution profiles of an anti-Cx43 ASN polynucleotide from a controlled release delivery device having a scaffold coated with PCL, PLDA, or a mixture of PCL and PLGA with 4 coating layers.

FIGS. 17A, 17B, 17C and 17D shows the FRET experiment and results. FIG. 17A is an illustration showing the experimental setup on the scaffold being interrogated. Simplified diagram of asODN coated scaffold illustrating the stochastic dispersion of asODN clusters across the scaffold as well as the site at which images were captured in (D). FIG. 17B is a graph showing the FRET efficiency of the FRET pair as a function of serum exposure time. Scaffolds were submerged in FBS for up to 7 days, after which they were sectioned and acceptor bleached using a confocal 633 nm wavelength laser. FRET efficiency was calculated as a percentage increase in donor fluorescence intensity after acceptor bleaching relative to before acceptor bleaching. A total of 6 regions were quantified per time point, graph data=means+SEM error bars. FIG. 17C is a graph showing the lambda scan of treated versus untreated scaffolds. xyλ scans were performed on a 300 μM asODN solution either treated or untreated with FBS for 8 h to test the potency of the FBS as a nuclease effective against asODN. The mean fluorescence intensity values detected at 19 steps between 550 nm and 730 nm following excitation with a 533 nm laser were plotted. Six regions were assessed to generate averages. FIG. 17D is the images of the scaffold impregnated with the FRET pair pre- and post-bleaching. A typical confocal image of a scaffold section following immersion in FBS used to generate the FRET efficiencies plotted in (B). Images show labelled asODN clusters both before and after Cy5 bleaching. Scale bar=20 μm.

FIGS. 18A and 18B show the experimental setup and images of the in vivo wound healing study. FIG. 18A is an illustration showing the experimental setup of the scaffold types and placement on the rat subject. FIG. 18B shows visual images of the treatment subjects' wounds at specific time points (in days).

FIGS. 19A and 19B show the histology of the wound edge at Day 1 of wound healing. FIG. 19A shows the experimental setup. FIG. 19B shows the re-epithelialization distance both visually (in stained samples) and graphically.

FIGS. 20A and 10B show the histology of the wound edge at Day 3 of wound healing. FIG. 20A shows the experimental setup. FIG. 20B shows the re-epithelialization distance both visually (in stained samples) and graphically.

FIGS. 21A, 21B, 21C and 21D show the histology of the wound edge at Day 5 of wound healing. FIG. 21A shows the experimental setup. FIG. 21B shows the re-epithelialization distance graphically. FIG. 21C is a graph showing the re-epithelialization distance of the wound edge of the treatment groups at Days 1, 3, and 5 of wound healing. FIG. 21D shows the images (of stained samples) of the wound edge for the treatment groups.

FIG. 22 shows the Cx26-specific stain images and area volume graph of the wound edge at Day 1 of wound healing.

FIG. 23 shows the Cx43-specific stain images and area volume graph of the wound edge at Day 1 of wound healing.

FIG. 24 shows the Cx26-specific stain images and area volume graph of the wound edge at Day 5 of wound healing.

FIG. 25 shows the Cx43-specific stain images and area volume graph of the wound edge at Day 5 of wound healing

FIG. 26 shows the histology of granulation tissue area at Day 10 of wound healing. Shown are the experimental setup of the image viewed, the images of stained samples of the treatment groups, and a graph showing the measured granulation tissue areas.

FIG. 27 shows the histology of granulation tissue area at Day 15 of wound healing. Shown are the experimental setup of the image viewed, the images of stained samples of the treatment groups, and a graph showing the measured granulation tissue areas.

FIG. 28 shows the smooth muscle actin staining of myofibroblasts in granulation tissue area at Day 10 of wound healing.

FIGS. 29A, 29B, 29C, 29D and 29E show Cx43 asODN elution from differing combinations of scaffold coatings. FIG. 29A shows electrospun collagen scaffolds were dipped in an emulsion of 100 μM (micromolar) Cx43 asODN and either 10% or 15% PLGA and immediately subject to freezing and lyophilisation. FIG. 29B shows cumulative Cx43 asODN elution from scaffolds was quantified in water at 37° C. over 4 days. FIG. 29C shows schematic illustrating the procedure used to create scaffolds with multiple coating layers. FIG. 29D shows elution profile from scaffolds subject to one round of processing. FIG. 29E shows elution profile from scaffolds subject to four rounds of processing. The average of three samples was recorded for all elution measurements and error bars represent SEM.

FIGS. 30A and 30B show macroscopic wound evaluation following coated scaffold placement. FIG. 30A shows full thickness rat wounds were treated with one of a number of different collagen scaffolds or left untreated. FIG. 30B shows animals were culled at either 1, 3 or 5 days after wounding and the wounds macroscopically imaged. Scale bar=1 mm.

FIGS. 31A, 31B, 31C and 31D shows Day 1 full thickness wound healing following application of the various scaffolds. FIG. 31A shows panel showing representative H&E and immunofluorescent staining (Cx43 or Cx26 in green; Hoechst in blue) images from wounds subject to the indicated treatments. Re-epithelialisation is marked by yellow dotted lines on the H&E stains. Epithelium is marked by white dotted lines on the immunofluorescent stains. FIG. 31B shows wound re-epithelialisation distances. FIG. 31C shows percentage change in epidermal wound edge Cx43 normalized to regions of distal epidermis. FIG. 31D shows percentage change in epidermal wound edge Cx26 normalized to regions of distal epidermis. Plotted as means+SEM error bars. Eight rat wounds per condition were assessed. Dunnett's test conducted against the untreated control (*p<0.05, **p<0.01, ***p<0.001). Scale bar=100 μm (microns).

FIGS. 32A, 32B, 32C and 32D show Day 3 full thickness wound healing following application of the various scaffolds. FIG. 32A shows panel showing representative H&E and immunofluorescent staining (Cx43 or Cx26 in green; Hoechst in blue) images from wounds subject to the indicated treatments. Re-epithelialisation is marked by yellow dotted lines on the H&E stains. Epithelium is marked by white dotted lines on the immunofluorescent stains. FIG. 32B shows wound re-epithelialisation distances. FIG. 32C shows percentage change in epidermal wound edge Cx43 normalized to regions of distal epidermis. FIG. 32D shows percentage change in epidermal wound edge Cx26 normalized to regions of distal epidermis. Plotted as means+SEM error bars. Eight rat wounds per condition were assessed. Dunnett's test conducted against the untreated control (*p<0.05, **p<0.01, ***p<0.001). Scale bar=100 μm (microns).

FIGS. 33A, 33B, 33C and 33D show Day 5 full thickness wound healing following application of the various scaffolds. FIG. 33A shows panel showing representative H&E and immunofluorescent staining (Cx43 or Cx26 in green; Hoechst in blue) images from wounds subject to the indicated treatments. Re-epithelialisation is marked by yellow dotted lines on the H&E stains. Epithelium is marked by white dotted lines on the immunofluorescent stains. FIG. 33B shows pound re-epithelialisation distances. FIG. 33C shows percentage change in epidermal wound edge Cx43 normalized to regions of distal epidermis. FIG. 33D shows percentage change in epidermal wound edge Cx26 normalized to regions of distal epidermis. Plotted as means+SEM error bars. Eight rat wounds per condition were assessed. Dunnett's test conducted against the untreated control (*p<0.05, **p<0.01, ***p<0.001). Scale bar=100 μm (micrometers).

FIGS. 34A, 34B and 34C shows the effect of scaffold application on epidermal thickening and dermal infiltration of polymorphonuclear cells. FIG. 34A shows full thickness wounds treated with different types of scaffolds were measured for epithelial thickness within the end 150 μm of the nascent tip of epidermis. Measurements were recorded across n=8 samples per timepoint. FIG. 34B shows full-thickness wounds that received a scaffold treatment were assessed for polymorphonuclear cell invasion into the lower dermis 700 μm away from the wound edge as indicated in the topleft of diagram. E=epidermis, D=dermis, PC=panniculus carnosus, WE=wound edge. High-power typical images of the dermal region are also shown for each scaffold treatment. The typical images shown are of the unwounded dermis distal to day 3 wounds. Scale bar=50 μm. FIG. 34C shows quantification of polymorphonuclear cells for each treatment. Plotted as average values of n=5 samples per timepoint. Error bars represent SEM. Dunnett's test conducted against the untreated control (*p<0.05, **p<0.01, ***p<0.001).

FIGS. 35A, 35B and 35C shows the long-term full thickness wound healing following application of the various scaffolds. FIG. 35A shows panel showing representative H&E images from day 10 wounds subject to the indicated treatments. Granulation tissue area is marked by white dotted lines. FIG. 35B shows quantification of granulation tissue area on day 10 and 15. FIG. 35C shows quantification of granulation tissue area on day 15. Plotted as average values of n=8 samples per timepoint. Error bars represent SEM. Dunnett's test conducted against the untreated control (*p<0.05, **p<0.01, ***p<0.001). Scale bar=100 μm.

### DETAILED DESCRIPTION

As used herein, a “disorder” is any disorder, disease, or condition that would benefit from an agent described herein, including those that promotes wound healing and/or reduces swelling, inflammation, and/or scar formation. For example, included are wounds resulting from surgery or trauma, and wound-associated abnormalities in connection with neuropathic, ischemic, microvascular pathology, pressure over bony area (tailbone (sacral), hip (trochanteric), buttocks (ischial), or heel of the foot), reperfusion injury, and valve reflux etiology and conditions. Also included are chronic wounds and other impaired healing wounds including dehiscent wounds.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats.

As used herein, “preventing” means preventing in whole or in part, or ameliorating or controlling.

As used herein, a “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result in various applications will involve the promotion and/or improvement of wound healing, including rates of wound healing and closure of wounds, in whole or in part. Other benefits include decreases in swelling, inflammation and/or scar formation, in whole or in part.

As used herein, the terms “treating” and “treatment” refer to both therapeutic treatment and prophylactic or preventative measures.

As used herein, “anti-connexin agents” are compounds that affect or modulate the activity, expression or formation of a connexin, a connexin hemichannel (connexon), or a gap junction. Anti-connexin agents include, without limitation, antisense compounds (e.g., antisense polynucleotides), RNAi and siRNA compounds, unlocked and locked RNA compounds, antibodies and binding fragments thereof, and peptides and polypeptides, which include “peptidomimetics,” and peptide analogs. Anti-connexin agents also include gap junction closing compounds useful for wound healing (e.g., connexin phosphorylation compounds, and C-terminal ZO-1 binding peptides), hemichannel closing compounds useful for wound healing (e.g., connexin phosphorylation compounds, and C-terminal ZO-1 binding peptides), connexin peptides comprising extracellular domains and/or comprising connexin carboxy-terminal peptides useful for wound healing, and small molecule hemichannel blocking or closing compounds. Preferred anti-connexin agents are anti-connexin 26 agents, anti-connexin 26 gap junction agents, anti-connexin 26 hemichannel agents, anti-connexin 43 agents, anti-connexin 43 gap junction agents, and anti-connexin 43 hemichannel agents. In some aspects the first or second anti-connexin agent may be, for example, an anti-connexin 43, anti-connexin 37, anti-connexin 30, anti-connexin 31.1 or anti-connexin 32 agent. Exemplary anti-connexin agents are discussed in further detail herein.

As used herein, the term “accessible site” refers to a plurality of contiguous nucleotide residues in a nucleic acid molecule that can be bound by a protein or hybridized by another nucleic acid molecule having a partially or completely complementary nucleotide sequence over the length of the accessible site under physiological conditions. Here, “physiological conditions” means those conditions that exist within a living cell under which the particular chemical reaction (e.g., nucleic acid hybridization) is to occur, or such other conditions (e.g., laboratory conditions) designed to approximate those within a living cell to such an extent that experimental results (e.g., the hybridization of a probe nucleic acid to its target sequence) achieved under such other conditions can also be achieved in a living cell.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks or months after the other provided that both the one or more anti-connexin polynucleotides and one or more anti-connexin peptides or peptidomimetics, for example, are concurrently present in effective amounts. The time delay between administration or administrations of the components will vary depending on the exact nature of the components, the interaction there between, and their respective half-lives. “Substantially simultaneously” means less than about one minute. Thus, when two agents are administered “substantially simultaneously”, they are administered within less than about one minute of each other.

The terms “peptidomimetic” and “mimetic” include naturally occurring and synthetic chemical compounds that may have substantially the same structural and functional characteristics of protein regions that they mimic. In the case of connexins, these may mimic, for example, the extracellular loops of opposing connexins involved in connexon-connexon docking and cell-cell channel formation, and/or the extracellular loops of hemichannel connexins.

As used herein, the term “peptide analogs” refer to the compounds with properties analogous to those of the template peptide and can be non-peptide drugs. “Peptidomimetics” (also known as peptide mimetics) which include peptide-based compounds, also include such non-peptide based compounds such as peptide analogs. Peptidomimetics that are structurally similar to therapeutically useful peptides can be used to produce an equivalent or enhanced therapeutic or prophylactic effect. Generally, peptidomimetics are structural or functional mimics (e.g., identical or similar) to a paradigm polypeptide (i.e., a polypeptide that has a biological or pharmacological function or activity), but can also have one or more peptide linkages optionally replaced by a linkage selected from the group consisting of, for example, —CH2NH—, —CH2S—, —CH2-CH2-, —CH═CH— (cis and trans), —COCH2-, —CH(OH)CH2-, and —CH2SO—. The mimetic can be either entirely composed of natural amino acids, synthetic chemical compounds, non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. In the case of connexins, these can mimic, for example, the extracellular loops of opposing connexins involved in connexon-connexon docking and cell-cell channel formation. For example, a mimetic composition can be useful as a gap junction-modulating agent if it is capable of down-regulating biological actions or activities of connexons, such as, for example, preventing the docking of connexons to form gap-junction-mediated cell-cell communications, or preventing the opening of connexons to expose the cell cytoplasm to the extracellular milieu. Peptidomimetics encompass those described herein, as well as those as may be known in the art, whether now known or later developed.

In general, the terms “modulator” and “modulation” of connexin activity, as used herein in its various forms, refers to inhibition in whole or in part of the action or activity of a connexin or a connexin hemichannel or connexin gap junction and may function as anti-connexin agents, including as gap junction modulation agents.

As used herein, the term “protein” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). In some embodiments, the peptides described above, and herein, are synthetic peptides. In some embodiments, the synthetic peptides are not found in nature. Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

The term “wound dressing” refers to a dressing for topical application to a wound and excludes compositions suitable for systemic administration. For example, the one or more anti-connexin agents, including gap junction modulation agents, and/or other agents, may be dispersed in or on a solid sheet of wound contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example. In certain embodiments the one or more anti-connexin agents, including gap junction modulation agents and/or other agents are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “matrix” includes for example, matrices such as collagen, acellular matrices, and crosslinked biological scaffold molecules. It can also include tissue-based matrices (including pig-based wound healing matrices), cultured epidermal autografts, cultured epidermal allografts, tissue-engineered skin, collagen, and glycosaminoglycan dermal matrices inoculated with autologous fibroblasts and keratinocytes, Alloderm (a nonliving allogeneic acellular dermal matrix with intact basement membrane complex), living skin equivalents (e.g., Dermagraft (living allogeneic dermal fibroblasts grown on degradable scaffold), TransCyte (an extracellular matrix generated by allogeneic human dermal fibroblasts), Apligraf (a living allogeneic bilayered construct containing keratinocytes, fibroblasts, and bovine type I collagen), and OrCel (allogeneic fibroblasts and keratinocytes seeded in opposite sides of bilayered matrix of bovine collagen), animal derived dressings (e.g., Oasis's porcine small intestinal submucosa acellular collagen matrix; and E-Z Derm's acellular xenogeneic collagen matrix), tissue-based bioengineered structural frameworks, biomanufactured bioprostheses, and other implanted or applied structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free, or antigen reduced acellular matrix (Wilson, et al., Trans Am Soc Artif Intern 1990; 36:340-343) or other biomatrix that have been engineered to reduce the antigenic response to the xenograft material. Other matrix materials useful in promotion of wound healing may include for example, processed bovine pericardium proteins comprising insoluble collagen and elastin (Courtman, et al., J Biomed Mater Res 1994; 28:655-666) and other acellular tissue which may be useful for providing a natural microenvironment for host cell migration to accelerate tissue regeneration (Malone, et al., J Vasc Surg 1984; 1:181-91). In certain embodiments, the matrix material is supplemented with one or more anti-connexin polynucleotides and/or the one or more anti-connexin peptides or peptidomimetics for site-specific release of such agents, as noted herein. Matrices may also comprise suitable antimicrobial agents, for example, silver and polyhexamethylene biguanide (PHMB), to maintain efficacy against infection. In some aspects, the silver antimicrobial agents can be silver nanoparticles, with a mean diameter of 5 to 600 nanometers. In some aspects, the silver nanoparticle antimicrobial agents can be surface functionalized to enhance dissolution or solution processing when preparing the matrix material.

As used herein, “wound promoting matrix” includes synthetic or naturally occurring matrices. They include, for example, collagen, acellular matrices, crosslinked biological scaffold molecules, tissue based bioengineered structural framework, biomanufactured bioprostheses, and other implanted structures such as for example, vascular grafts suitable for cell infiltration and proliferation useful in the promotion of wound healing. Additional suitable biomatrix materials include chemically modified collagenous tissue to reduce antigenicity and immunogenicity. Other suitable examples include collagen sheets for wound dressings, and antigen-free, or antigen-reduced acellular matrices (Wilson, et al. (1990) Trans Am Soc Artif Intern 36:340-343) or other biomatrix materials that have been prepared to reduce the antigenic response to the xenograft material. Other matrices useful in promotion of wound healing may include, for example, processed bovine pericardium proteins comprising insoluble collagen and elastin (Courtman, et al. (1994) J Biomed Mater Res 28:655-666) and other acellular tissue which may be useful for providing a natural microenvironment for host cell migration to accelerate tissue regeneration (Malone, et al. (1984) J Vasc Surg 1:181-91). The invention contemplates a synthetic or natural matrix comprising one or more anti-connexin agents and/or one or more other agents, such as anti-cadherin and/or anti-catenin agents.

### Wounds and Wound Classification

As used herein, the term “wound” includes an injury to any tissue, including, for example, acute, delayed, or difficult to heal wounds, and chronic wounds. Examples of wounds may include or exclude, for example, both open and closed wounds. Wounds include, for example, burns, incisions, excisions, lacerations, abrasions, puncture on penetrating wounds, surgical wounds, contusions, hematoma, crushing injuries, and ulcers. Also included are wounds that do not heal at expected rates. The term “wound” may also include for example, injuries to the skin and subcutaneous tissue initiated in different ways (e.g., pressure sores from extended bed rest and wounds induced by trauma) and with varying characteristics. Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

The term “partial thickness wound” refers to wounds that encompass Grades I-III; examples of partial thickness wounds include pressure sores, venous stasis ulcers, and diabetic ulcers. The present invention contemplates treating all wounds of a type that do not heal at expected rates, including, delayed-healing wounds, incompletely healing wounds, and chronic wounds.

By “wound that does not heal at an/the expected rate” is meant an injury to any tissue that does not heal in an expected or typical time frame, including delayed or difficult to heal wounds (including delayed or incompletely healing wounds), and chronic wounds. Examples of wounds that do not heal at the expected rate include ulcers such as diabetic ulcers, diabetic foot ulcers, vascultic ulcers, arterial ulcers, venous ulcers, venous stasis ulcers, burn ulcers, infectious ulcers, trauma-induced ulcers, pressure ulcers, decubitus ulcers, ulcerations associated with pyoderma gangrenosum, and mixed ulcers. Other wounds that do not heal at expected rates include dehiscent wounds.

As used herein, a delayed or difficult to heal wound may include, for example, a wound that is characterized at least in part by 1) a prolonged inflammatory phase, 2) a slow forming extracellular matrix, and/or 3) a decreased rate of epithelialization or closure.

The term “chronic wound” refers to a wound that has not healed. Wounds that do not heal within three months, for example, are considered chronic. Chronic wounds include, for example, pressure ulcers, decubitus ulcers, diabetic ulcers including diabetic foot and leg ulcers, slow or non-healing venous ulcers, venous stasis ulcers, arterial ulcers, vasculitic ulcers, burn ulcers, trauma-induced ulcers, infectious ulcers, mixed ulcers, and pyoderma gangrenosum. The chronic wound may be an arterial ulcer that comprises ulcerations resulting from complete or partial arterial blockage. The chronic wound may be a venous or venous stasis ulcer that comprises ulcerations resulting from a malfunction of the venous valve and the associated vascular disease. In certain embodiments a method of treating a chronic wound is provided where the chronic wound is characterized by one or more of the following AHCPR stages of pressure ulceration: stage 1, stage 2, stage 3, and/or stage 4.

As used herein, chronic wound may also include, for example, a wound that is characterized at least in part by 1) a chronic self-perpetuating state of wound inflammation, 2) a deficient and defective wound extracellular matrix, 3) poorly responding (senescent) wound cells (including fibroblasts), 4) limited extracellular matrix production, and/or 5) failure of re-epithelialization due in part to lack of the necessary extracellular matrix orchestration and lack of scaffold for migration. Chronic wounds may also be characterized by 1) prolonged inflammation and proteolytic activity leading to ulcerative lesions, including for example, diabetic, pressure (decubitous), venous, and arterial ulcers; 2) progressive deposition of matrix in the affected area, 3) longer repair times, 4) less wound contraction, 5) slower re-epithelialization, and 6) increased thickness of granulation tissue.

Exemplary chronic wounds may include “pressure ulcer.” Exemplary pressure ulcers may include all 4 stages of wound classifications based on AHCPR (Agency for Health Care Policy and Research, U.S. Department of Health and Human Services) guidelines, including for example, Stage 1. A stage I pressure ulcer is an observable pressure related alteration of intact skin whose indicators as compared to the adjacent or opposite area on the body may include changes in one or more of the following: skin temperature (warmth or coolness), tissue consistency (firm or boggy feel) and/or sensation (pain, itching). The ulcer appears as a defined area of persistent redness in lightly pigmented skin, whereas in darker skin tones, the ulcer may appear with persistent red, blue, or purple hues. Stage 1 ulceration may include nonblanchable erythema of intact skin and the heralding lesion of skin ulceration. In individuals with darker skin, discoloration of the skin, warmth, edema, induration, or hardness may also be indicators of stage 1 ulceration. Stage 2: stage 2 ulceration may be characterized by partial thickness skin loss involving epidermis, dermis, or both. The ulcer is superficial and presents clinically as an abrasion, blister, or shallow crater. Stage 3: stage 3 ulceration may be characterized by full thickness skin loss involving damage to or necrosis of subcutaneous tissue that may extend down to, but not through, underlying fascia. The ulcer presents clinically as a deep crater with or without undermining of adjacent tissue. Stage 4: stage 4 ulceration may be characterized by full thickness skin loss with extensive destruction, tissue necrosis, or damage to muscle, bone, or supporting structures (e.g., tendon, joint capsule). In certain embodiments a method of treating a chronic wound is provided where the chronic wound is characterized by one or more of the following AHCPR stages of pressure ulceration: stage 1, stage 2, stage 3, and/or stage 4.

Exemplary chronic wounds may include “decubitus ulcers.” Exemplary decubitus ulcers may arise as a result of prolonged and unrelieved pressure over a bony prominence that leads to ischemia. The wound tends to occur in patients who are unable to reposition themselves to off-load weight, such as paralyzed, unconscious, or severely debilitated persons. As defined by the U.S. Department of Health and Human Services, the major preventive measures include identification of high-risk patients; frequent assessment; and prophylactic measures such as scheduled repositioning, appropriate pressure-relief bedding, moisture barriers, and adequate nutritional status. Treatment options may include for example, pressure relief, surgical and enzymatic debridement, moist wound care, and control of the bacterial load. In certain embodiments a method of treating a chronic wound is provided wherein the chronic wound is characterized by decubitus ulcer or ulceration that results from prolonged, unrelieved pressure over a bony prominence that leads to ischemia. In certain embodiments a method of treating a chronic wound is provided wherein the chronic wound is characterized by decubitus ulcer or ulceration that results from prolonged, unrelieved pressure over a bony prominence that leads to ischemia.

Exemplary chronic wounds may include “arterial ulcers.” Chronic arterial ulcers are generally understood to be ulcerations that accompany arteriosclerotic and hypertensive cardiovascular disease. They are painful, sharply marginated, and often found on the lateral lower extremities and toes. Arterial ulcers may include those ulcers characterized by complete or partial arterial blockage that may lead to tissue necrosis and/or ulceration. Signs of arterial ulcer may include, for example, pulselessness of the extremity; painful ulceration; small, punctate ulcers that are usually well circumscribed; cool or cold skin; delayed capillary return time (briefly push on the end of the toe and release, normal color should return to the toe in about 3 seconds or less); atrophic appearing skin (for example, shiny, thin, dry); and loss of digital and pedal hair.

Exemplary chronic wounds may include “venous ulcers.” Exemplary venous ulcers may include the most common type of ulcer affecting the lower extremities and may be characterized by malfunction of the venous valve. The normal vein has valves that prevent the backflow of blood. When these valves become incompetent, the backflow of venous blood causes venous congestion. Hemoglobin from the red blood cells escapes and leaks into the extravascular space, causing the brownish discoloration commonly noted. It has been shown that the transcutaneous oxygen pressure of the skin surrounding a venous ulcer is decreased, suggesting that there are forces obstructing the normal vascularity of the area. Lymphatic drainage and flow also plays a role in these ulcers. The venous ulcer may appear near the medial malleolus and usually occurs in combination with an edematous and indurated lower extremity; it may be shallow, not too painful and may present with a weeping discharge from the affected site. In certain embodiments a method of treating a chronic wound is provided wherein the chronic wound is characterized by arterial ulcers or ulcerations due to complete or partial arterial blockage.

Exemplary chronic wounds may include “venous stasis ulcers.” Stasis ulcers are lesions associated with venous insufficiency are more commonly present over the medial malleolus, usually with pitting edema, varicosities, mottled pigmentation, erythema, and nonpalpable petechiae and purpura. The stasis dermatitis and ulcers are generally pruritic rather than painful. Exemplary venous stasis ulcers may be characterized by chronic passive venous congestion of the lower extremities results in local hypoxia. One possible mechanism of pathogenesis of these wounds includes the impediment of oxygen diffusion into the tissue across thick perivascular fibrin cuffs. Another mechanism is that macromolecules leaking into the perivascular tissue trap growth factors needed for the maintenance of skin integrity. Additionally, the flow of large white blood cells slows due to venous congestion, occluding capillaries, becoming activated, and damaging the vascular endothelium to predispose to ulcer formation. In certain embodiments a method of treating a chronic wound is provided wherein the chronic wound is characterized by venous ulcers or ulcerations due to malfunction of the venous valve and the associated vascular disease. In certain embodiments a method of treating a chronic wound is provided wherein the chronic wound is characterized by venous stasis ulcers or ulcerations due to chronic passive venous congestion of the lower extremities and/or the resulting local hypoxia.

Exemplary chronic wounds may include “diabetic ulcers.” Diabetic patients are prone to ulcerations, including foot ulcerations, due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. It is not uncommon to have a patient with neuropathy notice that the ulcer “just appeared” when, in fact, the ulcer has been present for quite some time. For patients of neuropathy, strict glucose control has been shown to slow the progression of the disease. Charcot foot deformity may also occur as a result of decreased sensation. People with “normal” feeling in their feet have the ability to sense automatically when too much pressure is being placed on an area of the foot. Once identified, our bodies instinctively shift position to relieve this stress. A patient with advanced neuropathy loses this ability to sense the sustained pressure insult, as a result, tissue ischemia and necrosis may occur leading to for example, plantar ulcerations. Additionally, microfractures in the bones of the foot, if unnoticed and untreated, may result in disfigurement, chronic swelling and additional bony prominences. Microvascular disease is one of the significant complications for diabetics that may also lead to ulcerations. In certain embodiments a method of treating a chronic wound is provided wherein the chronic wound is characterized by diabetic foot ulcers and/or ulcerations due to both neurologic and vascular complications of diabetes.

Exemplary chronic wounds can include “traumatic ulcers.” Formation of exemplary traumatic ulcers may occur as a result of traumatic injuries to the body. These injuries include, for example, compromises to the arterial, venous or lymphatic systems; changes to the bony architecture of the skeleton; loss of tissue layers-epidermis, dermis, subcutaneous soft tissue, muscle or bone; damage to body parts or organs and loss of body parts or organs. In certain embodiments, a method of treating a chronic wound is provided wherein the chronic wound is characterized by ulcerations associated with traumatic injuries to the body.

Exemplary chronic wounds can include “burn ulcers” including for example, ulceration that occur as a result of a burn injury, including 1st degree burn (i.e., superficial, reddened area of skin); 2nd degree burn (a blistered injury site which may heal spontaneously after the blister fluid has been removed); 3rd degree burn (burn through the entire skin and usually require surgical intervention for wound healing); scalding (may occur from scalding hot water, grease or radiator fluid); thermal (may occur from flames, usually deep burns); chemical (may come from acid and alkali, usually deep burns); electrical (either low voltage around a house or high voltage at work); explosion flash (usually superficial injuries); and contact burns (usually deep and may occur from muffler tail pipes, hot irons and stoves). In certain embodiments, a method of treating a chronic wound is provided wherein the chronic wound is characterized by ulcerations associated with burn injuries to the body.

Exemplary chronic wounds can include “vasculitic ulcers.” Vasculitic ulcers also occur on the lower extremities and are painful, sharply marginated lesions, which may have associated palpable purpuras and hemorrhagic bullae. The collagen diseases, septicemias, and a variety of hematological disorders (e.g., thrombocytopenia, dysproteinemia) may be the cause of this severe, acute condition.

Exemplary chronic wounds can include pyoderma gangrenosum. Pyoderma gangrenosum occurs as single or multiple, very tender ulcers of the lower legs. A deep red to purple, undermined border surrounds the purulent central defect. Biopsy typically fails to reveal a vasculitis. In half the patients it is associated with a systemic disease such as ulcerative colitis, regional ileitis, or leukemia. In certain embodiments, a method of treating a chronic wound is provided wherein the chronic wound is characterized by ulcerations associated with pyoderma gangrenosum.

Exemplary chronic wounds can include ocular ulcers, including corneal ulcers or indolent ulcers. Also included are persistent epithelial defects. These may occur in humans and also in sport animals (such as horses) and pet animals (including dogs).

Exemplary chronic wounds can include infectious ulcers. Infectious ulcers follow direct inoculation with a variety of organisms and may be associated with significant regional adenopathy. Mycobacteria infection, anthrax, diphtheria, blastomyosis, sporotrichosis, tularemia, and cat-scratch fever are examples. The genital ulcers of primary syphilis are typically nontender with a clean, firm base. Those of chancroid and granuloma inguinale tend to be ragged, dirty, and more extravagant lesions. In certain embodiments, a method of treating a chronic wound is provided wherein the chronic wound is characterized by ulcerations associated with infection.

As used herein, the term “dehiscent wound” refers to a wound, usually a surgical wound, which has ruptured or split open. In certain embodiments, a method of treating a wound that does not heal at the expected rate is provided wherein the wound is characterized by dehiscence.

In addition to the definition previously provided, the term “wound” may also include for example, injuries to the skin and subcutaneous tissue initiated in different ways (e.g., pressure sores from extended bed rest and wounds induced by trauma) and with varying characteristics.

### Anti-Connexin Agents

Anti-connexin agents, including anti-connexin 26 agents and anti-connexin 43 agents, of the invention described herein are capable of modulating or affecting the transport of molecules into and out of cells (e.g., blocking or inhibiting or downregulating). Thus, certain anti-connexin agents described herein modulate cellular communication (e.g., cell to cell) or extracellular communication (e.g., inside the cell to outside the cell and/or vice versa). Certain anti-connexin agents are gap junction modulation agents. Certain anti-connexin agents modulate or effect transmission of molecules between the cell cytoplasm and the periplasmic or extracellular space. Such anti-connexin agents are generally targeted to connexins and/or connexin hemichannels (connexons). Hemichannels and resulting gap junctions that comprise connexins are independently involved in the release or exchange of small molecules between the cell cytoplasm and an extracellular space or tissue in the case of open hemichannels, and between the cytoplasm of adjoining cell in the case of open gap junctions. Thus, an anti-connexin agents provided herein may directly or indirectly reduce coupling and communication between cells or reduce or block communication (or the transmission of molecules) between a cell and extracellular space or tissue, and the modulation of transport of molecules from a cell into an extracellular space or tissue (or from an extracellular space or tissue into a cell) or between adjoining cells is within the scope of anti-connexin agents and embodiments of the invention. Preferably, the connexin is connexin 43 and/or connexin 26. Other connexins are included and noted herein.

Any anti-connexin agent, including any including anti-connexin 26 agent and/or connexin 43 agent, for example, that is capable of eliciting a desired inhibition of the passage (e.g., transport) of molecules through a gap junction or connexin hemichannel may be used in embodiments of the invention. Any anti-connexin agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such anti-connexin agents may modulate the passage of molecules through a gap junction or connexin hemichannel with or without gap junction uncoupling (blocking the transport of molecules through gap junctions). Such compounds include, for example, proteins and polypeptides, polynucleotides, and other organic compounds, and they may, for example block the function or expression of a gap junction or a hemichannel in whole or in part, or downregulate the production of a connexin in whole or in part. Certain gap junction inhibitors are listed in Evans and Boitano, Biochem. Soc. Trans. 29: 606-612 (2001). Other compounds include connexin phosphorylation compounds that close gap junctions and/or hemichannels, in whole or in part, and connexin carboxy-terminal polypeptides. Preferably, the connexin is connexin 26.

Certain anti-connexin agents provide downregulation of connexin expression (for example, by downregulation of mRNA transcription or translation) or otherwise decrease or inhibit the activity of a connexin protein, a connexin hemichannel or a gap junction. In the case of downregulation, this will have the effect of reducing direct cell-cell communication by gap junctions, or exposure of cell cytoplasm to the extracellular space by hemichannels, at the site at which connexin expression is downregulated. Anti-connexin 26 agents are preferred.

Examples of anti-connexin agents include agents that decrease or inhibit expression or function of connexin mRNA and/or protein or that decrease activity, expression or formation of a connexin, a connexin hemichannel or a gap junction. Anti-connexin agents include anti-connexin polynucleotides, such as antisense polynucleotides and other polynucleotides (such as polynucleotides having siRNA or ribozyme functionalities), as well as antibodies and binding fragments thereof, and peptides and polypeptides, including peptidomimetics and peptide analogs that modulate hemichannel or gap junction activity or function. Anti-connexin 26 agents are preferred.

### Anti-Connexin Polynucleotides

Anti-connexin polynucleotides include connexin antisense polynucleotides as well as polynucleotides which have functionalities which enable them to downregulate connexin expression. Other suitable anti-connexin polynucleotides include RNAi polynucleotides and siRNA polynucleotides. Anti-connexin 26 and anti-connexin 43 polynucleotides, for example, are preferred in certain embodiments.

Synthesis of synthetic antisense polynucleotides and other anti-connexin polynucleotides such as RNAi, siRNA, and ribozyme polynucleotides as well as polynucleotides having modified and mixed backbones is known to those of skill in the art. See, e.g., Stein and Krieg (eds), Applied Antisense Oligonucleotide Technology, 1998 (Wiley-Liss). Methods of synthesizing antibodies and binding fragments as well as peptides and polypeptides, including peptidomimetics and peptide analogs are known to those of skill in the art. See, e.g., Lihu, et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically, the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA, preferably connexin 26 mRNA and connexin 43 mRNA, for example, in certain embodiments. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically, the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides that have sufficient complementarity to form a duplex having a melting temperature of greater than about 20° C., 30° C., or 40° C. under physiological conditions are particularly suitable for use in the present invention.

Thus, the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain embodiments, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000, or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 26 coding sequence shown in Table 8.

Some connexin proteins are, however, more ubiquitous than others in terms of distribution in tissue. Polynucleotides targeted to connexin 26 and connexin 43, for example, are particularly suitable for use in the present invention. In other embodiments other connexins are targeted in conjunction with targeting connexin 26, e.g., connexin 43, and/or cadherin and/or β-catenin.

Anti-connexin polynucleotides include connexin antisense polynucleotides as well as polynucleotides which have functionalities which enable them to downregulate connexin expression. Other suitable anti-connexin polynucleotides include RNAi polynucleotides and SiRNA polynucleotides.

In one preferred aspect, the antisense polynucleotides are targeted to the mRNA of two connexin proteins. Most preferably, connexin 26 and 43. In another aspect, connexin protein is connexin 30, 31.1, 32, 37, 40 or 45. In other embodiments, the connexin protein is connexin 30.3, 31, 40.1, or 46.6.

It is also contemplated that polynucleotides targeted to separate connexin proteins be used in combination (for example 1, 2, 3, 4, or more different connexins may be targeted). For example, polynucleotides targeted to connexin 26 and 43, and one or more other members of the connexin family (such as connexin 30, 30.3, 31.1, 32, 36, 37, 40, 40.1, 45, and 46.6) can be used in combination. Thus, the antisense polynucleotides may be part of compositions that may comprise polynucleotides to more than one connexin protein. Preferably, one of the connexin proteins to which polynucleotides are directed is connexin 26 and another to connexin 43. Other connexin proteins to which oligodeoxynucleotides are directed may include or exclude, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin or may target 1, 2, 3, or more different connexins. Specific polynucleotides will generally target sequences in the connexin gene or mRNA that are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30-mer polynucleotide has been found to be particularly suitable.

Many embodiments of the invention are described with reference to oligodeoxynucleotides. However, it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates, and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ TGT ATT GGG | (connexin 26) | (SEQ. ID. NO: 1) |
| ACA AGG CCA GG 3′ | | |
| 5′ ATC TCT TCG | (connexin 26) | (SEQ. ID. NO: 2) |
| ATG TCC TTA AA 3′ | | |
| 5′ TCC TGA GCA | (connexin 26) | (SEQ. ID. NO: 3) |
| ATA CCT AAC GAA | | |
| CAA ATA 3′ | | |
| 5′ GTA ATT GCG | (connexin 43) | (SEQ. ID. NO: 4) |
| GCA AGA AGA ATT | | |
| GTT TCT GTC 3′ | | |
| 5′ GTA ATT GCG | (connexin 43) | (SEQ. ID. NO: 5) |
| GCA GGA GGA ATT | | |
| GTT TCT GTC 3′ | | |
| 5′ GGC AAG AGA | (connexin 43) | (SEQ. ID. NO: 6) |
| CAC CAA AGA CAC | | |
| TAC CAG CAT 3′ | | |
| 5′ CAT CTC CTT | (connexin 37) | (SEQ. ID. NO: 7) |
| GGT GCT CAA CC 3′ | | |
| 5′ CTG AAG TCG | (connexin 37) | (SEQ. ID. NO: 8) |
| ACT TGG CTT GG 3′ | | |
| 5′ CTC AGA TAG | (connexin 30) | (SEQ. ID. NO: 9) |
| TGG CCA GAA | | |
| TGC 3′ | | |
| 5′ TTG TCC AGG | (connexin 30) | (SEQ. ID. NO: 10) |
| TGA CTC CAA GG 3′ | | |
| 5′ CGT CCG AGC | (connexin 31.1) | (SEQ. ID. NO: 11) |
| CCA GAA AGA TGA | | |
| GGT C 3′ | | |
| 5′ AGA GGC GCA | (connexin 31.1) | (SEQ. ID. NO: 12) |
| CGT GAG ACA C 3′ | | |
| 5′ TGA AGA CAA | (connexin 31.1) | (SEQ. ID. NO: 13) |
| TGA AGA TGT T 3′ | | |
| 5′ TTT CTT TTC | (connexin 32) | (SEQ. ID. NO: 14) |
| TAT GTG CTG TTG | | |
| GTG A 3′ | | |

Suitable polynucleotides for the preparation of the combined polynucleotide compositions described herein include, for example, polynucleotides to connexin Cx26 and connexin 43, and polynucleotides for connexins 26, 30, 31.1, 32, and 37 as described in Table 1 above, for example.

Although the precise sequence of the antisense polynucleotide used in the invention will depend upon the target connexin protein, for connexin 26, antisense polynucleotides having the following sequences have been found to be particularly suitable:

(SEQ. ID. NO: 1)
TGTATTGGGACAAGGCCAGG;
and/or
(SEQ. ID. NO: 2)
ATCTCTTCGATGTCCTTAAA.

For example, suitable antisense polynucleotides for connexins 31.1, 32, and 43 have the following sequences, or any of the sequences noted herein:

(connexin 31.1)
(SEQ. ID. NO: 11)
5′ CGT CCG AGC CCA GAA AGA TGA GGT C;
(connexin 32)
(SEQ. ID. NO: 14)
5′ TTT CTT TTC TAT GTG CTG TTG GTG A;
and
(connexin 43)
(SEQ. ID. NO: 4)
GTA ATT GCG GCA AGA AGA ATT GTT TCT GTC.

Other connexin antisense polynucleotide sequences useful according to the methods of the present invention include:

(connexin 37)
(SEQ. ID. NO: 7)
5′ CAT CTC CTT GGT GCT CAA CC 3′;
(connexin 37)
(SEQ. ID. NO: 8)
5′ CTG AAG TCG ACT TGG CTT GG 3′;
(connexin 30)
(SEQ. ID.NO: 9)
5′ CTC AGA TAG TGG CCA GAA TGC 3′;
(connexin 30)
(SEQ. ID. NO: 10)
5′ TTG TCC AGG TGA CTC CAA GG 3′;
(connexin 31.1)
(SEQ. ID. NO: 12)
5′ AGA GGC GCA CGT GAG ACA C 3′;
and
(connexin 31.1)
(SEQ. ID. NO: 13)
5′ TGA AGA CAA TGA AGA TGT T 3′.

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer to make synthetic polynucleotides, oligonucleotides and ODN's.

### Other Agents, Including Other Polynucleotide Agents

Any anti-cadherin agent and any anti-catenin agent, including any including anti-β-catenin agent, for example, that is capable of eliciting desired cell migration in a wound may be used in embodiments of the invention. They include anti-cadherin and any anti-catenin polynucleotides, similar to those described for anti-connexin polynucleotides, below and herein.

### Polynucleotide Homologues

Homology and homologues are discussed herein (for example, the polynucleotide may be a homologue of a complement to a sequence in connexin mRNA). Such a polynucleotide typically has at least about 70% homology (sequence identity), preferably at least about 80%, at least about 90%, at least about 95%, at least about 97%, or at least about 99% homology or sequence identity with the relevant sequence, for example over a region of at least about 15, at least about 20, at least about 40, at least about 100 more contiguous nucleotides (of the homologous sequence).

Homology (sequence identity) may be calculated based on any method in the art. For example, the UWGCG Package provides the BESTFIT program that can be used to calculate homology (for example, used on its default settings) (Devereux, et al. (1984), Nucleic Acids Research 12, p 387-395). The PILEUP and BLAST algorithms can be used to calculate homology or line up sequences (typically on their default settings), for example as described in Altschul S. F. (1993), J Mol Evol 36: 290-300; Altschul, et al. (1990), J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul, et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T, and X determine the sensitivity and speed of the alignment. The BLAST program uses as defaults a word length (W), the BLOSUM62 scoring matrix (see Henikoff and Henikoff (1992), Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993), Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence (or sequence having substantial sequence identity) typically differs from the relevant sequence by at least about (or by no more than about) 2, 5, 10, 15, 20, more mutations or differences (which may be substitutions, deletions, or insertions). These sequence differences may be measured across any of the regions mentioned above in relation to calculating homology or sequence identity.

The homologous sequence typically hybridizes selectively to the original sequence at a level significantly above background. Selective hybridization is typically achieved using conditions of medium to high stringency (for example, 0.03M sodium chloride and 0.03M sodium citrate at from about 50° C. to about 60° C.). However, such hybridization may be carried out under any suitable conditions known in the art (see Sambrook, et al. (1989), Molecular Cloning: A Laboratory Manual). For example, if high stringency is required, suitable conditions include 0.2×SSC at 60° C. If lower stringency is required, suitable conditions include 2×SSC at 60° C.

### Peptide and Polypeptide Anti-Connexin Agents

Binding proteins, including peptides, peptidomimetics, antibodies, antibody fragments, and the like, are also suitable modulators of gap junctions and hemichannels.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments that are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g., connexin, hemichannel, or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example, those described by Scatchard, et al., 1949 Ann. N.Y. Acad. Sci. 51: 660.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough, D. A., J Cell Biol 107: 1817-1824 (1988); Meyer, R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, et al., Am J. Physiol. Lung Cell Mol. Physiol. 279: L619-L622 (2000); Evans and Boitano, S. Biochem. Soc. Trans. 29: 606-612; and De Vriese, et al. Kidney Int. 61: 177-185 (2001). Short peptides corresponding to sequences within the extracellular loops of connexins were said to inhibit intercellular communication. Boitano and Evans, Am J Physiol Lung Cell Mol Physiol 279: L623-L630 (2000). The use of peptides as inhibitors of cell-cell channel formation produced by connexin (Cx) 32 expressed in paired Xenopus oocytes has also been reported. Dahl, et al., Biophys J 67: 1816-1822 (1994). Berthoud and Seul, summarized some of these results. Am J., Physiol. Lung Cell Mol. Physiol. 279: L619-L622 (2000).

Also, suitable anti-connexin agents comprise a peptide comprising an amino acid sequence corresponding to a portion of a transmembrane region of a connexin 26. Anti-connexin agents include peptides having an amino acid sequence that comprises about 5 to 20 contiguous amino acids of SEQ. ID. NO:15, peptides having an amino acid sequence that comprises about 8 to 15 contiguous amino acids of SEQ. ID. NO:15, or peptides having an amino acid sequence that comprises about 11 to 13 contiguous amino acids of SEQ. ID. NO:15. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:15. Other anti-connexin agents comprise the extracellular domains of connexin 26 corresponding to the amino acids at positions 41-75 and 155-192 of SEQ. ID. NO:15. Anti-connexin agents include peptides described herein which have an amino acid sequence corresponding to the regions, or comprising a portion of the region at positions 41-75 and 155-192 of SEQ. ID. NO:15. The peptides need not have an amino acid sequence identical to those portions of SEQ. ID. NO:15, and conservative amino acid changes may be made such that the peptides retain binding activity or functional activity. Alternatively, peptides may target regions of the connexin protein other than the extracellular domains (e.g., the portions of SEQ. ID. NO:15 not corresponding to positions 41-75 and 155-192). Other anti-connexin agents may comprise a portion of the C-terminal domain of connexin 26.

Anti-connexin agents include peptides comprising an amino acid sequence corresponding to a transmembrane region (e.g., 1^{st} to 4^{th}) of a connexin (e.g., connexin 45, 43, 26, 30, 31.1, and 37). Anti-connexin agents may comprise a peptide comprising an amino acid sequence corresponding to a portion of a transmembrane region of a connexin 45. Anti-connexin agents include a peptide having an amino acid sequence that comprises about 5 to 20 contiguous amino acids of SEQ. ID. NO:16, a peptide having an amino acid sequence that comprises about 8 to 15 contiguous amino acids of SEQ. ID. NO:16, or a peptide having an amino acid sequence that comprises about 11 to 13 contiguous amino acids of SEQ. ID. NO:16. Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:16. In certain anti-connexin agents provided herein, the extracellular domains of connexin 45 corresponding to the amino acids at positions 46-75 and 199-228 of SEQ ID NO:16 may be used to develop the particular peptide sequences. Certain peptides described herein have an amino acid sequence corresponding to the regions at positions 46-75 and 199-228 of SEQ. ID. NO:16. The peptides need not have an amino acid sequence identical to those portions of SEQ. ID. NO:16, and conservative amino acid changes may be made such that the peptides retain binding activity or functional activity. Alternatively, the peptide may target regions of the connexin protein other than the extracellular domains (e.g., the portions of SEQ. ID. NO:16 not corresponding to positions 46-75 and 199-228).

Also, suitable anti-connexin agents comprise a peptide comprising an amino acid sequence corresponding to a portion of a transmembrane region of a connexin 43. Anti-connexin agents include peptides having an amino acid sequence that comprises about 5 to 40, 5 to 30, or 5 to 20 contiguous amino acids of SEQ. ID. NO:17, peptides having an amino acid sequence that comprises about 8 to 15 contiguous amino acids of SEQ. ID. NO:17, or peptides having an amino acid sequence that comprises about 11 to 13 contiguous amino acids of SEQ. ID. NO:17. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:17. Other anti-connexin agents comprise the extracellular domains of connexin 43 corresponding to the amino acids at positions 37-76 and 178-208 of SEQ. ID. NO:17. Anti-connexin agents include peptides described herein which have an amino acid sequence corresponding to the regions, or compromising a portion of the region, at positions 37-76 and 178-208 of SEQ. ID. NO:17. The peptides need not have an amino acid sequence identical to those portions of SEQ. ID. NO:17, and conservative amino acid changes may be made such that the peptides retain binding activity or functional activity. Alternatively, peptides may target regions of the connexin protein other than the extracellular domains (e.g., the portions of SEQ. ID. NO:17 not corresponding to positions 37-76 and 178-208).

| Connexin 26 | |
|---|---|
| (SEQ ID NO: 15) | |
| MDWGTLQTIL GGVNKHSTSI GKIWLTVLFI FRIMILVVAA KEVWGDEQAD | 50 |
| FVCNTLQPGC KNVCYDHYFP ISHIRLWALQ LIFVSTPALL VAMHVAYRRH | 100 |
| EKKRKFIKGE IKSEFKDIEE IKTQKVRIEG SLWWTYTSSI FFRVIFEAAF | 150 |
| MYVFYVMYDG FSMQRLVKCN AWPCPNTVDC FVSRPTEKTV FTVFMIAVSG | 200 |
| ICILLNVTEL CYLLIRYCSG KSKKPV | 226 |

```
Connexin 45
(SEQ ID NO: 16)
Met Ser Trp Ser Phe Leu Thr Arg Leu Leu Glu Glu Ile His Asn His
1 5 10 15
Ser Thr Phe Val Gly Lys Ile Trp Leu Thr Val Leu Ile Val Phe Arg
                        20 25 30
Ile Val Leu Thr Ala Val Gly Gly Glu Ser Ile Tyr Tyr Asp Glu Gln
                35 40 45
Ser Lys Phe Val Cys Asn Thr Glu Gln Pro Gly Cys Glu Asn Val Cys
        50 55 60
Tyr Asp Ala Phe Ala Pro Leu Ser His Val Arg Phe Trp Val Phe Gln
65 70 75 80
Ile Ile Leu Val Ala Thr Pro Ser Val Met Tyr Leu Gly Tyr Ala Ile
                                85 90 95
His Lys Ile Ala Lys Met Glu His Gly Glu Ala Asp Lys Lys Ala Ala
                        100 105 110
Arg Ser Lys Pro Tyr Ala Met Arg Trp Lys Gln His Arg Ala Leu Glu
                115 120 125
Glu Thr Glu Glu Asp Asn Glu Glu Asp Pro Met Met Tyr Pro Glu Met
        130 135 140
Glu Leu Glu Ser Asp Lys Glu Asn Lys Glu Gln Ser Gln Pro Lys Pro
145 150 155 160
Lys His Asp Gly Arg Arg Arg Ile Arg Glu Asp Gly Leu Met Lys Ile
                                165 170 175
Tyr Val Leu Gln Leu Leu Ala Arg Thr Val Phe Glu Val Gly Phe Leu
                        180 185 190
Ile Gly Gln Tyr Phe Leu Tyr Gly Phe Gln Val His Pro Phe Tyr Val
                195 200 205
Cys Ser Arg Leu Pro Cys Pro His Lys Ile Asp Cys Phe Ile Ser Arg
        210 215 220
Pro Thr Glu Lys Thr Ile Phe Leu Leu Ile Met Tyr Gly Val Thr Gly
225 230 235 240
Leu Cys Leu Leu Leu Asn Ile Trp Glu Met Leu His Leu Gly Phe Gly
                                245 250 255
Thr Ile Arg Asp Ser Leu Asn Ser Lys Arg Arg Glu Leu Glu Asp Pro
                        260 265 270
Gly Ala Tyr Asn Tyr Pro Phe Thr Trp Asn Thr Pro Ser Ala Pro Pro
                275 280 285
Gly Tyr Asn Ile Ala Val Lys Pro Asp Gln Ile Gln Tyr Thr Glu Leu
        290 295 300
Ser Asn Ala Lys Ile Ala Tyr Lys Gln Asn Lys Ala Asn Thr Ala Gln
305 310 315 320
Glu Gln Gln Tyr Gly Ser His Glu Glu Asn Leu Pro Ala Asp Leu Glu
                                325 330 335
Ala Leu Gln Arg Glu Ile Arg Met Ala Gln Glu Arg Leu Asp Leu Ala
                        340 345 350
Val Gln Ala Tyr Ser His Gln Asn Asn Pro His Gly Pro Arg Glu Lys
                355 360 365
Lys Ala Lys Val Gly Ser Lys Ala Gly Ser Asn Lys Ser Thr Ala Ser
        370 375 380
Ser Lys Ser Gly Asp Gly Lys Asn Ser Val Trp Ile
385 390 395
Connexin 43
(SEQ ID NO: 17)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Glu Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                      180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

The anti-connexin peptides for use in conjunction with anti-Cx26 agents may comprise sequences corresponding to a portion of the connexin extracellular domains with conservative amino acid substitutions such that peptides are functionally active anti-connexin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 mRNA are shown below in Table 2.

TABLE 2
Peptidic Inhibitors of Intercellular
Communication (Cx43)

| | Location on | |
|---|---|---|
| Peptide Sequence | Cx43 Protein | SEQ ID NO. |
| FEVAFLLIQWI | M3 & E2 | (SEQ. ID. NO: 18) |
| LLIQWYIGFSL | E2 | (SEQ. ID. NO: 19) |
| SLSAVYTCKRDPCPHQ | E2 | (SEQ. ID. NO: 20) |
| VDCFLSRPTEKT | E2 | (SEQ. ID. NO: 21) |
| SRPTEKTIFII | E2 & M4 | (SEQ. ID. NO: 22) |
| LGTAVESAWGDEQ | M1 & El | (SEQ. ID. NO: 23) |
| QSAFRCNTQQPG | E1 | (SEQ. ID. NO: 24) |
| QQPGCENVCYDK | E1 | (SEQ. ID. NO: 25) |
| VCYDKSFPISHVR | E1 | (SEQ. ID. NO: 26) |

Table 3 provides additional exemplary connexin peptides used in inhibiting hemichannel or gap junction function. In other embodiments, conservative amino acid changes are made to the peptides or fragments thereof.

TABLE 3
Additional Peptidic Inhibitors of Intercellular Communication

| Connexin | Location | Amino Acid Sequence | SEQ ID NO: |
|---|---|---|---|
| Cx32 | E1 39-77 | AAESVWGDEIKSSFICNTL | (SEQ. ID. NO: 27) |
| | | QPGCNSVCYDHFFPISHVR | |
| Cx32 | E1 41-52 | ESVWGDEKSSFI | (SEQ. ID. NO: 28) |
| Cx32 | E1 52-63 | ICNTLQPGCNSV | (SEQ. ID. NO: 29) |
| Cx32 | E1 62-73 | SVCYDHFFPISH | (SEQ. ID. NO: 30) |
| Cx32 | E2 64-188 | RLVKCEAFPCPNTVDCFVSRPTEKT | (SEQ. ID. NO: 31) |
| Cx32 | E2 166-177 | VKCEAFPCPNTV | (SEQ. ID. NO: 32) |
| Cx32 | E2 177-188 | VDCFVSRPTEKT | (SEQ. ID. NO: 33) |
| Cx32 | E1 63-75 | VCYDHFFPISHVR | (SEQ. ID. NO: 34) |
| Cx32 | E1 45-59 | VWGDEKSSFICNTLQPGY | (SEQ. ID. NO: 35) |
| Cx32 | E1 46-59 | DEKSSFICNTLQPGY | (SEQ. ID. NO: 36) |
| Cx26 | E2 | SRPTEKTVFTV | (SEQ .ID .NO: 37) |
| Cx26 | E2 | VDCFVSRPTEKT | (SEQ. ID. NO: 33) |
| Cx32 | E2 182-192 | SRPTEKTVFTV | (SEQ. ID. NO: 37) |
| Cx32/Cx43/ | E2 182-188/ | SRPTEKT | (SEQ. ID. NO: 38) |
| Cx26, Cx30, | 201-207 | | |
| Cx30.3, | | | |
| Cx36, Cx37, | | | |
| Cx46, | | | |
| Cx46.6, | | | |
| Cx45 | | | |
| Cx32 | E1 52-63 | ICNTLQPGCNSV | (SEQ. ID. NO: 39) |
| Cx40 | E2 177-192 | FLDTLHVCRRSPCPHP | (SEQ. ID. NO: 40) |
| Cx43 | E2 188-205 | KRDPCHQVDCFLSRPTEK | (SEQ. ID. NO: 41) |

Table 4 provides the extracellular loops for connexin family members that are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 4
Extracellular loops for various connexin family members

| Name | Amino Acid Sequence | SEQ ID. NO: |
|---|---|---|
| huCx26 | KEVWGDEQADFVCNTLQPGCKNVCYDHYFPISHIR | (SEQ. ID. NO: 42) |
| huCx30 | QEVWGDEQEDFVCNTLQPGCKNVCYDHFFPVSHIR | (SEQ. ID. NO: 43) |
| huCx30.3 | EEVWDDEQKDFVCNTKQPGCPNVCYDEFFPVSHVR | (SEQ. ID. NO: 44) |
| huCx31 | ERVWGDEQKDFDCNTKQPGCTNVCYDNYFPISNIR | (SEQ. ID. NO: 45) |
| huCx31.1 | ERVWSDDHKDFDCNTRQPGCSNVCFDEFFPVSHVR | (SEQ. ID. NO: 46) |
| huCx32 | ESVWGDEKSSFICNTLQPGCNSVCYDQFFPISHVR | (SEQ. ID. NO: 47) |
| huCx36 | ESVWGDEQSDFECNTAQPGCTNVCYDQAFPISHIR | (SEQ. ID. NO: 48) |
| huCx37 | ESVWGDEQSDFECNTAQPGCTNVCYDQAFPISHIR | (SEQ. ID. NO: 49) |
| huCx40.1 | RPVYQDEQERFVCNTLQPGCANVCYDVFSPVSHLR | (SEQ. ID. NO: 50) |
| huCx43 | ESAWGDEQSAFRCNTQQPGCENVCYDKSFPISHVR | (SEQ. ID. NO: 51) |
| huCx46 | EDVWGDEQSDFTCNTQQPGCBNVCYBRAFPISHIR | (SEQ. ID. NO: 52) |
| huCx46.6 | EAIYSDEQAKFTCNTRQPGCDNVCYDAFAPLSHVR | (SEQ. ID. NO: 53) |
| huCx40 | ESSWGDEQADFRCDTIQPGCQNVCTDQAFPISHIR | (SEQ. ID. NO: 54) |
| huCx45 | GESIYYDEQSKFVCNTEQPGCENVCYDAFAPLSHVR | (SEQ. ID. NO: 55) |
| huCx26 | MYVFYVMYDGFSMQRLVKCNAWPCPNTVDCFVSRPTEKT | (SEQ. ID. NO: 56) |
| huCx30 | MYVFYFLYNGYHLPWVLKCGIDPCPNLVDCFISRPTEKT | (SEQ. ID. NO: 57) |
| huCx30.3 | LYIFHRLYKDYDMPRVVACSVEPCPHTVDCYISRPTEKK | (SEQ. ID. NO: 58) |
| huCx31 | LYLLHTLWHGFNMPRLVQCANVAPCPNIVDCYIARPTEKK | (SEQ. ID. NO: 59) |
| huCx31.1 | LYVFHSFYPKYILPPVVKCHADPCPNIVDCFISKPSEKN | (SEQ. ID. NO: 60) |
| huCx32 | MYVFYLLYPGYAMVRLVKCDVYPCPNTVDCFVSRPTEKT | (SEQ. ID. NO: 61) |
| huCx36 | LYGWTMEPVFVCQRAPCPYLVDCFVSRPTEKT | (SEQ. ID. NO: 62) |
| huCx37 | LYGWTMEPVFVCQRAPCPYLVDCFVSRPTEKT | (SEQ. ID. NO: 63) |
| huCx40.1 | GALHYFLFGFLAPKKFPCTRPPCTGVVDCYVSRPTSKS | (SEQ. ID. NO: 64) |
| huCx43 | LLIQWYIYGFSLSAVYTCKRDPCPHQVDCFLSRPTEKT | (SEQ. ID. NO: 65) |
| huCx46 | IAGQYFLYGFELKPLYRCDRWPCPNTVDCFISRPTEKT | (SEQ. ID. NO: 66) |
| huCx46.6 | LVGQYLLYGFEVRPFFPCSRQPCPHVVDCFVSRPTEKT | (SEQ. ID. NO: 67) |
| huCx40 | IVGQYFIYGIFLTTLHVCRRSPCPHPVNCYVSRPTEKN | (SEQ. ID. NO: 68) |
| huCx45 | LIGQYFLYGFQVHPFYVCSRLPCHPKIDCFISRPTEKT | (SEQ. ID. NO: 69) |

Table 5 provides the extracellular domain for connexin family members that may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 5
Extracellular domains

| Name | Amino Acid Sequence | SEQ. ID. NO: |
|---|---|---|
| Peptide | VDCFLSRPTEKT | (SEQ. ID. NO: 21) |
| Peptide | SRPTEKTIFII | (SEQ. ID. NO: 22) |
| huCx43 | LLIQWYIYGFSLSAVYTCKRDPCPHQVDCFLSRPTEKTIFII | (SEQ. ID. NO: 70) |
| huCx26 | MYVFYVMYDGFSMQRLVKCNAWPCPNTVDCFVSRPTEKTVFTV | (SEQ. ID. NO: 71) |
| huCx30 | YVFYFLYNGYHLPWVLKCGIDPCPNLVDCFISRPTEKTVFTI | (SEQ. ID. NO: 72) |
| huCx30.3 | LYIFHRLYKDYDMPRVVACSVEPCPHTVDCYISRPTEKKVFTY | (SEQ. ID. NO: 73) |
| huCx31 | LYLLHTLWHGFNMPRLVQCANVAPCPNIVDCYIARPTEKKTY | (SEQ. ID. NO: 74) |
| huCx31.1 | LYVFHSFYPKYILPPVVKCHADPCPNIVDCFISKPSEKNIFTL | (SEQ. ID. NO: 75) |
| huCx32 | MYVFYLLYPGYAMVRLVKCDVYPCPNTVDCFVSRPTEKTVFTV | (SEQ. ID. NO: 76) |
| huCx36 | LYGWTMEPVFVCQRAPCPYLVDCFVSRPTEKTIFII | (SEQ. ID. NO: 77) |
| huCx37 | LYGWTMEPVFVCQRAPCPYLVDCFVSRPTEKTIFII | (SEQ. ID. NO: 78) |
| huCx40.1 | GALHYFLFGFLAPKKFPCTRPPCTGVVDCYVSRPTEKSLLML | (SEQ. ID. NO: 79) |
| huCx46 | IAGQYFLYGFELKPLYRCDRWPCPNTVDCFISRPTEKTIFII | (SEQ. ID. NO: 80) |
| huCx46.6 | LVGQYLLYGFEVRPFFPCSRQPCPHVVDCFVSRPTEKTVFLL | (SEQ. ID. NO: 81) |
| huCx40 | IVGQYFIYGIFLTTLHVCRRSPCPHPVNCYSRPTEKNVFIV | (SEQ. ID. NO: 82) |
| huCx45 | LIGQYFLYGFQVHPFYVCSRLPCHPKIDCFISRPTEKTIFLL | (SEQ. ID. NO: 83) |

Table 6 provides peptides inhibitors of connexin 40 shown with reference to the extracellular loops (E1 and E2) of connexin 40. The bold amino acids are directed to the transmembrane regions of connexin 40.

TABLE 6
Cx40 peptide inhibitors

| | Amino Acid Sequence | SEQ. ID. NO. |
|---|---|---|
| E1 | LGTAAESSWGDEQADFRCDTIQPGCQNVCTDQAFPISHIRFWVLQ | (SEQ. ID. NO: 84) |
| | LGTAAESSWGDEQA | (SEQ. ID. NO: 85) |
| | DEQADFRCDTIQP | (SEQ. ID. NO: 86) |
| | TIQPGCQNVCTDQ | (SEQ. ID. NO: 87) |
| | VCTDQAFPISHIR | (SEQ. ID. NO: 88) |
| | AFPISHIRFWVLQ | (SEQ. ID. NO: 89) |
| E2 | MEVGFIVGQYFIYGIFLTTLHVCRRSPCPHPVNCYVSRPTEKNVFIV | (SEQ. ID. NO: 90) |
| | MEVGFIVGQYF | (SEQ. ID. NO: 91) |
| | IVGQYFIYGIFL | (SEQ. ID. NO: 92) |
| | GIFLTTLHVCRRSP | (SEQ. ID. NO: 93) |
| | RRSPCPHPVNCY | (SEQ. ID. NO: 94) |
| | VNCYVSRPTEKN | (SEQ. ID. NO: 95) |
| | SRPTEKNVFIV | (SEQ. ID. NO: 96) |

Table 7 provides peptides inhibitors of connexin 45 shown with reference to the extracellular loops (E1 and E2) of connexin 45. The bold amino acids are directed to the transmembrane regions of connexin 45.

TABLE 7
Cx45 peptide inhibitors

| | Amino Acid Sequence | SEQ ID. NO. |
|---|---|---|
| E1 | LTAVGGESIYYDEQSKFVCNTEQPGCENVCYDAFAPLSHVRFWVFQ | (SEQ. ID. NO: 97) |
| | LTAVGGESIYYDEQS | (SEQ. ID. NO: 98) |
| | DEQSKFVCNTEQP | (SEQ. ID. NO: 99) |
| | TEQPGCENVCYDA | (SEQ. ID. NO: 100) |
| | VCYDAFAPLSHVR | (SEQ. ID. NO: 101) |
| | APLSHVRFWVFQ | (SEQ. ID. NO: 102) |
| E2 | FEVGFLIGQYFLYGFQVHPFYVCSRLPCHPKIDCFISRPTEKTIFLL | (SEQ. ID. NO: 103) |
| | FEVGFLIGQYF | (SEQ. ID. NO: 104) |
| | LIGQYFLYGFQV | (SEQ. ID. NO: 105) |
| | GFQVHPFYVCSRLP | (SEQ. ID. NO: 106) |
| | SRLPCHPKIDCF | (SEQ. ID. NO: 107) |
| | IDCFISRPTEKT | (SEQ. ID. NO: 108) |
| | SRPTEKTIFLL | (SEQ. ID. NO: 109) |

In certain embodiments, it is preferred that certain peptide inhibitors block hemichannels without disrupting existing gap junctions. While not wishing to be bound to any particular theory or mechanism, it is also believed that certain peptidomimetics (e.g., VCYDKSFPISHVR, (SEQ. ID. NO:26) block hemichannels without causing uncoupling of gap junctions (See Leybeart, Cell Commun. Adhes. 10: 251-257 (2003)), or do so in lower dose amounts. A peptide comprising SRPTEKT (SEQ. ID. NO:38) or SRPTEKTIFII (SEQ. ID. NO:22) may also be used, for example to block hemichannels without uncoupling of gap junctions. The peptide SRGGEKNVFIV (SEQ. ID. NO:110) may be used that as a control sequence (DeVriese, et al., Kidney Internat. 61: 177-185 (2002)). Examples of peptide inhibitors for connexin 45 YVCSRLPCHP (SEQ. ID. NO:111), QVHPFYVCSRL (SEQ. ID. NO:112), FEVGFLIGQYFLY (SEQ. ID. NO:113), GQYFLYGFQVHP (SEQ. ID. NO:114), GFQVHPFYVCSR (SEQ. ID. NO:115), AVGGESIYYDEQ (SEQ. ID. NO:116), YDEQSKFVCNTE (SEQ. ID. NO:117), NTEQPGCENVCY (SEQ. ID. NO:118), CYDAFAPLSHVR (SEQ. ID. NO:119), FAPLSHVRFWVF (SEQ. ID. NO:120) and LIGQY (SEQ. ID. NO:121), QVHPF (SEQ. ID. NO:122), YVCSR (SEQ. ID. NO:123), SRLPC (SEQ. ID. NO:124), LPCHP (SEQ. ID. NO:125) and GESIY (SEQ. ID. NO:126), YDEQSK (SEQ. ID. NO:127), SKFVCN (SEQ. ID. NO:128), TEQPGCEN (SEQ. ID. NO:129), VCYDAFAP (SEQ. ID. NO:130), LSHVRFWVFQ (SEQ. ID. NO:131) The peptides may only be 3 amino acids in length, including SRL, PCH, LCP, CHP, IYY, SKF, QPC, VCY, APL, HVR, or longer, for example: LIQYFLYGFQVHPF (SEQ. ID. NO:132), VHPFYCSRLPCHP (SEQ. ID. NO:133), VGGESIYYDEQSKFVCNTEQPG (SEQ. ID. NO:134), TEQPGCENVCYDAFAPLSHVRF (SEQ. ID. NO:135), AFAPLSHVRFWVFQ (SEQ. ID. NO:136).

Peptides comprising connexin C-terminal peptides may also include, for example, peptides comprising from 5 to 40 amino acids of the C-terminal domain of a connexin such as connexin 26, connexin 43 or connexin 31.1, for example about 5 to 40, 5 to 30, or 5 to 20 contiguous amino acids of the C-terminal domain, peptides having an amino acid sequence that comprises about 8 to 15 contiguous amino acids of the C-terminal domain, or peptides having an amino acid sequence that comprises about 11 to 13 contiguous amino acids of a C-terminal domain. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of a C-terminal domain of a connexin such as connexin 26, connexin 43 or connexin 31.1. For example, peptides comprising connexin C-terminal peptides may comprise the following sequences: RPRPDDLEI (SEQ. ID. NO:137), PSSRASSRASSRPRPDDLEI (SEQ. ID. NO:138), RPRPDDLEV (SEQ. ID. NO:139), CSGKSKKPV (SEQ. ID. NO:140), or CYLLIRYCSGKSKKPV (SEQ. ID. NO:141). The peptides may further comprise a connexin peptide from an intracellular portion of a connexin, and a second signalling peptide, such as a cell internalization signal, for example, RQPKIWFPNRRKPWKK (SEQ. ID. NO:142) or NSKKLAAGHELQPLAIVDQRPSSRASSRASS (SEQ. ID. NO:143). The peptide comprising the signalling peptide and the connexin intracellular domain may be, for example, RQPKIWFPNRRKPWKKRPRPDDLEI (SEQ. ID. NO:144) and NSKKLAAGHELQPLAIVDQRPSSRASSRASSRPRPDDLEI (SEQ. ID. NO. 145), RQPKIWFPNRRKPWKKCSGKSKKPV (SEQ ID NO:146).

TABLE 8
Human Connexin 43 DNA Sequence
Table 8A
Human Connexin 43 from GenBank Accession No. M65188 (SEQ. ID. NO: 147)

| | |
|---|---|
| 1 | ggcttttagc gtgaggaaag taccaaacag cagcggagtt ttaaacttta aatagacagg |
| 61 | tctgagtgcc tgaacttgcc ttttcatttt acttcatcct ccaaggagtt caatcacttg |
| 121 | gcgtgacttc actactttta agcaaaagag tggtgcccag gcaacatggg tgactggagc |
| 181 | gccttaggca aactccttga caaggttcaa gcctactcaa ctgctggagg gaaggtgtgg |
| 241 | ctgtcagtac ttttcatttt ccgaatcctg ctgctgggga cagcggttga gtcagcctgg |
| 301 | ggagatgagc agtctgcctt tcgttgtaac actcagcaac ctggttgtga aaatgtctgc |
| 361 | tatgacaagt ctttcccaat ctctcatgtg cgcttctggg tcctgcagat catatttgtg |
| 421 | tctgtaccca cactcttgta cctggctcat gtgttctatg tgatgcgaaa ggaagagaaa |
| 481 | ctgaacaaga aagaggaaga actcaaggtt gcccaaactg atggtgtcaa tgtggacatg |
| 541 | cacttgaagc agattgagat aaagaagttc aagtacggta ttgaagagca tggtaaggtg |
| 601 | aaaatgcgag gggggttgct gcgaacctac atcatcagta tcctcttcaa gtctatcttt |
| 661 | gaggtggcct tcttgctgat ccagtggtac atctatggat tcagcttgag tgctgtttac |
| 721 | acttgcaaaa gagatccctg cccacatcag gtggactgtt tcctctctcg ccccacggag |
| 781 | aaaaccatct tcatcatctt catgctggtg gtgtccttgg tgtccctggc cttgaatatc |
| 841 | attgaactct tctatgtttt cttcaagggc gttaaggatc gggttaaggg aaagagcgac |
| 901 | ccttaccatg cgaccagtgg tgcgctgagc cctgccaaag actgtgggtc tcaaaaatat |
| 961 | gcttatttca atggctgctc ctcaccaacc gctcccctct cgcctatgtc tcctcctggg |
| 1021 | tacaagctgg ttactggcga cagaaacaat tcttcttgcc gcaattacaa caagcaagca |
| 1081 | agtgagcaaa actgggctaa ttacagtgca gaacaaaatc gaatggggca ggcgggaagc |
| 1141 | accatctcta actcccatgc acagcctttt gatttccccg atgataacca gaattctaaa |
| 1201 | aaactagctg ctggacatga attacagcca ctagccattg tggaccagcg accttcaagc |
| 1261 | agagccagca gtcgtgccag cagcagacct cggcctgatg acctggagat ctag |

Table 8B
Human Connexin 43 (SEQ. ID. NO: 148)

| | |
|---|---|
| 1 | atgggtgact ggagcgcctt aggcaaactc cttgacaagg ttcaagccta ctcaactgct |
| 61 | ggagggaaggtgtggctgtc agtacttttc attttccgaatcctgctgct ggggacagcg |
| 121 | gttgagtcagcctggggaga tgagcagtct gcctttcgtt gtaacactca gcaacctggt |
| 181 | tgtgaaaatg tctgctatga caagtctttcccaatctctc atgtgcgctt ctgggtcctg |
| 241 | cagatcatat ttgtgtctgt acccacactcttgtacctgg ctcatgtgttctatgtgatg |
| 301 | cgaaaggaag agaaactgaa caagaaagag gaagaactca aggttgccca aactgatggt |
| 361 | gtcaatgtgg acatgcactt gaagcagatt gagataaagaagttcaagta cggtattgaa |
| 421 | gagcatggta aggtgaaaat gcgagggggg ttgctgcgaa cctacatcat cagtatcctc |
| 481 | ttcaagtcta tctttgaggt ggccttcttg ctgatccagt ggtacatcta tggattcagc |
| 541 | ttgagtgctg tttacacttg caaaagagat ccctgcccac atcaggtgga ctgtttcctc |
| 601 | tctcgcccca cggagaaaac catcttcatc atcttcatgc tggtggtgtc cttggtgtcc |
| 661 | ctggccttga atatcattga actcttctat gttttcttca agggcgttaa ggatcgggtt |
| 721 | aagggaaaga gcgaccctta ccatgcgacc agtggtgcgc tgagccctgc caaagactgt |
| 781 | gggtctcaaa aatatgctta tttcaatggc tgctcctcac caaccgctcc cctctcgcct |
| 841 | atgtctcctc ctgggtacaa gctggttact ggcgacagaa acaattcttc ttgccgcaat |
| 901 | tacaacaagc aagcaagtga gcaaaactgg gctaattaca gtgcagaaca aaatcgaatg |
| 961 | gggcaggcgg gaagcaccat ctctaactcc catgcacagccttttgattt ccccgatgat |
| 1021 | aaccagaatt ctaaaaaactagctgctgga catgaattac agccactagc cattgtggac |
| 1081 | cagcgacctt caagcagagc cagcagtcgtgccagcagca gacctcggcctgatgacctg |
| 1141 | gagatctag |

### Gap Junction Modulation Agents

Certain anti-connexin agents described herein are capable of modulation or affecting the transport of molecules into and out of cells (e.g., blocking or inhibiting). Thus certain gap junction modulation agents described herein modulate cellular communication (e.g., cell to cell). Certain gap junction modulation agents modulate or affect transmission of molecules between the cell cytoplasm and the periplasmic or extracellular space. Such agents are generally targeted to hemichannels (also called connexons), which may be independently involved in the exchange of small molecules between the cell cytoplasm and an extracellular space or tissue. Thus, a compound provided herein may directly or indirectly reduce coupling between cells (via gap junctions) or between a cell and an extracellular space or tissue (via hemichannels), and the modulation of transport of molecules from a cell into an extracellular space is within the scope of certain compounds and embodiments of the invention.

Any molecule that is capable of eliciting a desired inhibition of the passage (e.g., transport) of molecules through a gap junction or hemichannel may be used in embodiments of the invention. Compounds that modulate the passage of molecules through a gap junction or hemichannel are also provided in particular embodiments (e.g., those that modulate the passage of molecules from the cytoplasm of a cell into an extracellular space). Such compounds may modulate the passage of molecules through a gap junction or hemichannel with or without gap junction uncoupling. Such compounds include, for example, binding proteins, polypeptides, and other organic compound that can, for example, block the function or activity of a gap junction or a hemichannel in whole or in part.

As used herein, “gap junction modulation agent” may broadly include those agents or compounds that prevent, decrease or modulate, in whole or in part, the activity, function, or formation of a hemichannel or a gap junction. In certain embodiments, a gap junction modulation agent prevents or decreases, in whole or in part, the function of a hemichannel or a gap junction. In certain embodiments, a gap junction modulation agent induces closure, in whole or in part, of a hemichannel or a gap junction. In other embodiments, a gap junction modulation agent blocks, in whole or in part, a hemichannel or a gap junction. In certain embodiments, a gap junction modulation agent decreases or prevents, in whole or in part, the opening of a hemichannel or gap junction. In certain embodiments, said blocking or closure of a gap junction or hemichannel by a gap junction modulation agent can reduce or inhibit extracellular hemichannel communication by preventing or decreasing the flow of small molecules through an open channel to and from an extracellular or periplasmic space. Peptidomimetics, and gap junction phosphorylation compounds that block hemichannel and/or gap junction opening are presently preferred.

In certain embodiments, a gap junction modulation agent prevents, decreases or alters the activity or function of a hemichannel or a gap junction. As used herein, modification of the gap junction activity or function may include or exclude, for example, the closing of gap junctions, closing of hemichannels, and/or passage of molecules or ions through gap junctions and/or hemichannels.

Exemplary gap junction modulation agents may include or exclude, for example, without limitation, polypeptides (e.g., peptiditomimetics, antibodies, binding fragments thereof, and synthetic constructs), and other gap junction blocking agents, and gap junction protein phosphorylating agents. Exemplary compounds used for closing gap junctions (e.g., phosphorylating connexin 43 tyrosine residue) have been reported in U.S. Pat. No. 7,153,822 to Jensen, et al., U.S. Pat. No. 7,250,397, and assorted patent publications. Exemplary peptides and peptidomimetics are reported in Green et al., WO2006134494. See also Gourdie, et al., see WO2006069181, and Tudor, et al., see WO2003032964. In some embodiments, the gap junction modulation agent can include or exclude tonabersat, for example.

As used herein, “gap junction phosphorylating agent” may include those agents or compounds capable of inducing phosphorylation on connexin amino acid residues in order to induce gap junction or hemichannel closure. Gap junction modulation exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include or exclude, for example, c-Src tyrosine kinase or other G protein-coupled receptor agonists. See Giepmans, B (2001), J. Biol. Chem., Vol. 276, Issue 11, 8544-8549. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Polypeptide compounds, including binding proteins (e.g., antibodies, antibody fragments, and the like), peptides, peptidomimetics, and peptidomimetics, are suitable modulators of gap junctions.

Binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding protein known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g., connexin, connexon, gap junctions, or associated molecules).

Binding proteins will generally have a desired specificity, including but not limited to binding specificity, and desired affinity. Affinity, for example, may be a Ka of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard, et al., (1949) Ann. N.Y. Acad. Sci. 51: 660.

The invention includes use of peptides (including peptidomimetic and peptidomimetics) for modulation of gap junctions and hemichannels. By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough, D. A. (1988), J Cell Biol 107: 1817-1824; Meyer, R. A. (1992), J Cell Biol 119: 179-189.

Peptides or variants thereof, can be synthesized in vitro, e.g., by the solid phase peptide synthetic method or by enzyme-catalyzed peptide synthesis or with the aid of recombinant DNA technology. Solid phase peptide synthetic method is an established and widely used method, which is described in references such as the following: Stewart, et al., (1969), Solid Phase Peptide Synthesis, W. H. Freeman Co., San Francisco; Merrifield, (1963) J. Am. Chem. Soc. 85 2149; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

The extracellular domains of a hemichannel contributed by two adjacent cells “dock” with each other to form complete gap junction channels. Reagents that interfere with the interactions of these extracellular domains can impair cell-to-cell communication, or with hemichannel opening to the extracellular environment.

Gap junction modulation agents include peptides comprising an amino acid sequence corresponding to a transmembrane region (e.g., 1st to 4th) of a connexin (e.g., connexin 45, 43, 26, 30, 31.1, and 37). Gap junction modulation agents including a peptide comprising an amino acid sequence corresponding to a portion of a transmembrane region of a connexin 43 are preferred for use in the present inventions.

Gap junction modulation agents may comprise a peptide comprising an amino acid sequence corresponding to a portion of a transmembrane region of a connexin 45. Gap junction modulation agents include a peptide having an amino acid sequence that comprises about 5 to 20 contiguous amino acids of SEQ. ID. NO:16, a peptide having an amino acid sequence that comprises about 8 to 15 contiguous amino acids of SEQ. ID. NO:16, or a peptide having an amino acid sequence that comprises about 11 to 13 contiguous amino acids of SEQ. ID. NO:16. Other embodiments are directed to an gap junction modulation compound that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:16. In certain gap junction modulation compounds provided herein, the extracellular domains of connexin 45 corresponding to the amino acids at positions 46-75 and 199-228 of SEQ. ID. NO:16 may be used to develop the particular peptide sequences. Certain peptides described herein have an amino acid sequence corresponding to the regions at positions 46-75 and 199-228 of SEQ. ID. NO:16. The peptides need not have an amino acid sequence identical to those portions of SEQ. ID. NO:16, and conservative amino acid changes may be made such that the peptides retain binding activity or functional activity. Alternatively, the peptide may target regions of the connexin protein other than the extracellular domains (e.g., the portions of SEQ. ID. NO:16 not corresponding to positions 46-75 and 199-228).

Also, suitable gap junction modulation agents can include a peptide comprising an amino acid sequence corresponding to a portion of a transmembrane region of a connexin 43. Gap junction modulation agents include peptides having an amino acid sequence that comprises about 5 to 20 contiguous amino acids of SEQ. ID. NO:17, peptides having an amino acid sequence that comprises about 8 to 15 contiguous amino acids of SEQ. ID. NO:17, or peptides having an amino acid sequence that comprises about 11 to 13 contiguous amino acids of SEQ. ID. NO:17. Other gap junction modulation agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ. ID. NO:17. Other gap junction modulation agents comprise the extracellular domains of connexin 43 corresponding to the amino acids at positions 37-76 and 178-208 of SEQ. ID. NO:17. Gap junction modulation agents include peptides described herein which have an amino acid sequence corresponding to the regions at positions 37-76 and 178-208 of SEQ. ID. NO:17. The peptides need not have an amino acid sequence identical to those portions of SEQ. ID. NO:17, and conservative amino acid changes may be made such that the peptides retain binding activity or functional activity. Alternatively, peptides may target regions of the connexin protein other than the extracellular domains (e.g., the portions of SEQ. ID. NO:17 not corresponding to positions 37-76 and 178-208).

Still other anti-connexin agents include connexin carboxy-terminal polypeptides. See Gourdie, et al., WO2006/069181, herein incorporated by reference in its entirety.

### Gap Junction Modifying Agents—Other Anti-Connexin Agents

Gap junction modulation agents, include agents that close or block gap junctions and/or hemichannels or otherwise prevent or decrease cell to cell communication via gap junctions or prevent or decrease cell communication to the extracellular environment via hemichannels. They include agents or compounds that prevent, decrease or inhibit, in whole or in part, the activity, function, or formation of a hemichannel or a gap junction.

In certain embodiments, a gap junction modulation agent induces closure, in whole or in part, of a hemichannel or a gap junction. In other embodiments, a gap junction-modifying agent blocks, in whole or in part, a hemichannel or a gap junction. In certain embodiments, a gap junction-modifying agent decreases or prevents, in whole or in part, the opening of a hemichannel or gap junction.

In certain embodiments, said blocking or closure of a gap junction or hemichannel by a gap junction modifying agent can reduce or inhibit extracellular hemichannel communication by preventing or decreasing the flow of small molecules through an open channel to and from an extracellular or periplasmic space.

Gap junction modifying agents used for closing hemichannels or gap junctions (e.g., phosphorylating connexin 43 tyrosine residues) have been reported in U.S. Pat. No. 7,153,822, U.S. Pat. No. 7,250,397, and assorted patent publications. See also, Gourdie, et al., see WO2006069181, with regard to connexin carboxy-terminal polypeptides that are said to, for example, inhibit ZO-1 protein binding. Gourdie, et al, WO2006069181, describes use of formulations comprising such peptides.

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans, B, J. (2001), Biol. Chem., Vol. 276, Issue 11, 8544-8549. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie, et al., WO2006/069181.

In certain another aspect, gap junction modifying agent may include or exclude, for example, aliphatic alcohols; octanol; heptanol; anesthetics (e.g., halothane), ethane, fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g., arachidonic acid, oleic acid and lipoxygenase metabolites; Fenamates (flufenamic (FFA), niflumic (NFA) and meclofenamic acids (MFA)); Genistein; glycyrrhetinic acid (GA): 18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g., acidifying agents; polyunsaturated fatty acids; fatty acid GJIC inhibitors (e.g., oleic and arachidonic acids); quinidine; quinine; all trans-retinoic acid; tonabersat; and tamoxifen.

### Dosage Forms and Formulations and Administration

A therapeutically effective amount of each of the combination partners in the sustained release delivery devices of the invention may be administered simultaneously, separately or sequentially and in any order. The agents may be administered separately by layer, for example, or as a fixed combination. In certain embodiments, the anti-connexin agent or agents are anti-connexin 43 and anti-connexin 26 agent(s).

The agents of the invention of the may be administered to a subject in need of treatment, such as a subject with any of the diseases or conditions mentioned herein. The condition of the subject can thus be improved. The anti-connexin agents may thus be used in the treatment of the subject's body by therapy. They may be used in the manufacture of a sustained release delivery device to treat any of the conditions mentioned herein. Thus, in accordance with the invention, there are provided formulations by which cell-cell communication, and/or cell-extracellular environment, can be downregulated in a transient and site-specific manner.

The anti-connexin and other agents may be present in a substantially isolated form. It will be understood that the sustained release delivery devices may be prepared with carriers or diluents that will not interfere with the intended purpose of the product and still be regarded as substantially isolated. A preparation of the invention for use in the manufacture of sustained release delivery devices may also be in a substantially purified form, in which case it will generally comprise about 80%, 85%, or 90%, e.g., at least about 95%, at least about 98% or at least about 99% of the polynucleotide (or other anti-connexin agent) or dry mass of the preparation.

The sustained release delivery devices of the invention may, for example, be prepared with solutions, suspensions, instillations, salves, creams, gels, foams, ointments, emulsions, lotions, paints, sustained release formulations, or powders, and typically contain about 0.1%-95% of active ingredient(s), preferably about 0.2%-70%. In certain embodiments, formulations for use in the manufacture of sustained release delivery devices include slow or delayed release preparations.

Foam and gel preparations may be formulated to be delivered from a wound dressing or bandage. Suitable excipients for the formulation of the foam or gel or other base are known in the art and include, but are not limited to, propylene glycol, emulsifying wax, cetyl alcohol, and glyceryl stearate. Potential preservatives that may be included in gels, jellies, or foams include methylparaben and propylparaben.

The term “pharmaceutically acceptable carrier” refers to any pharmaceutical carrier for agents of the invention that does not itself induce the production of antibodies harmful to the individual receiving the sustained release delivery device, and which can be administered without undue toxicity.

Pharmaceutically acceptable salts can also be present in the sustained release delivery devices of the invention, e.g., mineral acid salts such as hydrochlorides, hydrobromides, phosphates, sulfates, and the like; and the salts of organic acids such as acetates, propionates, malonates, benzoates, and the like.

Suitable carrier materials for use in sustained release delivery devices of the invention include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Where the anti-connexin agent is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides, for example. The the sustained release delivery devices of the invention may contain such transfection agents. Examples of these agents include cationic agents (for example calcium phosphate and DEAE-dextran) and lipofectants (for example Lipofectam™ and Transfectam™), and surfactants.

Where the anti-connexin agent comprises a polynucleotide, conveniently, the the sustained release delivery devices of the invention may include a surfactant to assist with polynucleotide cell penetration or the formulation may contain any suitable loading agent. Any suitable non-toxic surfactant may be included, such as DMSO. Alternatively, a transdermal penetration agent such as urea may be included.

Also featured are sustained release drug delivery devices suitable for sustained administration of an anti-connexin agent, comprising a scaffold core and at least one coating. The at least one coating may be selected from (a) a copolymer coating comprising an anti-connexin agent and a biodegradable and biocompatible copolymer and (b) a coating comprising a biodegradable polyester and an anti-connexin modulating agent. In some aspects, the sustained release drug delivery device may comprise two or more coatings comprising an anti-connexin agent and a biodegradable and biocompatible copolymer and/or two or more coatings comprising a biodegradable polyester and an anti-connexin agent.

Each coating may comprise one or more layers. In some embodiments, each coating may comprise one to ten layers, for example 2, 3, 4, 5, 6, 7, 8, 9, or 10 layers.

In some embodiments, the sustained release drug delivery device may comprise at least one copolymer coating comprising an anti-connexin agent and a biodegradable and biocompatible copolymer and at least one coating comprising a biodegradable polyester and an anti-connexin modulating agent, for example. In some aspects, the sustained release drug delivery device may have an inner coating comprising a biodegradable polyester and an anti-connexin modulating agent and an outer coating comprising an anti-connexin agent and a biodegradable and biocompatible copolymer. In this embodiment, both the inner coating and outer coating may comprise more than one layer, for example from one to ten layers. In some embodiments both the inner and outer coating may comprise four layers.

In some embodiments, the scaffold core may comprise any biodegradable material. In some embodiments, the scaffold core may comprise a biodegradable, porous, flexible material. For example, in some embodiments, the scaffold core may comprise a connective tissue blended with a biodegradable polymer. In some aspects, the connective tissue may comprise one or more of the following connective tissues: collagen, elastin, and chondroitin-4-sulfate. In some aspects, the connective tissue may be present at an amount about 50-99% collagen (w/w). In some aspects, the biodegradable polymer may comprise biodegradable polyester polymer. In some aspects, the polyester polymer may comprise one or more of the following selected biodegradable polyester polymers: poly(L-lactide), poly(glycolide), poly(DL-lactide), poly(dioxanone), poly(DL-lactide-co-L-lactide), poly(DL-lactide-co-glycolide), poly(glycolide-co-trimethylene carbonate), and poly(caprolactone) (“polycaprolactone”). In some aspects, the polyester polymer may comprise polycaprolactone (PCL). In some aspects, the biodegradable polyester polymer may be pretreated by methods known in the art so as to enhance the rate of hydrolysis. In some aspects, the amount of polycaprolactone may be present at an amount about 1-50% polycaprolactone (w/w). In some aspects, the molecular weight of the polycaprolactone may range from 5,000 Da to 5,000,000 Da. In some aspects, the molecular weight of the polycaprolactone may range from 10,000 to 1,000,000 Da. In some aspects, the molecular weight of the polycaprolactone may range from about 50,000 to about 500,000 Da. In some aspects, the molecular weight of the polycaprolactone may range from 70,000 to 140,000 Da. In some aspects, the molecular weight of the polycaprolactone may range from about 75,000 to 100,000 Da. Practitioners in the art will appreciate that there are several methods of reporting a polymer molecular weight, and that the molecular weight value reported herein is the Mₙ (number average molecular weight).

In other embodiments, the scaffold core may comprise a non-biodegradable substrate. In some aspects, the non-biodegradable substrate may be a nylon surface. In some aspects, the nylon surface may be part of a bandage or other wound-dressing type of material.

In some aspects, the collagen and the polymer comprising the scaffold may be processed to produce a scaffold. The scaffold may be porous or non-porous. The scaffold may be created by electrospinning the mixture of the collagen and the polymer comprising the scaffold into fibers to create a scaffold sheet, from which the desired scaffold shape may be obtained. In some aspects, the shape obtained from the scaffold sheet may be a disk obtained from the sheet, for example, cut by a laser or other cutting tool, and/or punched out of the sheet using a biopsy punch. Alternatively, by controlling fiber orientation during fiber deposition, three-dimensional scaffolds may be fabricated having a desired shape other than a disk. Alternatively, polymer sheets may be cast by methods known to those in the art, including but not limited to spin-coating, dip-coating, and spray-drying. In addition, three-dimensional printing may be used to obtain a scaffold having any desired shape, and may be custom printed for each subject.

Several forms of simple spun alginate or collagen scaffold, or microspheres, without an anti-connexin agent, were previously shown to induce a foreign body reaction at the wound edge, which can further retard the healing process (Gilmartin, D. et al., Adv. Healthcare Mater. (2013), 2: 1151-1160). As part of the foreign body inflammatory reaction there an elevation of the protein levels of the gap junction connexin 43 in the wound edge keratinocytes and fibroblasts was previously observed with an untreated scaffold, which inhibited the migration of these cells. In the study the scaffolds were soaked in a thermo-reversible medium containing connexion 43 antisense oligodeoxynucleotides transiently alleviated some of the foreign body reaction and encouraged outgrowth of the wound edge keratinocytes. However, the medium associated with the scaffold was shortlived and soon the inflammatory response returned and the scaffold was attacked by leukocytes.

In the present invention, the sustained release delivery devices of the invention are coated with a mixture comprising an anti-connexin agent, preferably anti-connexin 26 and anti-connexin 43 agents. The mixture typically also comprises a polymer which temporarily binds the anti-connexin agent to the scaffold (“binding polymer”, or “eluting polymer”). Such polymers may include or exclude, for example, one or more of the following homopolymers or copolymers of the following selected polymers: poly(lactic-co-glycolic acid (PLGA), poly(L-lactide), poly(glycolide), poly(DL-lactide), poly(dioxanone), poly(DL-lactide-co-L-lactide), poly(DL-lactide-co-glycolide), poly(glycolide-co-trimethylene carbonate), and polycaprolactone (PCL), Multiple coatings may be applied to the scaffold to achieve a desired elution profile of the anti-connexin agent. In some embodiments, each coating layer may be applied by immersing the scaffold in a solution of the dissolved binding polymer and anti-connexin agent. The solution may comprise a mixture of solvents to dissolve or suspend both the polymer and the anti-connexin agent. As a non-limiting example, PCL and PLGA are soluble in dimethylcarbonate. As a non-limiting example, the specific anti-connexin agent of an asODN is soluble in water. As a non-limiting example, a mixture of PCL or PLGA with an asODN may be generated by homogenization of the solution at room temperature to dissolve or suspend both the asODN and the PCL or PLGA. The scaffold may then be separated from the solution and freeze-dried (lyophilized) to remove the solvent or solvents. The scaffold comprising the first binding polymer layer may be subsequently immersed into another anti-connexin agent containing solution comprising the same binding polymer as used in the prior immersion, or a different binding polymer, and the same anti-connexin agent as used in the prior immersion, or a different anti-connexin agent than that used in a prior immersion. The steps of immersion and lyophilization may be repeated up to forty times to create one or more coatings on the scaffold. In some aspects, a stabilizer may be added to one or more of the dip solutions so as the stabilize the anti-connexin agent during dry storage.

In some embodiments, a combination of layers on the scaffold affords a measured release of the anti-connexin agents. In a preferred embodiment, the release profile of the anti connexin agent will be an immediate early burst at the wound, but also a sustained release thereafter. In a preferred embodiment, the inner (first) layers comprise an asODN mixed with PCL. In a preferred embodiment, an outer (second or subsequent) layer comprises an asODN mixed with PLGA. In a preferred embodiment, some coatings may provide a level of protection for the anti connexin agent against serum nucleases.

Any dose or concentration of anti-connexin agent described herein may be used in the the sustained release delivery devices of the invention, e.g., in the scaffold coatings and/or layers.

The effective dosage of each of the anti-connexin agents employed in the methods and compositions of the invention may vary depending on a number of factors including the particular anti-connexin agent or agents employed, the combinational partner, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

Therapeutically effective doses of anti-connexin agents from about 1 to 100, 100-200, 100- or 200-300, 100- or 200- or 300-400, and 100- or 200- or 300- or 400-500 micrograms are appropriate for delivery by the sustained release delivery devices of the invention. Doses from about 1-1000 micrograms are also appropriate. Doses up to 1-3, 3-10, 10-30 and 30-50 milligrams per agent may also be used for sustained release delivery devices of the invention. The compounds described herein may be calculated to be present at amounts ranging from about 100 or 1000 to 50,000 micrograms per one square millimeter to one square centimeter of scaffold. They can also be calculated to be present at amounts ranging from about 1 to about 50 mg of coating substance, for example, from 1 or 3 mg/mL to 3-10 or 10-30 mg/mL, and up to 50 to 100 mg/mL and may be calculated by using the total volume of coating material or by the volume of one or more layers of each coating substance. Doses are adjusted appropriately when the anti-connexin agent or other agents are provided in the form of a bandage or dressing, typically upward to maintain the desired total dose administration.

Alternatively, in the case of anti-connexin oligonucleotides or anti-connexin peptides and peptidomimetics, for example, and other agents, the dosage of each of the gap junction modulation agents in the compositions may be determined by reference to concentration in sustained release delivery devices of the invention relative to the size, length, depth, area or volume of the area to which it will be applied. For example, in certain topical applications, dosing and application of sustained release delivery devices of the invention may be calculated based on mass (e.g., grams) of or the concentration in a pharmaceutical composition (e.g., μg/ul) per length, depth, area, or volume of the area of application. Useful dose applications range from about 1 to about 10 micrograms or milligrams per square centimeter of wound size. Certain doses will be about 1-2, about 1-5, about 2-4, about 5-7, and about 8-10 micrograms or milligrams per square centimeter of wound size. Other useful doses are greater than about 10 micrograms or milligrams per square centimeter of wound size, including at least about 15 micrograms or milligrams per square centimeter of wound size, at least about 20 micrograms or milligrams per square centimeter of wound size, at least about 25 micrograms or milligrams per square centimeter of wound size, about 30 micrograms or milligrams per square centimeter of wound size, at least about 35 micrograms or milligrams per square centimeter of wound size, at least about 40 micrograms or milligrams per square centimeter of wound size, at least about 50 micrograms or milligrams per square centimeter of wound size, and at least about 100 to at least about 150 micrograms or milligrams per square centimeter of wound size. Other doses include about 150-200 micrograms or milligrams per square centimeter, about 200-250 micrograms or milligrams per square centimeter, about 250-300 micrograms or milligrams per square centimeter, about 300-350 micrograms or milligrams per square centimeter, about 350-400 micrograms or milligrams per square centimeter, and about 400-500 or milligrams micrograms per square centimeter.

In certain embodiments, the anti-connexin agent composition may be applied at about 0.01 micromolar (μM) or 0.05 μM to about 200 μM, or up to 300 μM or up to 1000 μM or up to 2000 μM, up to 3000 μM, up to 3200 μM, up to 3000 μM, up to 3500 μM, up to 4000 μM, up to 4500 μM, up to 5000 μM, up to 5500 μM, up to 6000 μM, up to 6500 μM, up to 7000 μM, up to 7500 μM, up to 8000 μM, up to 8500 μM, up to 9000 μM, up to 9500 μM, up to 10000 μM, up to 10500 μM, up to 11000 μM, up to 11500 μM, up to 12000 μM, up to 12500 μM, up to 13000 μM, up to 13500 μM, up to 14000 μM, up to 14500 μM, up to 15000 μM, up to 17500 μM, up to 20000 μM, up to 22500 μM, up to 25000 μM, up to 27500 μM, up to 30000 μM, up to 32500 μM, up to 35000 μM, up to 37500 μM, up to 40000 μM, up to 42500 μM, up to 45000 μM, up to 47500 μM, or up to 50000 μM or more final concentration at the treatment site and/or adjacent to the treatment site, and any doses and dose ranges within these dose numbers. Preferably, the composition comprising the anticonnexin agent, for example, the anti-connexin polynucleotide, peptide, or peptidomimeticis applied at about 0.05 μM to about 100 μM final concentration, more preferably, the anti-connexin agent composition is applied at about 1.0 μM to about 50 μM final concentration, and more preferably, the anti-connexin agent composition is applied at about 5-10 μM to about 30-50 μM final concentration. Additionally, the combined anti-connexin agent composition is applied at about 8 μM to about 20 μM final concentration, and alternatively the anti-connexin agent composition is applied at about 10 μM to about 20 μM final concentration, or at about 10 to about 15 μM final concentration. In certain other embodiments, the anti-connexin agent is applied at about 10 μM final concentration. In yet another embodiment, the anti-connexin agent composition is applied at about 1-15 μM final concentration. In other embodiments, the anti-connexin agent is applied at about a 20 μM, 30 μM, 40 μM, 50 μM, 60 μM, 70 μM, 80 μM, 90 μM, 100 μM, 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM or 2000 μM-3000 μM or greater.

Anti-connexin agent dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. These dose amounts in milligrams, e.g., from 1-50 and up to 500 mg, are also provided, as noted above.

Dose amounts for anti-connexin and other agents may further include from 0.1 to about 50 mg/mL, from 0.5 to about 50 mg/mL, from 1 to about 40 mg/mL, or about 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, 37, 38, 39, 40, 41, 42, 43, 44, 45, 46, 47, 47, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/mL, and any doses and dose ranges within these dose concentrations.

All doses and dose ranges referenced herein are applicable, for example, to anti-connexin oligonucleotides. These dose ranges are also applicable, for example, to anti-connexin peptides anti-connexin mimetic peptides and anti-connexin peptidomimetics, and to anti-cadherin and anti-catenin agents.

In a preferred embodiment, the combined use of two or more agents, e.g., anti-connexin agents has an additive, synergistic or super-additive effect. In some cases, the combination, for example, of one or more anti-connexin polynucleotide and one or more anti-connexin peptides or peptidomimetics, or other anti-connexin or other agents administered in combination with either or both, have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of one or more anti-connexin polynucleotides and one or more anti-connexin peptides or peptidomimetics, or other anti-connexin agents or other agents administered in combination with either or both, is statistically significantly higher than the sum of the wound healing produced by the individual administration of either of the agents alone. Whether produced by combination administration of one or more anti-connexin polynucleotides and one or more anti-connexin peptides or peptidomimetics, or other anti-connexin or other agents administered in combination with either or both, is “statistically significantly higher” than the expected additive value of the individual compounds may be determined by a variety of statistical methods as described herein and/or known by one of ordinary skill in the art. The term “synergistic” refers to a type of supra-additive inhibition in which both the anti-connexin polynucleotide and anti-connexin peptide or peptidomimetic, or other anti-connexin agents administered in combination with either or both, individually have the ability to promote wound healing. The term “potentiated” refers to type of supra-additive effect in which one of the anti-connexin polynucleotide, anti-connexin peptides or peptidomimetics, or other anti-connexin or other agents administered in combination with either or both, individually has the increased ability to promote wound healing. In general, potentiation may be assessed by determining whether the combination treatment produces a mean wound healing increase in a treatment group that is statistically significantly supra-additive when compared to the sum of the mean wound healing increases produced by the individual treatments in their treatment groups respectively. The mean wound healing increase may be calculated as the difference between control group and treatment group mean wound healing using methods known in the art. Whether a synergistic effect results from a combination treatment may also be evaluated by statistical methods known in the art.

Any method known or later developed in the art for analyzing whether a supra-additive effect exists for a combination therapy is contemplated for use in evaluating for anti-connexin and other agents for use in combination.

Preferably one or more anti-connexin polynucleotides and one or more anti-connexin peptides or peptidomimetics, or other anti-connexin agents administered in combination with either or both, are delivered by topical administration (peripherally or directly to a site), including but not limited to topical administration using solid supports (such as dressings and other matrices). In one embodiment, the solid support comprises a biocompatible membrane or insertion into a treatment site. In another embodiment, the solid support comprises a scaffold or matrix, or a dressing or bandage. In one embodiment of the invention, the solid support composition may be a slow release solid support composition, in which the one or more anti-connexin polynucleotides and one or more anti-connexin peptides or peptidomimetics, or other anti-connexin or other agents to be administered in combination with either or both, is dispersed in a slow release solid matrix such as those described herein. Preferably, the solid support composition is sterile or low bio-burden.

The delivery of anti-connexin agent(s), e.g., for downregulation of connexin expression, or blockade or inhibition of connexon opening or activity, therefore will modulate communication between the cells, or loss into the extracellular space in the case of connexon regulation, and minimize cell loss or consequences of injury.

While the delivery period will be dependent upon both the site at which the downregulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-connexin polynucleotides and one or more anti-connexin peptides or peptidomimetics, or other anti-connexin agents in combination with either or both, in a sustained release drug delivery device, particularly in the form of a formulation for continuous or slow-release administration.

As noted, the one or more sustained release drug delivery devices of the invention may be administered before, during, immediately following wounding, for example, or within about 180, about 120, about 90, about 60, or about 30 days, but preferably within about 10, about 9, about 8, about 7, about 6, about 5, about 4, about 3, or about 2 days or less, and most preferably within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute following wounding, for example.

Any of the methods of treating a subject having a wound and/or condition referenced or described herein may utilize the administration of any of the doses, sustained release drug delivery devices, formulations, and/or compositions herein described.

### Dressings and Matrices

In one aspect, agents of the invention, e.g. one more anti-connexin polynucleotides and/or one or more anti-connexin peptides or peptidomimetics are provided in the form of a dressing or matrix. In certain embodiments, the agents of the invention are provided in the form of a liquid, semi-solid or solid composition for application directly, or the composition is applied to the surface of, or incorporated into, a solid contacting layer such as a dressing gauze or matrix. The dressing composition may be provided for example, in the form of a fluid or a gel. One or more anti-connexin polynucleotides and one or more anti-connexin peptides or peptidomimetics may be provided in combination with conventional pharmaceutical excipients for topical application. Suitable carriers include: pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof, and hydrogels containing polyacrylic acid (Carbopols). Suitable carriers also include creams/ointments used for topical pharmaceutical preparations, e.g., creams based on cetomacrogol emulsifying ointment. The above carriers may include or exclude, for example, alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include or exclude, for example, the following with one or more anti-connexin polynucleotides and one or more anti-connexin peptides or peptidomimetics (or other anti-connexin or other agents as described herein to be administered in combination with either or both): absorptive dressings, alginate dressings, antimicrobial dressings, biological dressings, biosynthetic dressings, collagen dressings, composite dressings, contact layer dressings, foam dressings, gauze dressings, woven dressings, hydrocolloid dressings, hydrogel dressings, amorphous hydrogel dressings, impregnated hydrogel dressings, hydrogel sheets, impregnated dressings, filler dressings, liquid dressings, transparent film dressings, silicone gel sheet dressings and elastic bandages. Examples include the above dressings and bandages made to contain, for example, anti-connexin 26 and anti-connexin 43 agents, such as, for example, anti-connexin 26 and anti-connexin 43 polynucleotides, e.g., antisense molecules.

### Combination Wound Treatment

The present invention is directed to sustained release drug delivery devices and their manufacture and use wherein the devices comprise therapeutically effective amounts of one or more anti-connexin polynucleotides and/or one or more anti-connexin peptides or peptidomimetics, or other anti-connexin or other agents in combination with one or more of an anti-connexin polynucleotide and/or an anti-connexin peptide or peptidomimetic or other agent. The compositions are useful in enhancing or promoting healing of wounds, including acute wounds and wounds that do not heal at expected rates, such as chronic wounds and other wounds that may be slow to heal or refractory to conventional wound treatment or wound healing promoting therapies.

Equally, in instances of other tissue damage (particularly wounds) the methods and compositions of the invention are effective in promoting the wound healing process, reducing swelling and inflammation, and in minimizing scar formation. The formulations have clear benefit in the treatment of wounds, whether the result of external trauma (including burns), internal trauma, or surgical intervention, as well as chronic wounds.

Accordingly, in one aspect, the invention provides sustained release drug delivery devices for use in therapeutic treatment, which comprises the anti-connexin and other agents described herein. In one preferred embodiment, the sustained release drug delivery devices comprises an anti-connexin 26 and an anti-connexin 43 agent, for example anti-connexin 26 and an anti-connexin 43 antisense polynucleotides and/or anti-connexin 26 and an anti-connexin 43 hemichannel blockers.

In one preferred form, the sustained release drug delivery device contains one or more antisense polynucleotides to the mRNA of two connexin proteins. In another preferred form, the composition comprises one or more anti-connexin peptides or peptidomimetics, or a gap junction or hemichannel phosphorylation agent or connexin carboxy-terminal polypeptide. Most preferably, these connexins are connexin 26 and connexin 43.

In another form, the sustained release drug delivery device comprises an anti-connexin peptide or peptidomimetic and an antisense polynucleotide to the mRNA of two connexin proteins. Most preferably, these connexins are connexin 26 and connexin 43.

The sustained release drug delivery devices may comprise polynucleotides or anti-connexin peptides, or other anti-connexin agents with either or both, that are directed to more at least two connexin proteins and/or a catenin or cadherin. Preferably, two of the connexin proteins to which polynucleotides or anti-connexin peptides or other anti-connexin agents are directed are connexins 26 and 43. Other connexins to which the polynucleotides or anti-connexin peptides or other anti-connexin agents are directed may include or exclude, for example, connexins 30, 30.3, 31.1, 32, 36, 37, 40, 40.1, 44.6, 45, and 46. Suitable exemplary polynucleotides (and ODNs) directed to various connexins are set forth in Table 1. Suitable anti-connexin peptides are also provided herein. Suitable gap junction or hemichannel phosphorylation agents and connexin carboxy-terminal polypeptides are known in the art, e.g., ACT-1.

### Kits, Medicaments and Articles of Manufacture

One or more anti-connexin polynucleotides and one or more anti-connexin peptides or peptidomimetics and/or other anti-connexin or other agents, such as a gap junction or hemichannel phosphorylation agent or connexin carboxy-terminal polypeptide, may also be used in the manufacture of the sustained release drug delivery device.

In one aspect, the invention provides a kit comprising one or more sustained release drug delivery devices described herein. For example, the kit may include a sustained release drug delivery device comprising an effective amount of one or more anti-connexin polynucleotides and one or more anti-connexin peptides or peptidomimetics and/or other anti-connexin agents, such as a gap junction or hemichannel phosphorylation agent or connexin carboxy-terminal polypeptide, or other agent(s) as described herein.

Sustained release drug delivery devices as articles of manufacture are also provided, containing a sustained release drug delivery device of the invention as described herein and instructions for use for the treatment of a subject. For example, in another aspect, the invention includes an article of manufacture comprising a sustained release drug delivery device having a therapeutically effective amount of one or more anti-connexin polynucleotides and one or more anti-connexin peptides or peptidomimetics and/or other anti-connexin agents, such as a gap junction or hemichannel phosphorylation agent or connexin carboxy-terminal polypeptide, and/or other agents, and instructions for use, including use for the treatment of a subject.

### Treatment

The sustained release drug delivery device of the invention may be used in conjunction or combination with a composition for promoting the healing of wounds, and can also reduce swelling, inflammation and/or scarring. The compositions and formulations of the invention may also be used in conjunction or combination with a composition for promoting and/or improving the healing of acute or chronic wounds.

In one aspect the sustained release drug delivery device of the invention is directed to a method of promoting or improving wound healing in a subject, comprising sustained or slow and/or sequenced administration of therapeutically effective amount of one or more anti-connexin polynucleotides and/or one or more anti-connexin peptides or peptidomimetics or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, or other agents as described herein. In certain embodiments, the administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, is effective to reduce inflammation, promote cell migration to accelerate wound closure and healing, and/or to facilitate epithelial growth and surface recovery. In certain embodiments, the administration of one or more anti-connexin polynucleotides and one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, is effective to reduce or prevent scar formation.

In yet a further aspect, the invention provides a method of decreasing scar formation and/or improving scar appearance in a patient who has suffered a wound, e.g., a surgical wound (such as in, for example, cosmetic and other surgeries), which comprises the step of administering a sustained release drug delivery device of the invention comprising one or more anti-connexin polynucleotides and/or one or more anti-connexin peptides or peptidomimetics, or, optionally, one or more anti-connexin polynucleotides and/or one or more anti-connexin peptides or peptidomimetics other anti-connexin agents, such as a gap junction or hemichannel phosphorylation agent or connexin carboxy-terminal polypeptide, or other agents as described herein to said wound to modulate or downregulate expression of one or more hemichannels or connexin or other protein(s), respectively, at and immediately adjacent the site of said wound. Again, the wound may be the result of trauma or surgery, for example, with the formulation being applied to the wound immediately prior to surgical repair and/or closure thereof. As noted herein, in methods to reduce or improve scar formation or appearance, the anti-connexin peptide or peptidomimetic in one embodiment is preferably administered in combination with administration of a suitable amount anti-connexin polynucleotide.

In one aspect the invention is directed to a method of reducing, preventing or ameliorating tissue damage in a subject, comprising administration of a sustained release drug delivery device of the invention.

In a further aspect, the invention is directed to a method of reducing swelling and/or inflammation as part of treating an acute or chronic wound and/or tissue subjected to physical trauma which comprises the step of administering a sustained release drug delivery device of the invention. In one embodiment the wound is the result of physical trauma to tissue, including neuronal tissue such as, for example, the spinal cord.

In one aspect the invention is directed to device suitable for sustained administration of one or more anti-connexin polynucleotides and/or one or more anti-connexin peptides or peptidomimetics, or, optionally, to sustained administration of one or more anti-connexin polynucleotides and/or one or more anti-connexin peptides or peptidomimetics other anti-connexin agents, such as a gap junction or hemichannel phosphorylation agent or connexin carboxy-terminal polypeptide, or other agents as described herein. In one embodiment, the anti-connexin agents are administered via a sustained release drug delivery device of the invention and connexin expression is downregulated over a sustained period of time. In another embodiment, connexin hemichannels are blocked or closed using a sustained release drug delivery device of the invention, in whole or in part, over a preferred period of time. Preferably connexin 26 and 43 expression are downregulated and/or connexin 26 and 43 hemichannel opening is blocked or inhibited, in whole or in part, for a sustained period of time. Conveniently, connexin 26 and 43 expression is downregulated or hemichannels blocked or inhibited for at least about 1, 2, 4, 6, 8, 10, 12, or 24 hours or more. According to one embodiment, the wound is a chronic wound. Suitable subjects include a diabetic subject. Other subjects include, for example, those with peripheral edema, vasculitis, or vascular or cardiovascular disease.

In one aspect, the present invention provides a method of treating a subject having a wound which comprises administration of a sustained release drug delivery device of the invention to the wound. In a further aspect, the present invention provides a method of promoting or improving wound healing in a subject which comprises a sustained release drug delivery device of the invention to a wound. In a further aspect, the present invention provides a method of reducing, preventing or ameliorating swelling and/or inflammation in a subject which comprises administration of a sustained release drug delivery device of the invention to a wound. In a further aspect, the present invention provides a method of reducing, preventing or ameliorating scar formation in a subject which comprises administration of a sustained release drug delivery device of the invention to a wound.

According to another further aspect, the present invention provides a method of promoting or improving wound healing in a subject having a wound which comprises administration or serial administration of a sustained release drug delivery device of the invention to a wound area in an amount effective to reduce inflammation and increase re-epithelialization rates in the wound area. According to one embodiment, the wound is a chronic wound. Subjects that may be treated include diabetic subjects, for example.

In another aspect, methods for treating a subject having a chronic wound are provided. Such methods include administering to the subject a sustained release drug delivery device of the invention comprising an anti-connexin agents capable of inhibiting the expression, formation, or activity of a connexin, or a connexin hemichannel, in combination with another anti-connexin agent.

In one aspect the invention is directed to a method for treatment or prophylaxis of a chronic wound comprising administering to a subject in need thereof a sustained release drug delivery device of the invention. In another embodiment, the chronic wound is a chronic skin wound and a composition of the present invention is administered to the skin or a tissue associated with the skin of said subject for an effective period of time, or multiple applications of a sustained release drug delivery device of the invention over time. A chronic skin wound suitable for treatment may, for example, be selected from the group consisting of pressure ulcers, diabetic ulcers, venous ulcers, arterial ulcers, vasculitic ulcers, and mixed ulcers, and other noted herein. The chronic wound may be an arterial ulcer that comprises ulcerations resulting from complete or partial arterial blockage. The chronic wound may be a venous stasis ulcer that comprises ulcerations resulting from a malfunction of the venous valve and the associated vascular disease. The chronic wound may be a trauma-induced ulcer. The chronic wound may be a persistent epithelial defect.

The following examples which will be understood to be provided by way of illustration only and not to constitute a limitation on the scope of the invention.

### EXAMPLES

### Example 1

### Anti-Cx26 Polynucleotide Design

### DNAzyme Walking

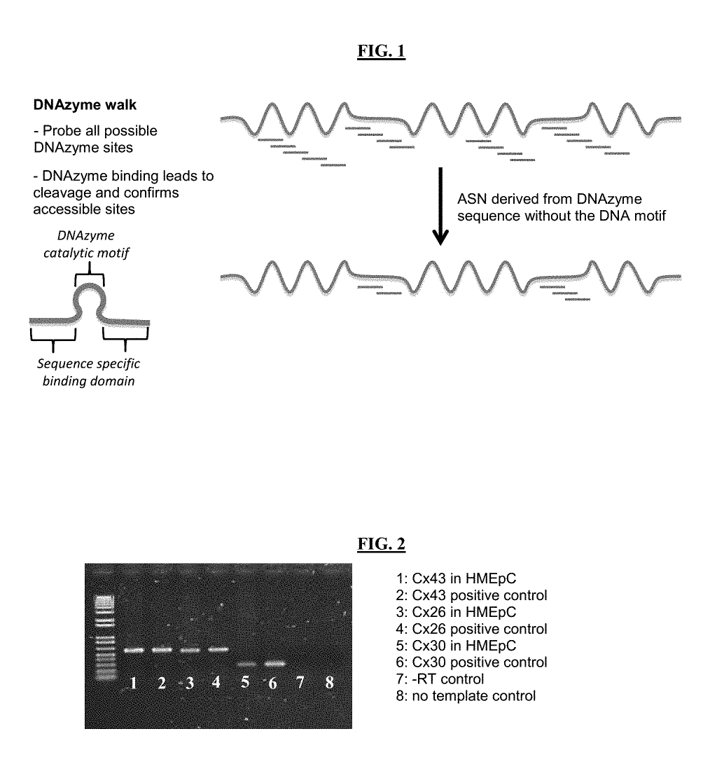

RNAs are single stranded under normal physiological conditions. When a RNA/DNA heteroduplex occurs in a mammalian cell, for example, the RNAse H enzyme is activated to cleave the heteroduplex, leading to degradation of the RNA portion. However, not all parts of an RNA are accessible by antisense (ASN) molecules due to their secondary and tertiary structure. To overcome this hurdle, a DNAzyme walk approach was devised to map out sites that are accessible on the target gene. See, e.g., Law et al., 2006. Briefly, a DNAzyme is a short, single-stranded DNA with two arms that each bind to the target gene in a sequence specific fashion. The two arms bound a central conserved catalytic DNA motif that facilitates cleavage at a purine-pyrimidine junction (i.e., between AG, AT, GC, GT) of the RNA sequence (Cairns, et al., 1999). Cleavage occurs when a DNAzyme successfully binds to the target gene. See FIG. 1.

Here, the ‘walk’ approach entailed designing a library of DNAzymes that covers all possible sites (purine-pyrimidine junction) in the human Cx26 mRNA and determining their activity on the gene. After the DNAzyme assay, fragment sizes were analysed by agarose gel electrophoresis. Lower gel bands and sometimes with the faded original band denoted cleavage by DNAzyme. The intensity of the bands was graded to give a ranking of the degree of accessibility to each site identified.

### Human and Rat Cx26 Clones

Full length human and rat connexin 26 gene sequences were obtained from the NCBI database (www.ncbi.nlm.nih.gov/nucleotide) (see Table 9, below). A T7 promoter sequence was added to the 5′ end of the human and rat genes for RNA transcription in later experiments. Gene synthesis was commercially outsourced. The human and rat connexin 26 genes were cloned in bacterial cells, and clones were then grown and purified and the full length human and rat sequences were confirmed by DNA sequencing.

TABLE 9
Gene sequences derived from NCBI database.

| Gene | Sequence source | Sequence ID |
|---|---|---|
| Human Cx26 | GenBank accession # | SEQ. ID. NO: 149 |
| | NM_004004.5 | |
| Rat Cx26 | GenBank accession # | SEQ. ID. NO: 150 |
| | NM_001004099.1 | |

### Human and Rat Cx26 DNAzyme Screening

A summary of DNAzyme screening results of human and rat Cx26 mRNA is shown in Table 10 below. 460 DNAzyme sites (purine/pyrimidine junction) covering part of the 5′ untranslated region (UTR) along with the full coding region and 3′-UTR of human Cx26 were tested on human Cx26 mRNA (“DNAzyme walk”). DNAzymes were synthesized using standard techniques. The results of the DNAzyme testing are summarized in Appendix A, at the end of this disclosure, and the details of the DNAzyme testing are shown in Appendices A and B, at the end of this disclosure.

TABLE 10
Summary of DNAzyme screening results
of human and rat Cx26 mRNA

| | Human Cx26 mRNA | | Rat Cx26 mRNA | |
|---|---|---|---|---|
| | | Positive Dz | | Positive Dz |
| Human Dz | Dz tested | cut sites | Dz tested | cut sites |
| Dz walk (5′-UTR | 173 | 87 | 87 | 10 |
| and coding region) | | | | |
| Dz walk (3′ utr) | 287 | 146 | — | — |
| Total | 460 | 233 | 87 | 10 |

In Appendices A and B, at the end of this disclosure, DNAzyme (Dz) activity was allocated with ratings, as follows: ‘−’: no cleavage; ‘(+)’: weak cleavage; ‘+’: some cleavage; ‘++’: good cleavage; ‘+++’ or more: very good cleavage. If shown as positive (see grey boxes), then that Dz site was considered accessible and therefore is expected to give activity if included as part of antisense molecule that incorporate the hybridizing nucleotides in the corresponding Dz. All DNAzymes with positive activity on the human Cx26 mRNA were then tested on rat Cx26 mRNA. Black boxes in Appendix A indicate sequences that were not tested on rat Cx26 mRNA.

As shown above, a total of 233 accessible sites were identified in human Cx26 mRNA. Of these, 20 sites in the coding region and 17 sites in the 3′-UTR were assessed as being highly accessible. Sequence homology between a DNAzyme and its target sequence was found to be an important factor for binding and consequently DNAzyme activity. This is supported by the poor activity of many of the high activity human DNAzymes on rat Cx26 mRNA.

### Human and Rat Cx26 DNAzyme Crossreactivity

To test species cross-reactivity, DNAzymes in the human Cx26 coding region that showed cleavage (including those with very poor cutting) were tested on rat Cx26 mRNA. As shown in Table 12, below, most of the human DNAzymes contained mismatches against the rat Cx26 mRNA sequence, and their accessibility to the rat Cx26 mRNA was also poor.

TABLE 12
Summary of human Cx26 coding region
DNAzymes activity in rat Cx26 mRNA

| | Human Cx26 mRNA | Rat Cx26 mRNA |
|---|---|---|
| Human-Rat | +ve Dz cut sites | +ve Dz cut sites |
| Dz - 100% homology | 1 | 1 |
| Dz - mismatch | 86 | 9 |
| Total | 87 | 10 |

Only 10 out of 87 DNAzymes active against human Cx26 mRNA showed some activity against rat Cx26 mRNA. One DNAzyme with 100% homology against both human and rat Cx26 mRNA showed activity in both human and rat Cx26 coding regions; the other DNAzymes tested had varying numbers of mismatches with the rat sequence.

### Example 2

### Selecting Cell Lines to Test Anti-Cx26 Polynucleotide-Mediated Knockdown

This example describes the in vitro cell culture testing of antisense (ASN) knockdown activity of various anti-connexin polynucleotides. In the experiments described herein, mammalian cell lines were grown and tested for connexin 26, 30, and 43 expression at the gene and protein levels by RT-PCR and immunocytochemistry, respectively. Details of PCR primers and antibodies are listed in Table 13, below.

TABLE 13
Primers and antibodies used for detecting connexin expression

| | Primer-Forward | Primer-Reverse | Antibody |
|---|---|---|---|
| Cx43 | ATGAGCAGTCTGCCTTTCGT | GGGTCGCTCTTTCCCTTAAC | Sigma, Cat. # |
| | (SEQ. ID. NO: 151) | (SEQ. ID. NO: 152) | C6219 |
| Cx26 | ACTCCACCAGCATTGGAAAG | GACATTCAGCAGGATGCAAA | Des; Novus, |
| | (SEQ. ID. NO: 153) | (SEQ. ID. NO: 154) | Cat. # NB100-57840 |
| Cx30 | TGCAAACGGGGCTGACCTCC | TGGGGACGAGCTTTATTGAG | Invitrogen, |
| | (SEQ. ID. NO: 155) | (SEQ. ID. NO: 156) | Cat. # 71-2200 |

Three human cell lines were tested for Cx43, Cx26, and Cx30 expression. Results of these experiments are summarized in Table 14, below.

TABLE 14
Connexin expression in various mammalian cell lines

| Cell Line Information | | | | | | | | | | |
|---|---|---|---|---|---|---|---|---|---|---|
| | | | | Literature | Connexin Expression Profile | | | | | |
| Cell | | | | on | Gene | | | Protein | | |
| Line | Source | Origin | Type | Connexin | Cx43 | Cx26 | Cx30 | Cx43 | Cx26 | Cx30 |
| HMEpC | 830-05a | Human | Primary | NA | ✓ | ✓ | ✓ | ✓ | ✓ | x |
| | Cell | mammary | | | | | scarce | | | |
| | Application | epithelial | | | | | | | | |
| | | cells | | | | | | | | |
| HaCaT | T0020001, | Human | Spontaneously | NA | ✓ | ✓ | ✓ | ND | ND | ND |
| | AddexBio | keratinocyte | transformed | | | | | | | |
| HepG2 | HB-8065, | Human | Carcinoma | NA | ✓ | ✓ | x | ND | ND | ND |
| | ATCC | liver | cells | | scarce | scarce | | | | |
| | | epithelial | | | | | | | | |
| | | cells | | | | | | | | |

✓: expressed;
x: not expressed;
ND: Not done.
For HaCaT, the protein profile was not explored because to save money and time only the RNA was ordered for testing so no cells or protein available.
For HepG2 cells, the protein profile was not explored because they have shown poor expression at the transcript levels.

### Cx Expression in HMEpC (Human Mammary Epithelial Cells)

The expression of Cx23, Cx30, and Cx43 was tested by using RTPCR on RNA isolated from HMEpC (human mammary epithelial cells). The forward and reverse primers used were those listed in Table 13, above. In these experiments, Cx23, Cx30, and Cx43 mRNA expression in human hair follicle cells served as positive controls. The results of these experiments are shown in FIG. 2.

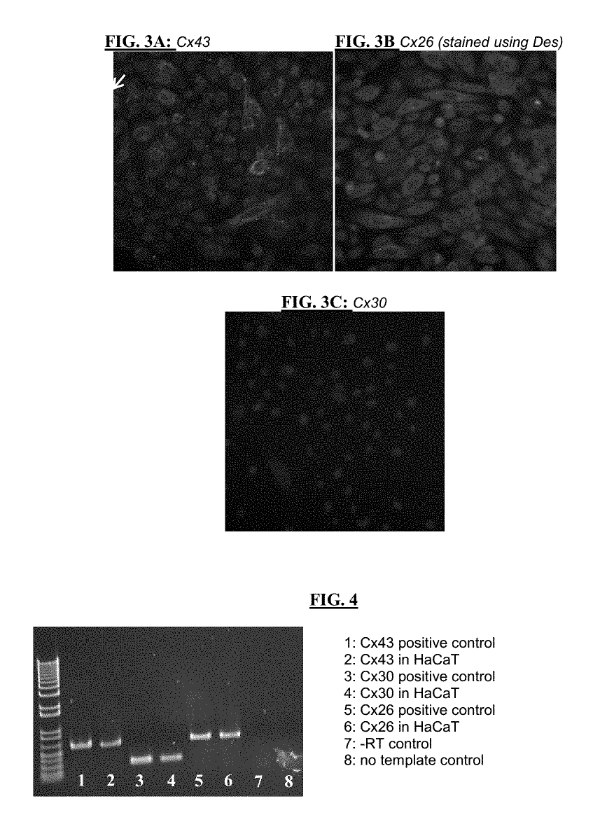

Cx23, Cx30, and Cx43 expression at the protein level was also assessed in HMEpC. The antibodies used for these immunocytochemical analyzes are listed in Table 13, above, and the results of these experiments are shown in FIGS. 3A, 3B and 3C. All images were captured using the same parameters. Cx43 and Cx26 protein were localized intracellular as well as between cell junctions (white arrow). Cell nuclei were stained with DAPI, and in the images shown in FIGS. 3A, 3B and 3C, green represents antibody-stained Cx43 protein.

Expression of each of Cx23, Cx30, and Cx43 was detected by PCR and immunocytochemistry in HMEpC, although Cx30 expression was much lower than the expression levels observed for Cx26 and Cx43 at the gene and protein levels.

### Expression in HaCaT (Human Keratinocytes)

The expression of Cx23, Cx30, and Cx43 was also tested by using RTPCR on RNA isolated from HaCaT (human keratinocytes). The forward and reverse primers used were those listed in Table 13, above. In these experiments, Cx23, Cx30, and Cx43 mRNA expression in human hair follicle cells again served as positive controls. The results of these experiments are shown in FIG. 4, which reveals good expression of all three connexin genes tested.

### Cxn Expression in HeG2 (Human Liver Epithelial Cells)

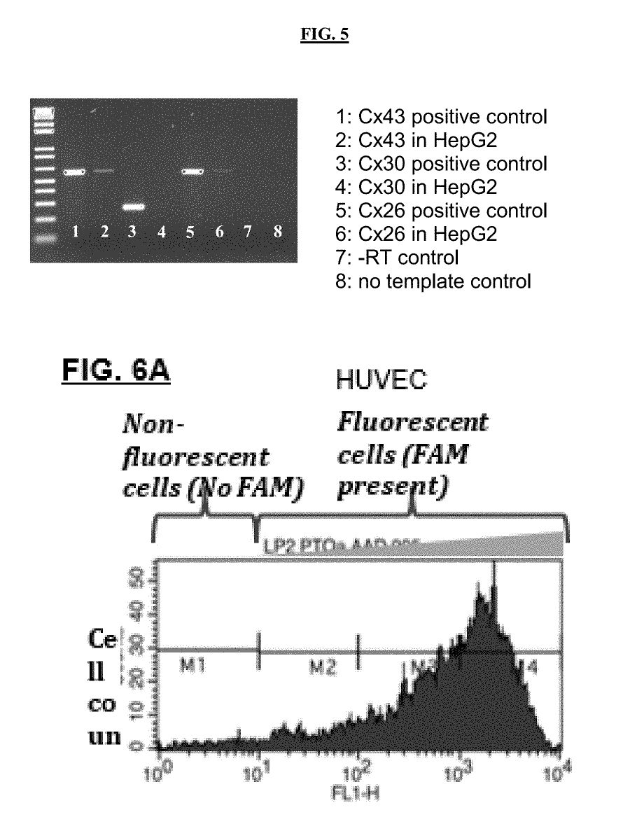

The expression of Cx23, Cx30, and Cx43 was also tested by using RTPCR on RNA isolated from HepG2 (human liver epithelial cells). The forward and reverse primers used were those listed in Table 13, above. In these experiments, Cx23, Cx30, and Cx43 mRNA expression in human hair follicle cells again served as positive controls. The results of these experiments are shown in FIG. 5. To summarize, low levels of Cx26 and Cx43 gene expression was detected by RT-PCR; however, no Cx30 expression was detected.

### Conclusions

As a result of these experiments, HMEpC cells were selected for use in further testing of anti-Cx26 effects because HMEpC cells expressed both Cx43 and Cx26 mRNA and protein while only low levels of Cx30 mRNA and minimal levels of Cx30 protein were detected. Given the very low levels of Cx30 detected at the gene and protein levels, less cross-reactivity with anti-Cx26 polynucleotides was anticipated. Also, these results show that HaCaT cells will likely be useful for testing ASN combination therapies or anti-Cx polynucleotides that target multiple connexin species since solid Cx26, Cx30, and Cx43 expression was detected at the gene level.

### Example 3

### Anti-Cx26 Polynucleotide-Mediated Knockdown In Vitro

This example describes the in vitro cell culture testing of antisense (ASN) knockdown of anti-Cx26 polynucleotides in HMEpC cells. Transfection efficiency in HMEpC was first examined by FACS (fluorescence-activated cell sorting) to quantify the amount of fluorescent-tagged ASN (200 nM FAM-tagged Cx43 ASN: LP2-PTO; see Table 15 below) in cells at 4 hr post-transfection.

TABLE 15
ASN polynucleotide for FACS analysis (“nt” = nucleotide)

| | Fluorescent- | | Length | |
|---|---|---|---|---|
| SEQ. ID. | tagged ASN | Sequence | (nt) | Backbone |
| SEQ. ID. NO: 157 | LP2-PTOFAM | CCAGGCTGACTCAACCGCTG | 20 | PTO |
| | (037501) | | | |

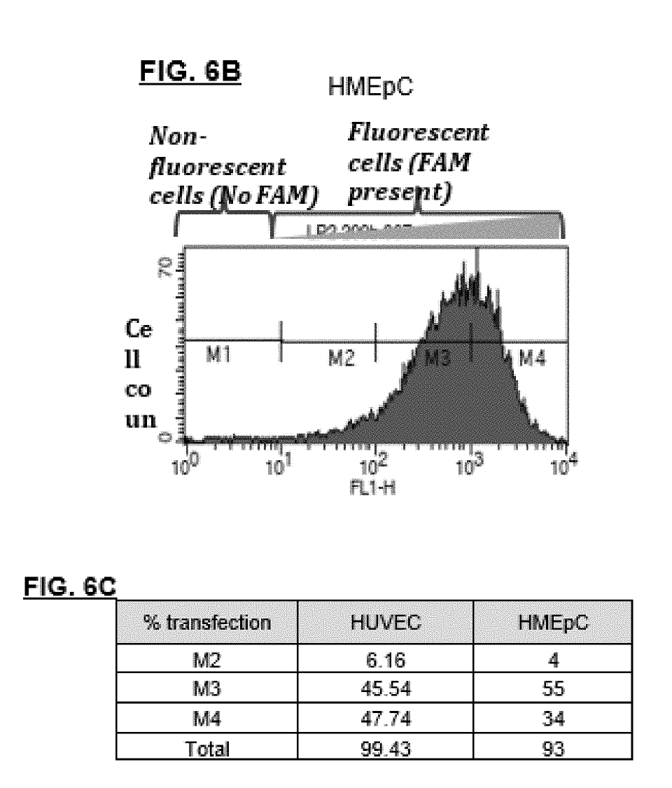

Prior to the transfection, HMEpC were first grown in 6-wells plate for 24 hr. Cells were seeded so that by the time of transfection, confluency was around 60-75%. The LP2-PTO FAM-tagged ASN was transfected using the cationic lipid based transfection reagent Oligofectamine (Invitrogen). Transfection efficiency of this ASN in HMEpC was 93%, with the majority of cells accumulated in the M3/M4 region where the fluorescence level was higher than in the M2 region, indicating that not only most cells had taken up ASN but each cell contained multiple copies of the ASN (see FIGS. 6A, 6B and 6C). Human umbilical vein endothelial cells (HUVECs) were shown to have a slightly greater transfection efficiency of 99.43% with the LP2-PTO FAM-tagged ASN. In addition, the percentage of HUVECs in M3 was similar to M4, whereas the percentage of HMEpCs in M3 was greater than M4, indicating that the amount of the ASN accumulated per cell was also higher in HUVEC.

FIGS. 6A, 6B and 6C show the results of these transfection experiments in HUVECs and HMEpCs. FIGS. 6A and 6B are representative micrographs of FACS results in HUVEC and HMEpCs, respectively. Increasing levels of fluorescence indicated the transfection efficiency of the LP2-PTO FAM-tagged ASN. Both cell types showed considerable accumulation of FAM-tagged LP2-PTO by 4 hr., and hence the fluorescence levels shifted to the M3/M4 regions. FIG. 6C summarizes the FACS results, showing the percentage of cells in each fluorescent region.

### Example 4

### Anti-Cx26 Polynucleotide Screening in HMEPC

This example describes the screening of selected anti-Cx26 polynucleotides in HMEpC cells.

### Introduction

Based on the results of the DNAzyme screening of Cx26 mRNA, there were 20 sites within the RNA's coding sequence that were judged to be highly accessible. Sequence alignments using BLAST indicated that seven out of these 20 ASNs showed low predicted cross-reactivity with off-target genes (15 out of 20 bp) (see Table 16, below); they were therefore selected for in vitro screening in HMEpC cells.

TABLE 16
Anti-Cx26 ASNs targeting highly accessible coding region targets.

| | | | sense seq (rc)-Target on | Dz rating |
|---|---|---|---|---|
| ASN Code | ASN sequence | ASN site | human Cx26 mRNA | (hCx26 mRNA) |
| H26_3501 | TCTCCCCACACCTCCTTTGC | 342-361 | GCAAAGGAGGTGTGGGGAGA | +++ |
| 0 | (SEQ. ID. NO: 158) | | (SEQ. ID. NO: 159) | |
| H26_5500 | CTTCTTCTCATGTCTCCGGT | 505-524 | ACCGGAGACATGAGAAGAAG | ++/+++ |
| 4 | (SEQ. ID. NO: 160) | | (SEQ. ID. NO: 161) | |
| H26_5500 | TCACTCTTTATCTCCCCCTT | 537-556 | AAGGGGGAGATAAAGAGTGA | +++ |
| 7 | (SEQ. ID. NO: 162) | | (SEQ. ID. NO: 163) | |
| H26_6000 | TGGGTTTTGATCTCCTCGAT | 567-586 | ATCGAGGAGATCAAAACCCA | +++ |
| 5 | (SEQ. ID. NO: 164) | | (SEQ. ID. NO: 165) | |
| H26_6000 | TCGATGCGGACCTTCTGGGT | 582-601 | ACCCAGAAGGTCCGCATCGA | ++++ |
| 7 | (SEQ. ID. NO: 166) | | (SEQ. ID. NO: 167) | |
| H26_9000 | CCCAGAACAATATCTAATTA | 856-875 | TAATTAGATATTGTTCTGGG | +++ |
| 4 | (SEQ. ID. NO: 168) | | (SEQ. ID. NO: 169) | |
| H26_9000 | GCTTTTTTGACTTCCCAGAA | 869-888 | TTCTGGGAAGTCAAAAAAGC | ++++ |
| 5 | (SEQ. ID. NO: 170) | | (SEQ. ID. NO: 171) | |

### Methods

HMEpC cells were seeded at 0.8×10⁵ cells per well in 6-wells plate and transfected with ASN-PTO (200 nM) using Oligofectamine®. qPCR assays were used to quantify human Cx26 RNA. Primer sets for reference and target genes were designed and confirmed by size with standard PCR. The primer sets used for the PCR reactions are provided in Table 17, below.

TABLE 17
Primer Sets

| | | | Target Site |
|---|---|---|---|
| Primers | Forward | Reverse | from 5′ end |
| H26 | CGGGAGACAGGTGTTGCG | CTCCTAGGCGGGTCCTGG | 26-102 |
| | (SEQ. ID. NO: 172) | (SEQ. ID. NO: 173) | |
| YWHAZ | ACTTTTGGTACATTGTGGCTTCAA | CCGCCAGGACAAACCAGTAT | Reference |
| | (SEQ. ID. NO: 174) | (SEQ. ID. NO: 175) | gene |

One screening was performed with samples collected at 4 hr. post-transfection to determine the effect of human ASN on Cx26 mRNA levels, as determined by qPCR. Duplicate wells for each anti-Cx26 ASN were used, with 3× triplicates of PCR runs on each well.

FIG. 7 shows the results of these experiments presented as a percentage of vehicle, which also indicates the percentage of knockdown. A range of activity was detected, with the most and least effective human ASN showing 54.74% and 29.67% knockdown of human Cx26 RNA, respectively, 4 hr. post-transfection. H26-60005 was the most effective anti-Cx26 ASN polynucleotide among those tested. This ASN targets nucleotides 567-586 of the human Cx26 mRNA.

### Example 5

### Anti-Cx26 Polynucleotides for Screening in Rat Cells

This example describes the screening of selected anti-Cx26 polynucleotides identified from testing in HMEpC cells in rat cells. Based on cross species screening, 10 human DNAzymes in the coding sequence of the human Cx25 mRNA were shown activity against both human and rat Cx26 mRNA. See Table 18, below.

TABLE 18
Human ASNs derived from DNAzymes that have shown
activity in both human and rat Cx26 mRNA

| | | | | | | Dz | Dz |
|---|---|---|---|---|---|---|---|
| | | | Dz cut | sense seq (rc)- | | rating | rating |
| ASN | ASN sequence- | SEQ | site from | Target on human | SEQ | (human | (rat |
| Code | Human | ID NO. | 5′ end | Cx26 mRNA | ID NO. | Cx26) | Cx26) |
| H26_25 | CGTGCCCCAATCC | SEQ ID | 220 | AGAAGATGGATTGGG | SEQ ID | ++ | (+)/− |
| 003 | ATCTTCT | NO: 176 | | GCACG | NO: 177 | | |
| H26_35 | CATCTCCCCACAC | SEQ ID | 344 | AAAGGAGGTGTGGGG | SEQ ID | ++ | (+) |
| 011 | CTCCTTT | NO: 178 | | AGATG | NO: 179 | | |
| H26_45 | TCGTAGCACACGT | SEQ ID | 402 | TGCAAGAACGTGTGC | SEQ ID | +++ | (+) |
| 001 | TCTTGCA | NO: 180 | | TACGA | NO: 181 | | |
| H26_45 | ATGTGGGAGATGG | SEQ ID | 426 | TACTTCCCCATCTCC | SEQ ID | +/++ | + |
| 008 | GGAAGTA | NO: 182 | | CACAT | NO: 183 | | |
| H26_50 | CGGTAGGCCACGT | SEQ ID | 498 | GCCATGCACGTGGCC | SEQ ID | + | (+) |
| 015 | GCATGGC | NO: 184 | | TACCG | NO: 185 | | |
| H26_50 | ATCTCCTCGATGT | SEQ ID | 567 | TTTAAGGACATCGAG | SEQ ID | ++ | (+) |
| 004 | CCTTAAA | NO: 186 | | GAGAT | NO: 187 | | |
| H26_60 | TCGATGCGGACCT | SEQ ID | 591 | ACCCAGAAGGTCCGC | SEQ ID | ++++ | + |
| 007 | TCTGGGT | NO: 166 | | ATCGA | NO: 167 | | |
| H26_75 | AGTGTTGGGACAA | SEQ ID | 736 | CCTGGCCTTGTCCCA | SEQ ID | +++ | ++ |
| 011 | GGCCAGG | NO: 188 | | ACACT | NO: 189 | | |
| H26_80 | CAATCATGAACAC | SEQ ID | 794 | CTTCACAGTGTTCAT | SEQ ID | (+) | + |
| 011 | TGTGAAG | NO: 190 | | GATTG | NO: 191 | | |
| H26_85 | AGGATGCAAATTC | SEQ ID | 816 | GTGTCTGGAATTTGC | SEQ ID | + | ++ |
| 005 | CAGACAC | NO: 192 | | ATCCT | NO: 193 | | |

For these experiments, human ASN sequences (i.e., DNAzyme sequences without the DNA cleavage motifs) were then transformed into rat-specific ASNs for in vivo screening. See Table 19, below.

TABLE 19
Rat-specific anti-Cx26 ASNs for in vivo screening in rat

| | | | Human- |
|---|---|---|---|
| | | sense seq (rc)-Target on | Rat |
| ASN Code | ASN sequence-Rat | rat Cx26 mRNA | mismatch |
| H26_6000 | TCGATACGGACCTTCTGGGT | ACCCAGAAGGTCCGTATCGA | 1 |
| 7 | (SEQ. ID. NO: 194) | (SEQ. ID. NO: 195) | |
| H26_4500 | TCGTAGCACACATTCTTACA | TGTAAGAATGTGTGCTACGA | 2 |
| 1 | (SEQ. ID. NO: 196) | (SEQ. ID. NO: 197) | |
| H26_7501 | TGTATTGGGACAAGGCCAGG | CCTGGCCTTGTCCCAATACA | 2 |
| 1 | (SEQ. ID. NO: 1) | (SEQ. ID. NO: 198) | |
| H26_2500 | TGTGCCCCAATCCATCTTGT | ACAAGATGGATTGGGGCACA | 2 |
| 3 | (SEQ. ID. NO: 199) | (SEQ. ID. NO: 200) | |
| H26_3501 | CATCTCCCCACACCTCCTTC | GAAGGAGGTGTGGGGAGATG | 1 |
| 1 | (SEQ. ID. NO: 201) | (SEQ. ID. NO: 202) | |
| H26_6000 | ATCTCTTCGATGTCCTTAAA | TTTAAGGACATCGAAGAGAT | 1 |
| 4 | (SEQ. ID. NO: 2) | (SEQ. ID. NO: 203) | |
| H26_4500 | ATGTGAGAGATGGGGAAGTA | TACTTCCCCATCTCTCACAT | 1 |
| 8 | (SEQ. ID. NO: 204) | (SEQ. ID. NO: 205) | |
| H26_5001 | CGGTAGGCCACGTGCATAGC | GCTATGCACGTGGCCTACCG | 1 |
| 5 | (SEQ. ID. NO: 206) | (SEQ. ID. NO: 207) | |
| H26_8500 | AGGATGCAAATTCCAGACAC | GTGTCTGGAATTTGCATCCT | 0 |
| 5 | (SEQ. ID. NO: 192) | (SEQ. ID. NO: 193) | |
| H26_8001 | AGATCATGAACACCGTGAAG | CTTCACGGTGTTCATGATCT | 3 |
| 1 | (SEQ. ID. NO: 208) | (SEQ. ID. NO: 209) | |

*Mismatches between the rat and human sequences are bolded.

### Example 6

### Anti-Cx26 Polynucleotide Testing in Rats

This example describes the testing of the rat-specific anti-Cx26 ASN polynucleotides described in Example 5, above, in rats.

### Objective

The objective of the following experiments was to determine if any of the 10 different Cx26 antisense oligonucleotides described in Example 6 showed signs of either knocking down Cx26 protein levels or effecting early re-epithelialization in vivo.

### Overview

Four to five week old Sprague-Dawley rats (80-200 g) were obtained, housed individually in cages lined with tray liners, and maintained in standard conditions. Four full thickness excisional wounds were made on the dorsum of each rat using a 6 mm punch biopsy tool, as such wounds lead to a large increase in Cx26 expression within the wound leading edge 6 hours post-wounding that is still maintained at 24 hours. The 10 different Cx26 antisense oligonucleotides to be tested were formulated as investigational drug products (IDPs) in three dose concentrations, 30, 100, or 300 μM, in pluronic gel. The IDPs were applied (25 μL) to individual wounds (each ASN polynucleotide was tested in three (n=3) animals) and the animals were allowed to recover. The rats were euthanized at 6 hours or 24 hours post-wounding. Tissue was collected for processing for quantitative assessment of Cx26 knockdown (6 hr.) and any early re-epithelialization (24 hr.).

### Investigational Drug Products

As already described, the 10 rat-specific anti-Cx26 ASN polynucleotides were derived from anti-Cx26 ASNs designed to target human Cx26 mRNA that also showed activity on rat Cx26 mRNA. The rat-specific molecules were then made by correcting for species-specific mismatches and then screened for activity against rat Cx26 mRNA. The 10 rat-specific anti-Cx26 ASN polynucleotides used to produce the IDPs are shown in Table 20, below.

TABLE 20
Rat-specific anti-Cx26 ASNs formulated to produce IDPs

| | | Corresponding |
|---|---|---|
| | | human Cx26 |
| SEQ ID NO. | Rat-specific Cx26 ASN Sequences | ASN code |
| SEQ. ID. NO.: 194 | 5′-TCGATACGGACCTTCTGGGT-3′ | H26_60007 |
| SEQ. ID. NO.: 196 | 5′-TCGTAGCACACATTCTTACA-3′ | H26_45001 |
| SEQ. ID. NO.: 1 | 5′-TGTATTGGGACAAGGCCAGG-3′ | H26_75011 |
| SEQ. ID. NO.: 199 | 5′-TGTGCCCCAATCCATCTTGT-3′ | H26_25003 |
| SEQ. ID. NO.: 201 | 5′-CATCTCCCCACACCTCCTTC-3′ | H26_35011 |
| SEQ. ID. NO.: 2 | 5′-ATCTCTTCGATGTCCTTAAA-3′ | H26_60004 |
| SEQ. ID. NO.: 204 | 5′-ATGTGAGAGATGGGGAAGTA-3′ | H26_45008 |
| SEQ. ID. NO.: 206 | 5′-CGGTAGGCCACGTGCATAGC-3′ | H26_50015 |
| SEQ. ID. NO.: 192 | 5′-AGGATGCAAATTCCAGACAC-3′ | H26_85005 |
| SEQ. ID. NO.: 208 | 5′-AGATCATGAACACCGTGAAG-3′ | H26_80011 |

Table 21 sets out the components of the IDPs made using the 10 rat-specific anti-Cx26 ASN polynucleotides listed in Table X-13, above.

TABLE 21
Investigational Drug Products (IDPs)

| | |
|---|---|
| Identity: | Connexin26 Antisense Oligonucleotide |
| | (Cx26asODN 1-10) |
| Description: | SEQ ID NOs: 194, 196, 1, 199, 201, 2, 204, |
| | 206, 192, or 208. |
| Anti-Cx26 ASN | See Table 20, above |
| Concentration: | Each Anti-Cx26 ASN was formulated in |
| | concentrations of 30, 100, and 300 μM |
| Purity: | Anti-Cx26 ASN supplied desalted but at unknown |
| | purity. Assumed 100% pure for calculations. |
| Storage | Frozen (−20° C. +/− 5° C.) |
| Conditions: | |
| Vehicle | Poloxamer Vehicle (Sigma) |
| Vehicle | approximately 27% |
| Concentration | |

### Wounding Protocol

Prior to surgery each rat (60 in total) was injected subcutaneously with buprenorphine (0.1-0.5 mg/kg SC). Anesthesia was induced with 4% isoflurane, 20% Oxygen, and 10% Nitrous Oxide maintained with 1.5% isoflurane. Following the operation rats were kept anaesthetized for at least 30 minutes to allow penetration of the IDP being tested.

Rats were randomly allocated into the 10 different treatment cohorts (Cx26asODN 1-10) and then further subdivided into two time points, 6 hours and 24 hours post-wounding. Three rats were used for each antisense at three concentrations per time point.

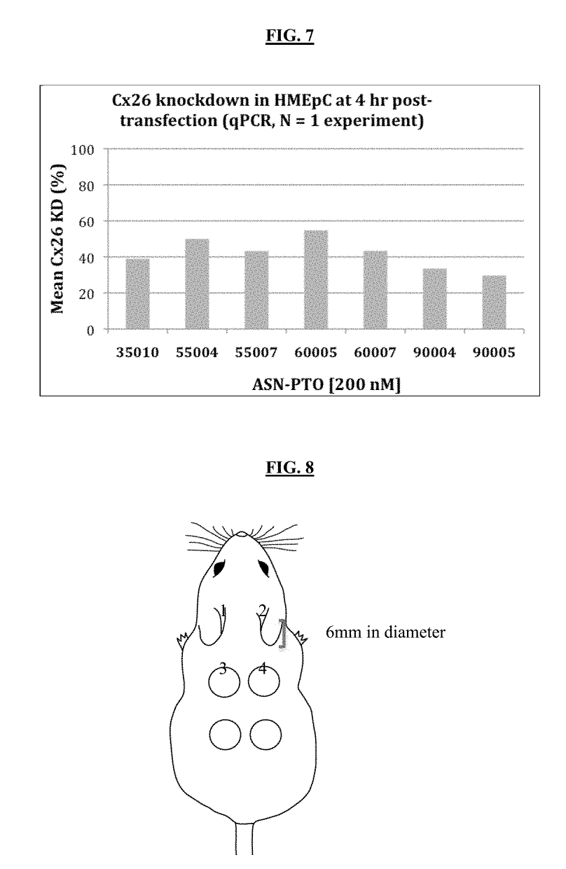

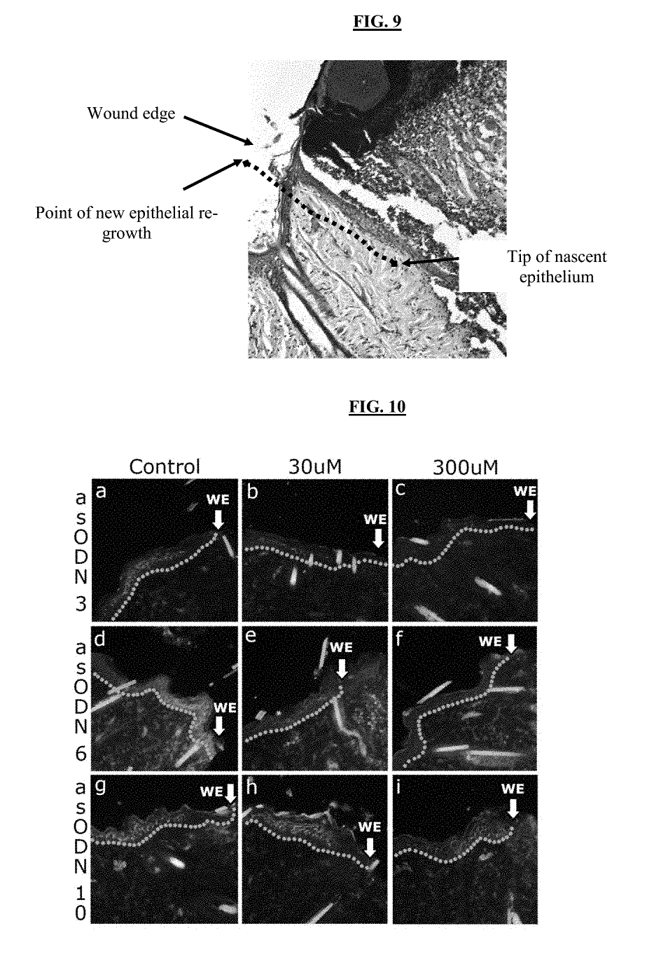

Under anesthesia rats were shaved with clippers and a large patch of fur removed from its dorsum. Four 6 mm (diameter) circular wounds were made on the shaved dorsal region of each rat. Each excision was approximately 1.0 cm apart and 1.0 cm from the mid-line of the spine. See FIG. 8. Full thickness excisions were made with a sterile 6 mm punch biopsy tool and a clean tissue paper swab was used to absorb any excess blood from the wound site. After bleeding had stopped, 25 μL of the IDP (Cx26asODN 1-10) was applied following the dose regimen described in Table 22, below.

TABLE 22
IDP Dosing Schedule and Wound locations for each test ASN*

| | |
|---|---|
| Wound Position | Delivered |

Rat 1

| | |
|---|---|
| 1 | Pluronic |
| 2 | 30 μM |
| 3 | 100 μM |
| 4 | 300 μM |

Rat 2

| | |
|---|---|
| 1 | 100 μM |
| 2 | Pluronic |
| 3 | 300 μM |
| 4 | 30 μM |

Rat 3

| | |
|---|---|
| 1 | 300 μM |
| 2 | 100 μM |
| 3 | 30 μM |
| 4 | Pluronic |

*Location according to wound numbers in FIG. 8.

### Dosing

The 60 rats used were subdivided into 3 separate treatment groups. 25 μL of each test article (IDP into each wound) was applied. Each of the 3 rats had 1 of the 10 previously specified IDPs applied at all three concentrations, as well as pluronic vehicle control to different allocated dorsal wound sites on each individual rat. Each IDP was applied to the wounds as a chilled (4° C.) liquid and allowed to gel. Following IDP application, anesthesia/sedation was maintained for at least 30 minutes whilst each rat was monitored closely. Rats in the 6 and 24 hr. cohorts were humanely euthanized at 6 hr. and 24 hr. after IDP application.

### Assessment of Cx26 Knockdown

After the rats were euthanized, wounds were collected and sectioned for qPCR and immunohistochemistry to assess Cx26 knockdown in the 6 and 24 hr. wounds and histological analysis to assess re-epithelialization in the 24 hr. wounds. To assess Cx26 knockdown, slides were examined using a fluorescent microscope and comparisons were made within each animal compared to the vehicle control. Measurement of re-epithelial growth was made using the digital images, as well, and all wounds where the histological preparation was judged to be adequate on the H&E stained slide also had the distance from the top of the junction between old and new epithelium to the tip of the nascent epithelium measured on each side, as shown in FIG. 9.

The assessment of each wound was conducted by an experienced wound assessor blinded to the treatment dose allocation. A minimum of two individual epithelial re-growth measurements was taken from the histological sections of each wound in order to derive an average for that wound site.

Results: Part A—Immunohistochemistry at 6 hr. Post-Treatment

FIG. 10 shows the expression and localization of rat Cx26 at the wound edge of 6 mm wounds 6 hr. post-treatment. As shown in FIG. 10, Cx26 staining is localised to the epidermis outlining the cellular membranes. All other visible tissue is seen solely due to the autofluoroescent nature of the samples. All images for each individual antisense oligodeoxynucleotide (asODN) treatment, i.e., three, come from the same animal. Photos a, d, and g show the results for administration of the vehicle-only control (i.e. pluronic vehicle-only treated wounds). Photos b and c show wounds treated with 30 μM or 300 μM of Cx26asODN 3, respectively. Photos e and f show wounds treated with 30 μM or 300 μM of Cx26asODN 6, respectively. Photos h and I show wounds treated with 30 μM or 300 μM of Cx26asODN 10, respectively. With asODN 3 and 6, a clear decline in Cx26 expression was evident with increasing dose when compared directly to pluronic treated, control tissue, while after treatment with asODN 10, high levels of Cx26 expression persisted (i.e., no effect). For human subjects doses any dose or concentration of anti-connexin agent described herein may be used.

Results: Part B—Wound Healing at 24 hr. Post-Treatment

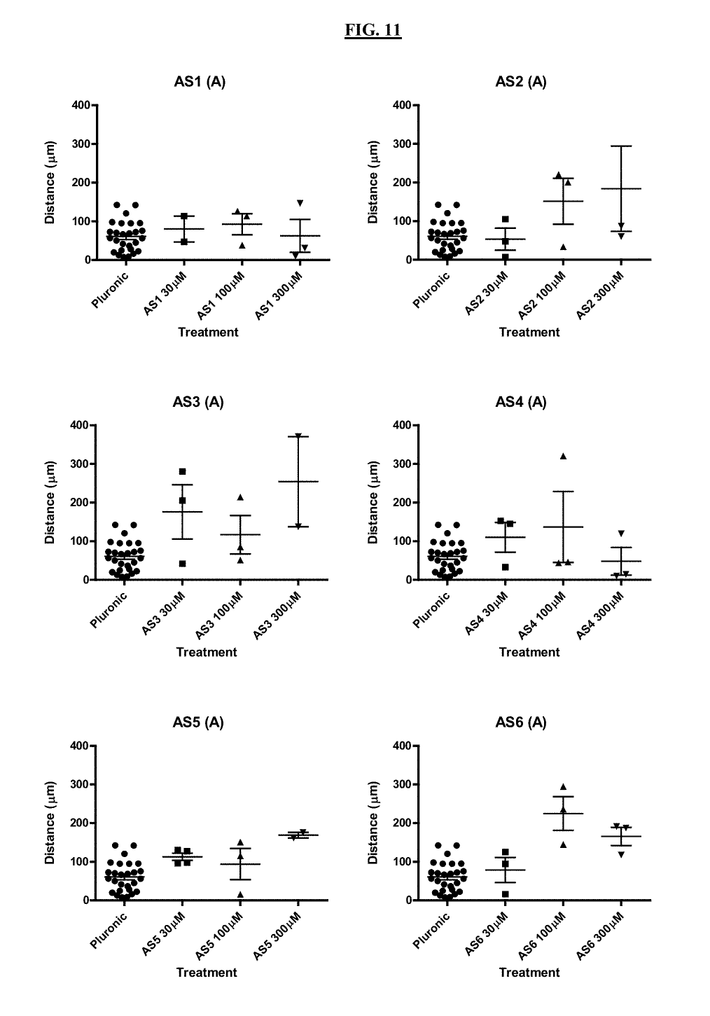

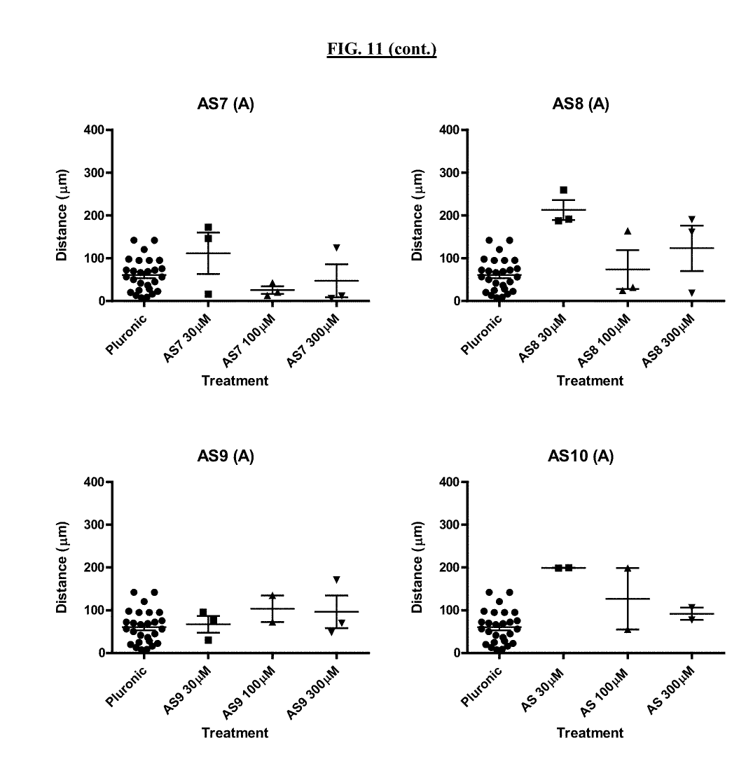

FIG. 11 shows data as the group mean±SEM of the three rats given a particular anti-Cx26 ASN polynucleotide. Each data point shown was one measurement from each side of the wound that was averaged to give a single value for that rat wound. The vehicle wounds were also the mean of two measurements on each wound with all the study vehicle wounds then grouped together to show the scatter in the wider vehicle-only control group.

The degree of re-epithelialisation (distance in μm) seen in wounds treated with the pluronic (vehicle)-only control, 30 μM, 100 μM, or 300 μM (vehicle) of the specified test agent antisense oligodeoxynucleotide (asODN) after 24 hours post-wounding and treatment. The pluronic control group displayed in each graph is composed of all wounds exposed to this treatment within the study (n=30), while each specified asODN treatment has a minimum n=2.

### Example 7

### Dosage Forms for Delivery of Anti-Connexin Agents

Anti-connexin agents were also formulated in dosage forms suitable for sustained administration in which one or more compositions comprising one or more anti-connexin agents were coated on a scaffold core. The release properties of different formulations of coatings on a collagen scaffold were examined to try to generate a coating that would release a Cx43asODN at a steady rate over a period of days. The inventors sought to create a sustained release of Cx43asODN which would (1) promote sustained re-epithelialization, by downregulating Cx43 at the wound edge throughout the multi-day window, and (2) additionally reduce the inflammation and foreign body response, as manifested by neutrophil cell infiltration and abnormal epithelial wound edge thickening near the scaffold.

### Scaffold Formation

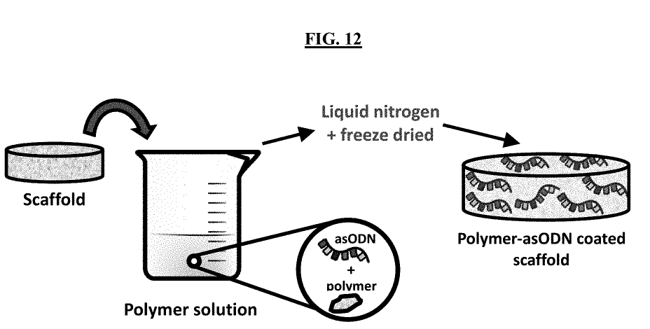

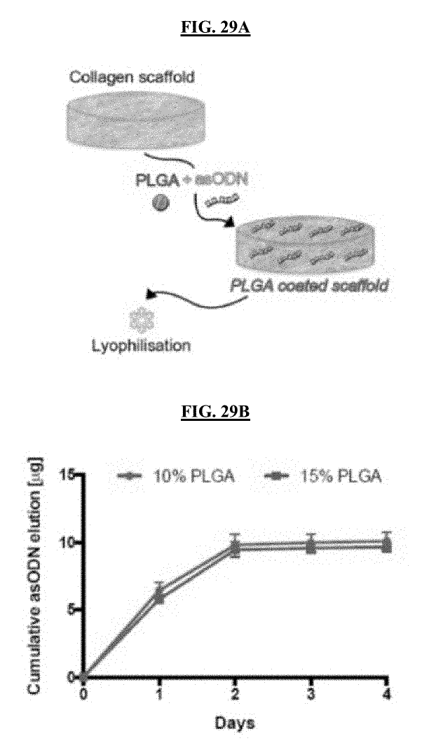

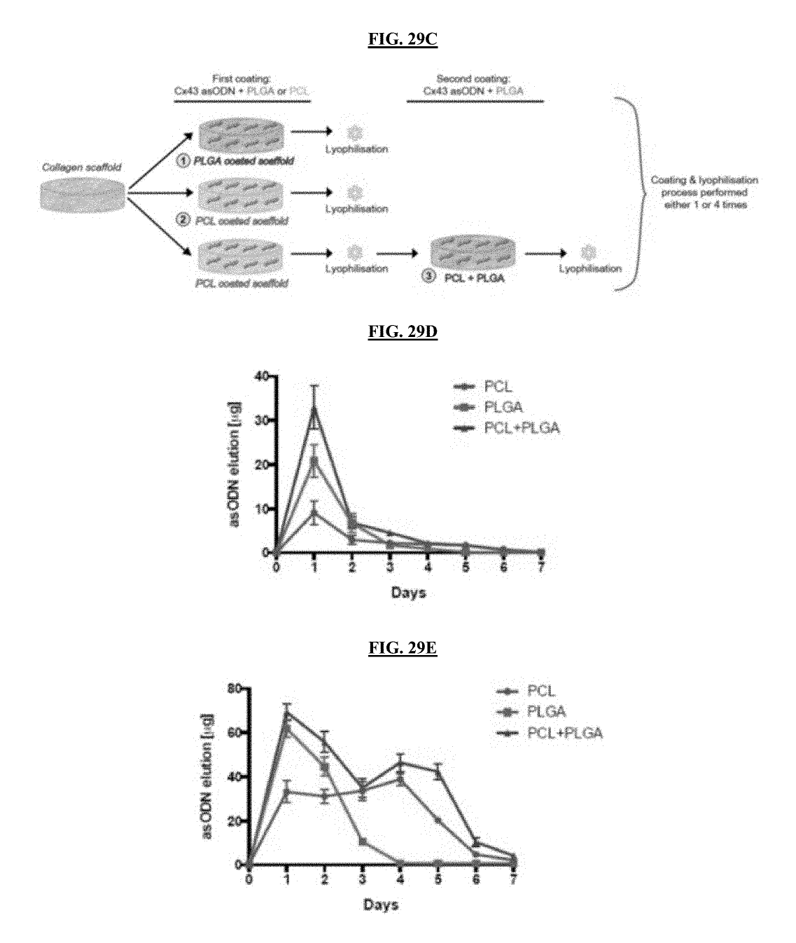

Scaffolds were initially coated using either a 10% or 15% (wt./v.) solution of PLGA and asODN 100 μM (FIG. 12). Scaffolds were then submerged in 40 μl nuclease free water and assayed for DNA elution at daily intervals for 4 days (FIG. 29B). By the D2 time point over 95% (approximately 10 μg) of DNA had eluted for both 10% and 15% PLGA coated scaffolds. Exemplary wounds of 6 mm in diameter, depth 0.7 mm would have a volume of 19.8 mm³. DNA elution from both the 10% and 15% PLGA coatings was calculated to produce an effective asODN concentration of 54.4 μM in the wound bed.

In order to both prolong the duration and quantity of asODN release, the asODN loading concentration was increased to 300 μM. Three new combinations were produced for evaluation: i) scaffolds coated using just 15% PLGA and the Cx43asODN (a form of a “single-coated scaffold”), ii) scaffolds coated in 10% PCL and Cx43asODN (another form of a “single-coated” scaffold) or iii) a combined approach of a layer of PCL+Cx43asODN, followed by a layer of PLGA+Cx43asODN (FIG. 29C) (“double-coated” scaffolds, which can have one or more layers of each type of coating). Elution profiles were then studied over a 7-day period (FIG. 29D). For single coating of PCL, elution of asODN occurred more gradually than from the single PLGA coating. The PCL-only group, eluted 19 μg of DNA in 7 days, with half released after 1 day, as compared to 15 h for half of the DNA from the single PLGA coating. Scaffolds with a combination of PCL and PLGA coatings eluted approximately 48 μg of asODN over the course of 7 days of which half eluted within 24 h. Elution of 48 μg released at once into a typical 19.8 mm3 wound volume, was calculated to produce an effective concentration of 260.4 μM.

For PCL only scaffolds with four coatings of PCL, half of the asODN eluted by 2.5 days, relative to just 1 day for a scaffold with a single PCL coating. The most noticeable change in performance, however, was found using scaffolds coated with four layers of the PCL/asODN coating each applied by the dip/lyophilization procedure, followed by four layers of the PLGA/asODN coating. This coating resulted in an average 7-day total release of 265 μg, which was considerably greater than the coatings of only PCL or PLGA. When asODN elution from combination-coated scaffolds was assessed on a day-by-day basis as opposed to cumulative release, there were two clear peaks of asODN elution in the first 24 hours and then between D3 and D4 (FIG. 29E). This indicates the combination-coated scaffolds exhibited a sustained release of the asODN.

The results from the elution assays were unexpectedly superior and identified the 4-layered combined PCL and PLGA coating as suitable for testing of function in vivo.

In one experiment, scaffold fabrication was obtained by the following method: collagen scaffolds were fabricated by electrospinning acid-soluble bovine collagen (>99% purity, Kensey Nash, Pa., USA) blended with poly-ε-caprolactone (>99% purity, Sigma Aldrich, Poole, UK) at a 10:1 weight ratio, respectively. Blended polymers were dissolved in hexafluoropropan-2-ol (HFP, 99% purity, Apollo Scientific, UK) at 10% (wt./v.). The number average (Mn) molecular weight of the poly-ε-caprolactone was 2,000. The poly-ε-caprolactone Mn can be from 450 to 80,000, preferably between 500 and 2,000. The general electrospinning process has been described previously (Torres-Giner, S., et al., (2009), ACS Appl Mater Interfaces 1, 218-23). Scaffolds were electrospun at a flow rate of 5 ml/hour, with a needle to collector distance of 130 mm and using an output voltage of 13 kV. 14 ml of polymer solution was spun onto a 9 cm² foil sheet to produce scaffolds with an average thickness of 0.4 mm. To generate scaffolds with an average thickness 0.8 mm, 30 ml of polymer solution was electrospun. Uncoated electrospun scaffolds were crossl-inked by immersion in either a high (15% wt./v.) or a low (0.15% wt./v.) concentration of N-(3-Dimethylaminopropyl)-N′-ethylcarbodiimide (EDC) hydrochloride (99.5% purity, Apollo) in a 1:10 water to acetone solution. Scaffolds were washed for 20 min with sterile PBS, sterilised for 1 h using 70% ethanol, and washed 3 times with sterile PBS. Scaffold discs were then punched out using a 6 mm biopsy punch to match the size and shape of excisional wounds.

Application of Polymer Coatings Containing Cx43 asODN to Scaffolds

The rat Cx43asODN sequence 5′-GTA ATT GCG GCA GGA GGA ATT GTT TCT GTC-3′(SEQ ID NO:5). A non-functional sense (sODN) sequence 5′-GAC AGA AAC AAT TCC TCC TGC CGC AAT TAC-3′ (SEQ ID NO:210). A modified version of the Cx43 asODN sequence had the fluorophores Cy3 and Cy5 conjugated at the 5′ and 3′ ends, respectively. Either PCL or Poly(D, L-lactice-co-glycolide) (PLGA) was dissolved in dimethyl carbonate at 10% wt./v. The asODN sequences were dissolved in water and mixed with the polymer solutions to either a 100 μM or 300 μM final DNA concentration. The two immiscible layers were processed for 10 seconds using a handheld homogenizer on a medium setting at room temperature to produce an emulsion. After preparing the coating mixture, individual 6 mm collagen scaffolds were dipped for 1 second in the desired polymer-asODN solutions and immediately placed in a tube of liquid nitrogen and lyophilised overnight to remove the solvent from the scaffolds. In some experiments this coating process was repeated 3 times to produce 4 layers of polymer+asODN, or 7 times in the case of ‘double-coated’ scaffolds, which received 4 coating layers of PCL+asODN followed by 4 layers of PLGA+asODN.

Quantification of asODN Elution

Three coated scaffolds were immersed in 30 μl of nuclease free water at 37° C. At specified intervals, 1.5 μl of liquid was removed and assayed in a NanoDrop UV spectrophotometer under the ssDNA setting to determine DNA concentration. Scaffolds were placed in a fresh 30 μl of water to compensate for the reduction in volume following water removal for assays. Individual time point elution data was collected to produce both cumulative and daily asODN elution graphs.

### Förster Resonance Energy Transfer Analysis

The integrity of asODN incorporated into scaffolds was assessed using Förster Resonance Energy Transfer (FRET) imaging. Scaffolds were coated with Cx43 asODN conjugated at either end to fluorophores Cy3 and Cy5. Scaffolds were either left untreated or submerged in 37° C. foetal bovine serum (FBS) for up to 7 days. Samples were removed every 24 h then snap frozen in TissueTek O.C.T. medium (Sakura Finetek, UK), and sectioned to a thickness of 10 μm using a Leica cryostat. Scaffold sections were then washed briefly with PBS and mounted with a size 1 coverslip using Citifluor AF1 mountant (Citfluor Ltd, UK). Samples were imaged using a Leica SP8 confocal microscope using the FRET wizard. Six regions were selected at random for acceptor fluorophore (Cy5) bleaching using a 633 nm laser. A marked increase in Cy3 signal emission at 570 nm post-bleaching was considered to be indicative of FRET activity and was used to calculate a FRET efficiency. Images captured were 8-bit single optical sections with a resolution of 1024 by 1024 pixels. To determine the potency of serum in degrading asODN the wavelength emission spectra of 6 asODN regions per time point were generated by performing xyλ-mode scans on FRET labelled asODN that had been incubated in FBS at 37° C. Individual λ-scans were performed using a 543 nm laser excitation, with 19 equally sized detection steps occurring between the wavelengths of 550 nm to 730 nm. Emission values were output using the LAS AF software.

### Surgery

Sprague Dawley rats were reared on site at the BSU and used at 6 weeks old. Rats were anaesthetised using 4% isoflurane, 20% oxygen and 10% nitrous oxide, and maintained using 1.5% isoflurane. Animals were injected subcutaneously with 0.03 mg/ml buprenorphine (Vetergesic) before operation. Rat backs were shaved and covered in a thin layer of Nair® hair removal cream, after which both the cream and hair was removed using a warm moistened gauze pad. Animals were placed on heated mats and four full-thickness excisional 6 mm biopsy punch wounds were made two on each side of the dorsal midline. Scaffold treatments were applied directly to wounds, after which the back was covered with a sheet of Tegaderm™ film. Post-procedure, animals were kept in a heated chamber and monitored for recovery.

### Harvesting Wounds

Animals were culled by cervical dislocation at 1, 3, 5, 10 or 15 days post wounding (N=8 per time point). Wounds were macroscopically imaged with a Leica MZ8 dissection microscope and then excised and fixed in 4% paraformaldehyde for 24 h at 4° C. and transferred to 20% sucrose overnight, washed in PBS then bisected. Half of each wound was snap frozen in O.C.T. medium, then sectioned using a cryostat at a thickness of either 5 μm (for H&E staining) or 10 μm (for immunofluorescence staining).

### Haematoxylin and Eosin Staining

Frozen tissue sections were thawed then covered completely in tap water for 5 min at room temperature. Slides were submerged in Harris' haematoxylin solution for 30 seconds followed by washing in running tap water for 5 min. Background staining was reduced by dipping the slides in 1% acid alcohol (1% glacial hydrochloric acid, 70% ethanol, 29% distilled water) for 3 seconds, followed by running tap water for a further 5 min. Samples were then submerged for 15 seconds in eosin B solution, followed by 5 seconds in a tap water, then 70% ethanol for 2 min, 100% ethanol for 2 min and a second 100% ethanol for a further 2 min, to dehydrate the sections followed by 2× xylene for 5 min. Slides were then mounted in a xylene based DPX mountant and coverslipped.

### Immunofluorescence Staining

Tissue sections were permeabilised in cold acetone for 5 min and then blocked using a 0.1 M lysine PBS solution containing 0.1% Triton-X-100. Sections were stained for 1 h using a rabbit polyclonal antibody to Cx43 (1:2000 dilution, C6219, Sigma Aldrich UK) or Cx26 Cx26 (1:200 dilution, (Diez et al., 1999). Sections were then stained using a goat anti-rabbit Alexa 488 secondary antibody (A11008, Invitrogen UK) at a 1:400 dilution for 1 h. Tissue was counterstained using Hoechst solution (both Hoechst 33528 and Hoechst 33342 dyes at 1:50,000 dilution, Sigma Aldrich UK). Slides were mounted in citifluor and coverslipped.

### Epidermal Measurements

H&E stained sections were examined using a Leica DMLB light microscope and images were captured at either 10× or 20× magnification. Epidermal thickness was determined by measuring the thickest point along the basal to spinous layer axis within the end 100 μm of nascent epidermis using ImageJ. The length of nascent epidermis outgrowing from the wound margin was measured at either side of the wound using ImageJ and averaged to return a re-epithelialisation distance measurement for each sample.

### Polymorphonuclear Cell Quantification

H&E stained tissue sections were examined using a light microscope and polymorphonuclear cells (PMNs) were identified 3 fields of view from the wound edge in the dermis using a 40× objective. Individual images of this region for each sample were then captured using a 20× objective. The numbers of PMNs in this area was quantified manually with assistance of a counter tool in ImageJ.

### Measurement of Granulation Tissue Area Smooth Muscle Actin Area

Rat wounds harvested at D10 and D15 were H&E stained and imaged with a Zeiss Axio Scan.Z1 automated slide scanner. Brightfield images were captured using a 20× objective then automatically stitched together to form a montage and quantified for granulation tissue area using ImageJ.

### Confocal Microscopy and Image Analysis

Single optical section images of Cx43 or Cx26 immunostained tissue sections were acquired using a Leica SP2 confocal microscope with a 40× oil objective. 8-bit images were acquired 1024 by 1024 pixels. Image acquisition settings remained identical between each treatment in order to allow comparisons to be made during image analysis. Connexin protein expression was quantified by counting positive pixels on binary images of wound edges with ImageJ software using the method outlined in a study by (Wang et al., 2007). Images from each experiment were identically thresholded. Only the number of connexin positive pixels contained within the end 150 μm of the nascent epidermis was assessed in order to gauge only protein expression at the epidermal wound edge. Connexin levels were expressed per square micron of epidermis to compensate for variations in epidermal thickness between samples. In order to account for variations in connexin levels between individual animals, wound edge expression was normalised to connexin levels at a distal site away from the wound edge and expressed as a percentage change from distal levels. In the case of Cx26 stained tissue, distal levels were extremely low, to the extent that small fluctuations between samples could result in large differences between values. For this reason, Cx26 distal levels were averaged and these values were used to calculate the change in wound edge Cx26 expression.

### Statistical Analysis

All data amenable to statistical analysis was subjected to Kolmogorov-Smirnov tests to ascertain normality. Normally distributed data was then subjected to parametric statistical tests. Other data or multiple comparisons was subjected to a one-way ANOVA test followed by a Tukey's post-hoc test to investigate individual statistical significance differences. GraphPad Prism 5.0 software was used to perform all statistical analyses.

In one experiment, scaffold fabrication was obtained by the following method: a mixture of 95% collagen and 5% PCL was electrospun into fibers to create a sheet of scaffold, and 6 mm scaffold discs were punched out of the sheet using a biopsy punch to obtain a disc-shaped scaffold. The polymer coating was applied by the following process (depicted in FIG. 12):
1. Antisense oligonucleotides (asODN) were dissolved in MilliQ water such that the final concentration (solvent plus aqueous DNA solution) for any given asODN sequence was 300 uM, although any concentration described herein may also be used.
   The polymer (poly(lactic-co-glycolic acid (PLGA) or Polycaprolactone (PCL) was dissolved in the organic solvent dimethyl carbonate at 10% (PCL) or 15% (PLGA) wt/v. In a separate experiment, the concentration of the PLGA was 10% or 15% (wt/v).
   The aqueous and organic solutions were combined. Solutions were homogenised using a handheld homogeniser at a 1 in 10 volume ratio (DNA solution to water) to dissolve or suspend both the asODN and the polymer.
2. The scaffold discs were then briefly submerged in the homogenised solution for about 5 seconds, then removed from the solution and immediately submerged in liquid nitrogen to freeze the coated scaffold structure. The frozen species was then freeze dried to remove the solvent or solvents. This process constituted 1 dip.
3. This dipping and freeze drying process was performed multiple times in the following order:

TABLE 23
Order and number of scaffold dips in preparing asODN-comprising
polymer-coated scaffold. Each dip forms a layer of the coating
comprising the indiciated polymer and anti-connexin agent.

| Polymer | Anti connexin agent | Number of dips + freeze dries |
|---|---|---|
| PCL | asODN | 1 times |
| PLGA | asODN | 1 times |
| PCL | asODN | 4 times |
| PLGA | asODN | 4 times |

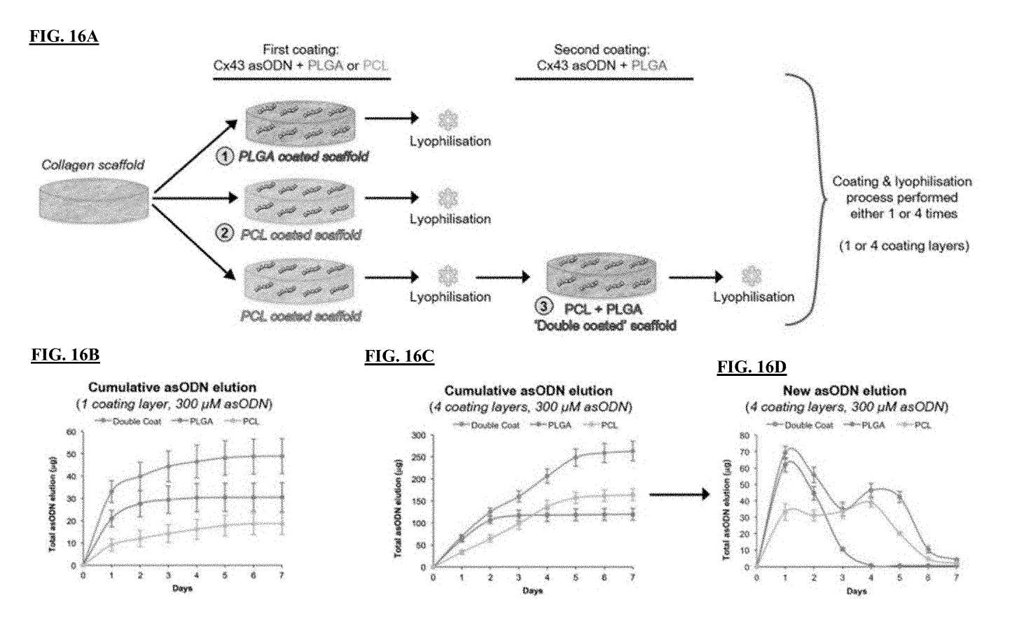

4. A “double-coated” scaffold was prepared using more than one coating, each comprising either PCL or PLGA polymer. This was prepared by applying a PCL/asODN layer by the dip/lyophilization procedure, followed by a PLGA/asODN coating by the dip/lyophilization procedure. A second “double-coated” scaffold was coated by four layers of the PCL/asODN coating each applied by the dip/lyophilization procedure, followed by four layers of the PLGA/asODN coating, as outlined in FIG. 12 and FIG. 16A.

### Materials:

The PCL (PN 440744), PLGA 65:35 (PN P2066), and Dimethyl carbonate ‘DMC’ (PN 517127) were obtained from Sigma-Aldrich. Acid soluble bovine collagen may be used. Bovine collagen is also available from Sigma-Aldrich.

### ASN Sequences

TABLE 24
The Cx26 ODN and Cx 43 ODN sequences used in coating

| SEQ. ID. NO. | Nomenclature | Sequence (rodent version of human sequence) |
|---|---|---|
| SEQ. ID. NO. 1 | Cx26 asODN | TGTATTGGGACAAGGCCAGG |
| | Antisense Sequence 1 | |
| SEQ. ID. NO. 2 | Cx26 asODN | ATCTCTTCGATGTCCTTAAA |
| | Antisense Sequence 2 | |
| SEQ. ID. NO .5 | Cx43asODN | GTA ATT GCG GCA GGA GGA ATT GTT TCT GTC |
| | Antisense | |
| SEQ. ID. NO. 210 | Cx43senseODN | GAC AGA AAC AAT TCC TCC TGC CGC AAT TAC |
| | Sense | |

Both Cx26 asODN sequences were individually dissolved in MilliQ water and mixed with each polymer solution at a final concentration of 300 micromolar. In between dips, scaffolds were dipped in liquid N₂ and lyophilized overnight to remove the solvent or solvents.

All Cx26-coated scaffolds contained both sequences at 300 uM concentration each. Cx26 sequences were combined on each scaffold coating.

The sustained release of an anti-connexin agent from a coated scaffold was demonstrated for two different PLGA concentrations in the dip solution. A single coating of the scaffold was made using 100 uM of asODN in water/dimethyl carbonate suspension using 10% PLGA, or separately 15% PLGA. After freeze-drying of the coated scaffold, the coated scaffold was placed in solution for several days and samples of the solution were taken to measure the amount of asODN released, FIG. 13. Each data point represents n=3 samples, with the max and min and average values shown on the graph. FIG. 13 shows the cumulative eluted asODN profile from the single coated scaffold over time, showing that the amount released is initially low, but increases to about the maximum amount released at about 2 days.

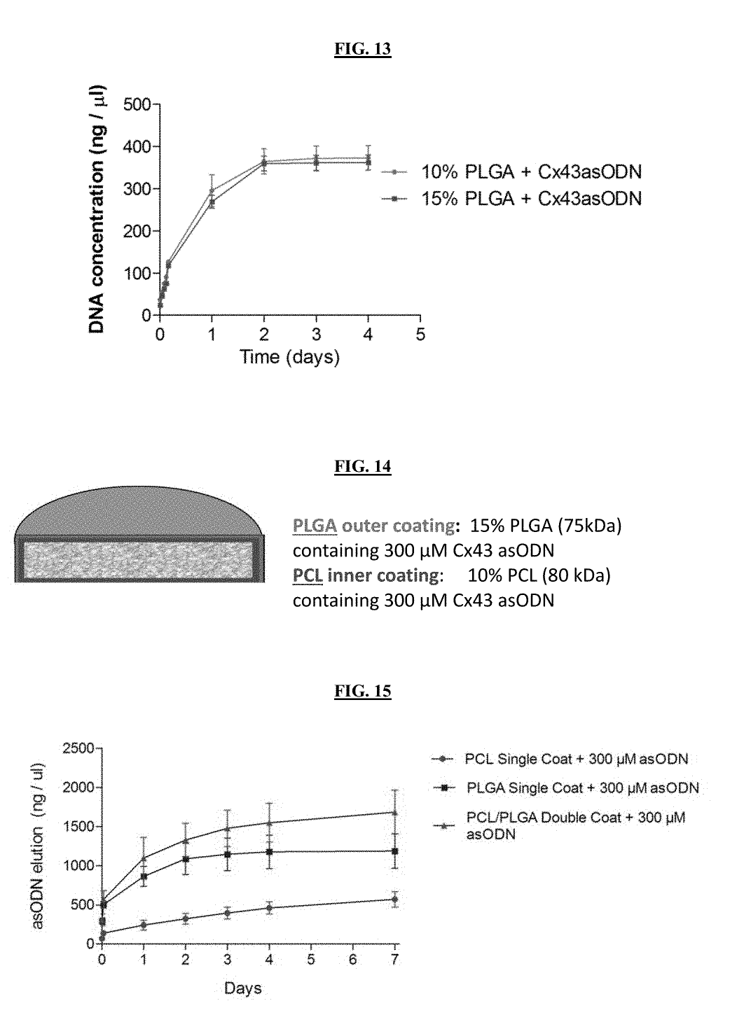

To control the release of the anti-connexin agent from a coated scaffold, multiple layers of the anti-connexin agent were applied to the scaffold core using techniques similar to that described above. First, a single PCL layer comprising Cx43 asODN was applied using 10% PCL (Mₙ of 80,000 Da) and 300 micromolar Cx43 asODN. After immersion and freeze drying, a second coating was made using 15% PLGA (Mₙ of 75,000 Da) and 300 micromolar Cx43 asODN. As shown in FIG. 14, the coated scaffold comprised two coatings. Samples containing scaffolds with either a single layer coating each of PCL and 300 micromolar asODN, and PLGA and 300 micromolar asODN, or with four layers each of PCL and 300 micromolar asODN and PLGA and 300 micromolar asODN were made in a similar manner, as shown in FIG. 16A.

Using Coated Scaffolds to Deliver Cx43 asODN to Wounds

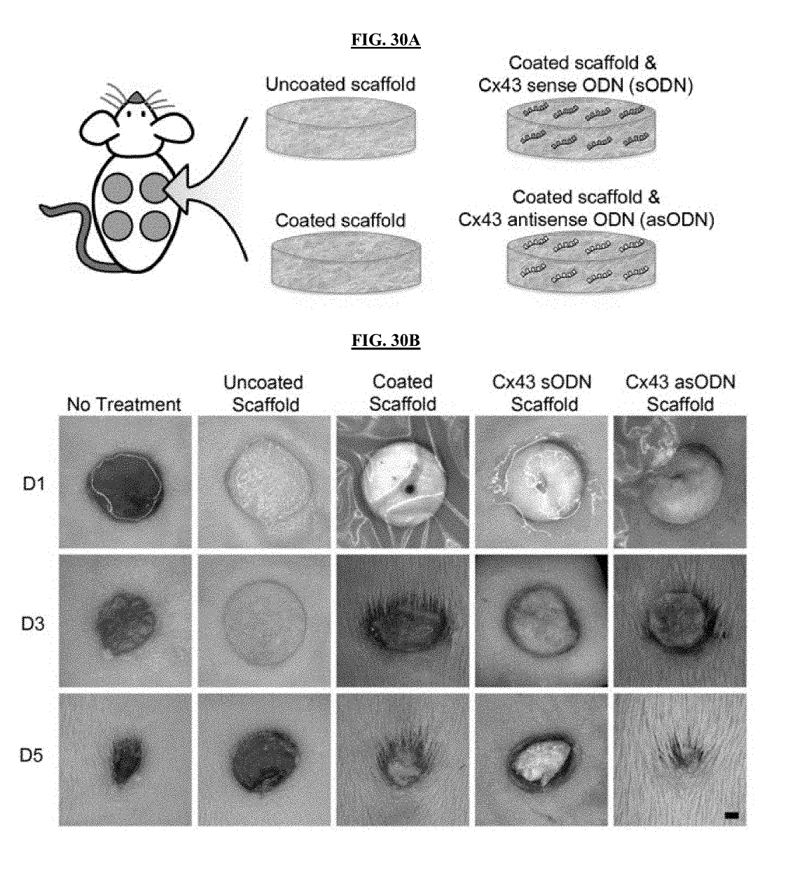

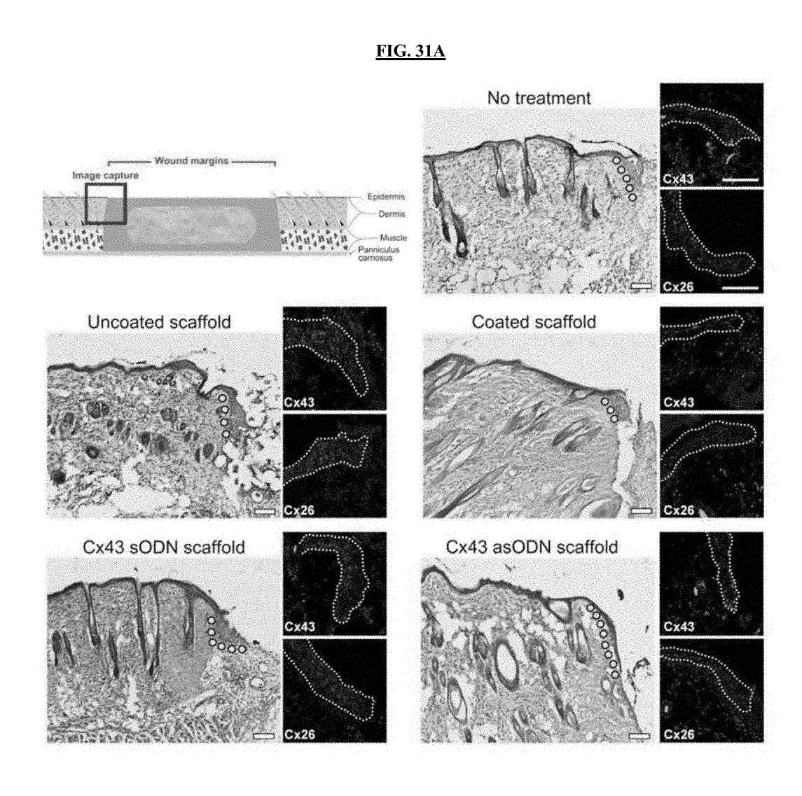

In order to compare the in vivo effects of Cx43asODN in the polymer coated scaffolds accurately, a series of control scaffolds were devised to allow comparisons (FIG. 30A). These consisted of an uncoated scaffold, as well as two additional polymer coated scaffolds, one without asODN (‘coated only scaffold’) and the other with a sense sequence to Cx43senseODN (‘Cx43senseODN scaffold’). Full-thickness 6 mm wounds were made to the backs of Sprague-Dawley rats and individual scaffolds were applied (n=8 per treatment and time point).

Wounds at D1, 3 and 5 that received uncoated scaffolds were typically larger than any of the other treatments (FIG. 30B). At D1, all scaffolds appeared to prevent wound contraction to some degree. However, by D3 wounds treated with Cx43asODN scaffolds appeared markedly reduced in size compared to the control scaffolds and were similar to untreated wounds. Wounds treated with uncoated scaffolds at D3 were similar in size to the initial wound. Coated scaffolds typically showed signs of breakdown to varying degrees at both D3 and D5, but wound edges did not appear to integrate with coated scaffolds. By D5, wounds treated with the Cx43asODN scaffolds had reduced in size considerably, often to a greater degree than untreated wounds.

### Re-epithelialisation and Connexin Expression Analysis

### Day 1

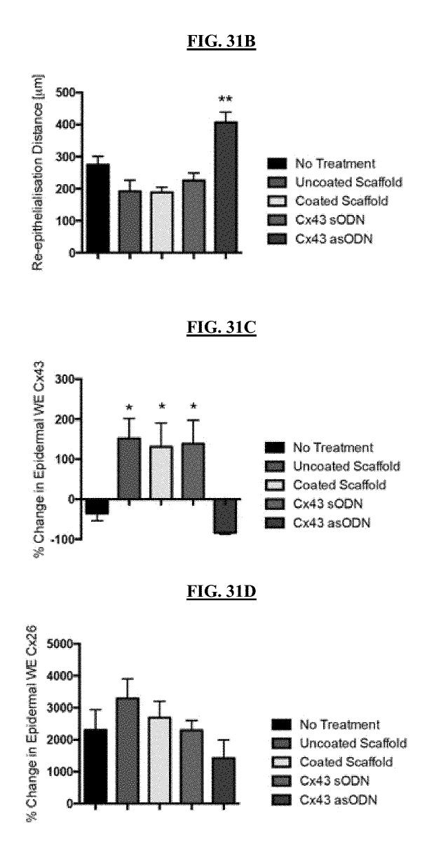

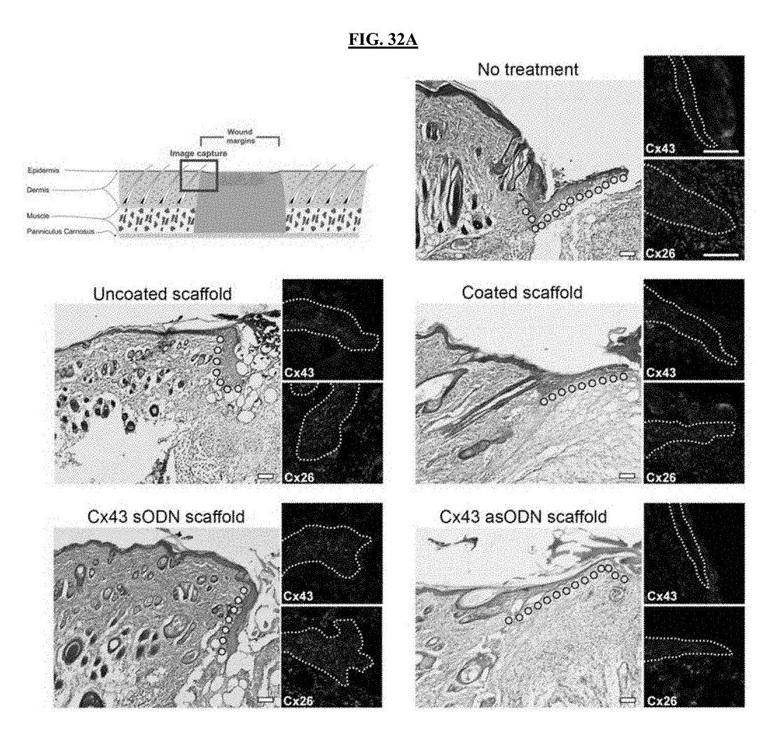

At D1, untreated wounds had re-epithelialised on average a distance of 274 μm (FIG. 31B). This was greater than control scaffold-treated wounds (uncoated, coating only, and coated+Cx43senseODN), which had re-epithelialised on average a distance of 202 μm, although the difference was not significant. Wounds treated with scaffolds coated in Cx43asODN, however, had re-epithelialised significantly further, an average distance of 407 μm, i.e. 67% greater than untreated wounds (P<0.01) and 101% greater than the combined averages of the control scaffold-treated wounds.

Sections of wound edge tissues were immunostained for Cx43 and Cx26 and protein levels of connexins quantified and normalised to a region of distal epidermis. At D1 there was a strong upregulation of Cx26 in wound edge keratinocytes across all conditions, including wounds left untreated, with no significant differences observed between groups (FIG. 31A). In Cx43asODN coated scaffolds, wound edge epidermis Cx26 was 38% lower than untreated wounds and 57% less than the average of all three control scaffold-treated wounds (FIG. 31D). Staining for Cx43 revealed a different outcome. At D1, application of each of the control scaffolds resulted in an increase in Cx43 expression in the wound edge epidermis, with an average increase of 141% over distal levels (FIG. 31C). Untreated wound edges displayed a typical downregulation of Cx43 expression by 35%, while Cx43asODN coated scaffold wound edge expression was reduced by 85%. All three control scaffold treatments showed significantly more Cx43 than the untreated control wounds (P<0.05).

### Day 3

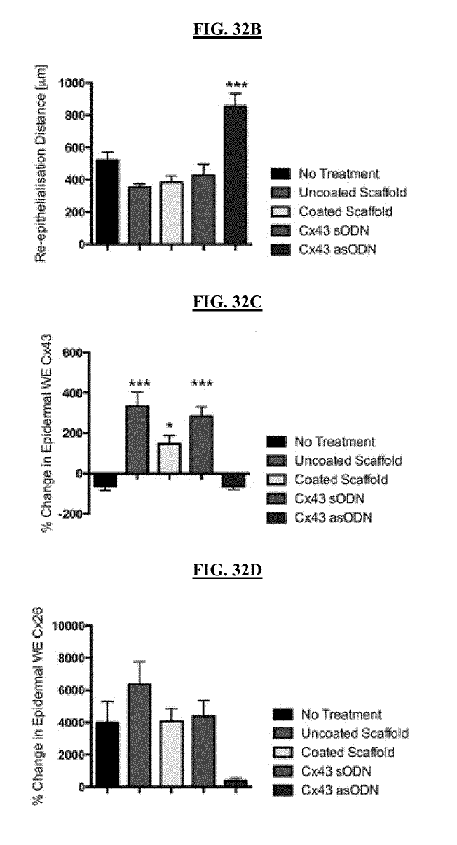

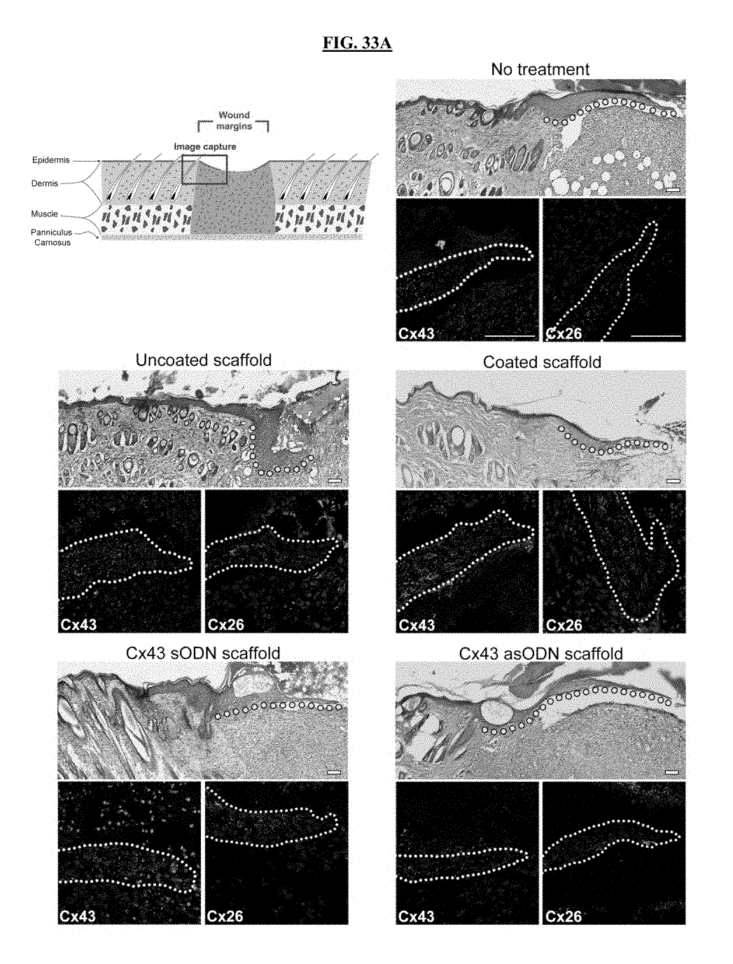

At D3, untreated wounds had re-epithelialised an average of 522 μm, whilst the average was 389 μm for the combined distances of wounds treated with control scaffolds (FIGS. 32A & B). Wounds treated with the Cx43asODN scaffolds had again re-epithelialised an average distance of 859 μm, 65% greater than untreated wounds (P<0.001) and 121% greater than the averaged distance for all control treated wounds.

At D3, wound edge keratinocyte Cx26 expression across all treatments was upregulated from the minimal levels observed in uninjured distal epidermis (FIGS. 32A & D). Uncoated scaffolds produced the highest increase in wound edge Cx26 expression, at 6370%. Cx26 levels were similar between the coated control scaffolds and untreated wounds. Wounds treated with Cx43asODN scaffolds resulted in lower Cx26 expression of only 375% increase at the wound edge at D3 but due to the large variation seen in untreated controls this did not reach significance. Cx43 expression at D3 showed a 65% downregulation in wound edges treated with Cx43asODN scaffolds, comparable to untreated wounds FIGS. 32A & C). There was a significant elevation in Cx43 protein levels in all the control scaffold groups compared to the untreated control; uncoated scaffolds (335% increase, P<0.001) and Cx43senseODN coated scaffolds (283% increase, P<0.001), un-coated scaffolds (P<0.05).

### Day 5

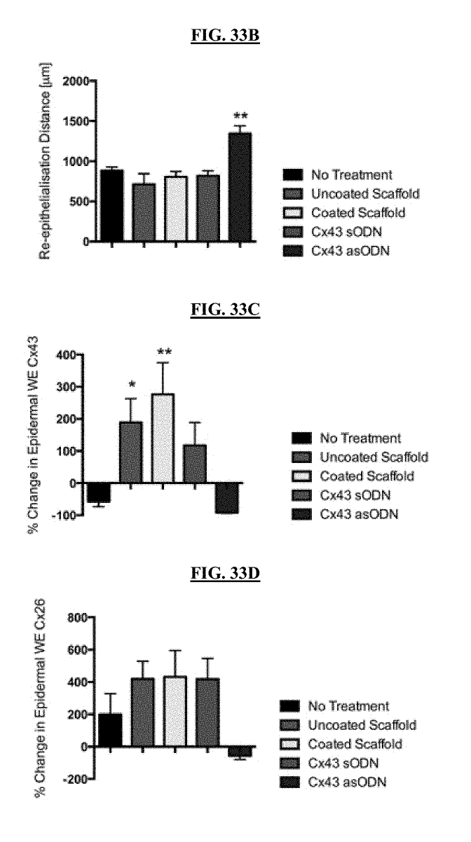

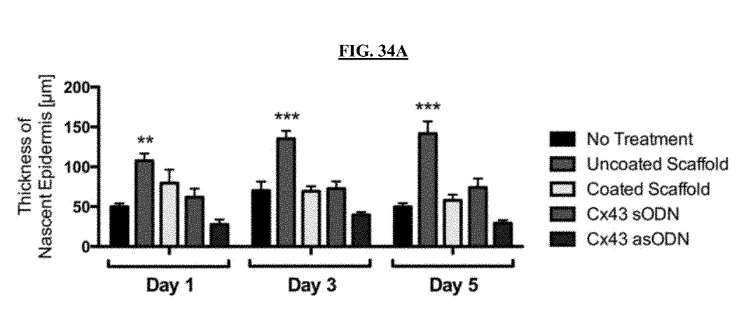

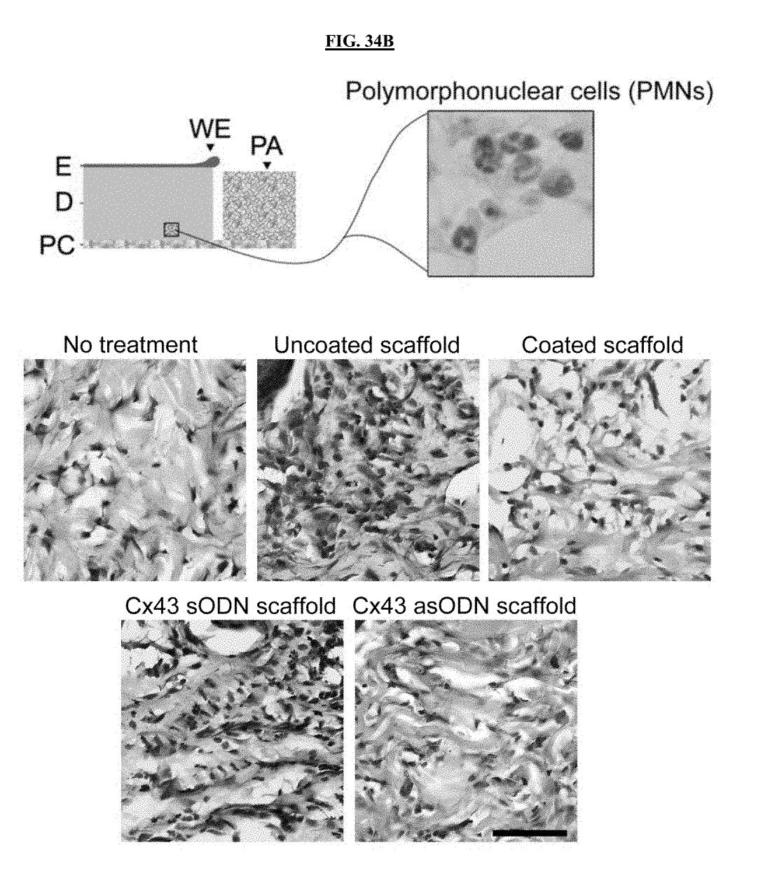

Similar measurements were also recorded at D5. At this time point a small proportion of wounds were approaching the final stages of re-epithelialisation, where the epithelial barrier is restored. Untreated wounds had re-epithelialised an average distance of 882 μm, which was only 12% higher than the averaged distance of 779 μm for all control scaffold-treated wounds (FIG. 33A). Cx43asODN coated scaffold wounds had re-epithelialised on average 1345 μm, which was a 65% increase over untreated wounds (P<0.01). The increase was again more pronounced over all the control scaffold treatments—Cx43asODN scaffold-treated wound re-epithelialisation was 70% higher than the average of the three control scaffold treatments. Scaffolds coated with polymers containing Cx43asODN resulted in significantly increased levels of wound re-epithelialisation across all the studied time points.

While wound edge Cx26 levels were greatly reduced from those at D3 across all treatments, only the application of Cx43asODN coated scaffolds resulted in a decrease in wound edge Cx26 expression from distal levels (FIGS. 33A & D). All three control scaffolds resulted in a similar increase in wound edge Cx26 expression, with an average rise of 147%. Both coated and uncoated control scaffolds resulted in higher Cx26 levels than the Cx43asODN scaffold-treated wounds.

Wounds treated with Cx43asODN at D5 maintained a downregulation of Cx43 in wound edge keratinocytes, with a 91% decrease in expression from distal levels (FIGS. 33A & C). This was lower than untreated wound edge keratinocytes, which displayed a 57% downregulation of Cx43 at the same time point. Both uncoated and coated scaffolds resulted in significantly higher levels of Cx43 expression than untreated wounds (P<0.05 and P<0.01), although significance was not achieved against the asODN coated scaffold owing to high group variability. While scaffolds appeared to have an effect on keratinocyte Cx43 levels over several days, no clear differences were observed in fibroblasts within the developing granulation tissue at this time point.

### Assessment of the Foreign Body Reaction and Inflammation

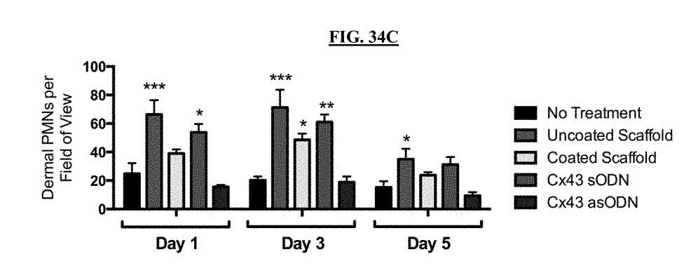

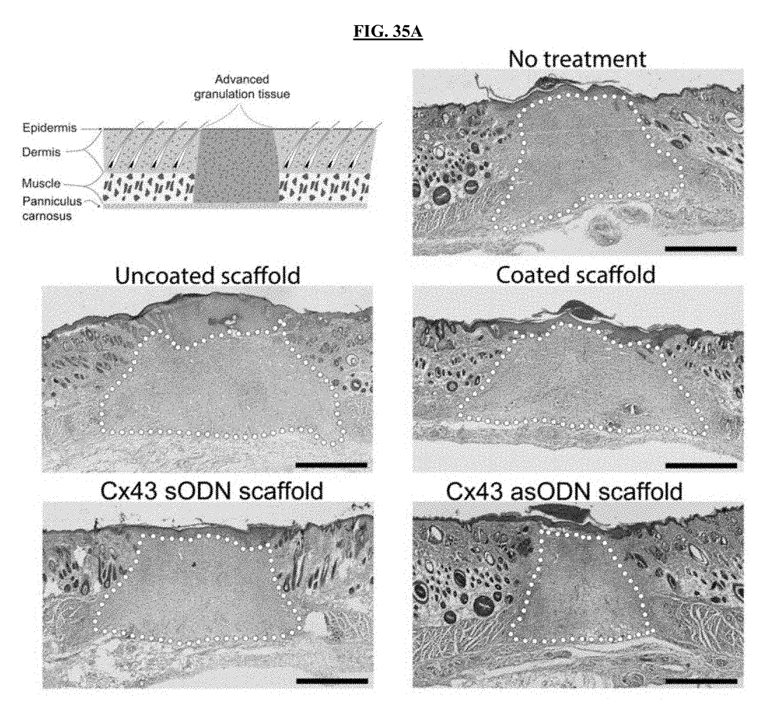

The assessment of thickening in the skin wound of the leading edge (first 150 μm) of the nascent epidermis was also measured as a component of the foreign body reaction and inflammation assessment. At D1, the average thickness of wounds treated with uncoated scaffolds was 107.5 μm (±9.1 μm), which was considerably thicker than untreated at 50 μm (FIG. 34A). Coating the scaffolds alone or with Cx43senseODN reduced the thickness slightly to 79.5 μm (±17 μm) and 61.7 μm (±11 μm), respectively. However, Cx43asODN coated scaffolds produced the thinnest epidermis at 27.8 μm (±6.2 μm). This trend continued at D3 when the untreated wounds averaged a thickness of 70.3 μm (±11.3 μm), which was similar to coated scaffolds or Cx43senseODN scaffolds but thinner than the uncoated scaffolds at 135.3 μm (±11.3 μm). The Cx43asODN coated scaffolds were still the thinnest at 39.7 μm (±3.7 μm). D5 was similar, with Cx43asODN scaffolds producing the thinnest epidermis at 29.3 μm (±3.8 μm), with untreated wounds at 49.8 μm (±4.7 μm), similar to coated scaffolds or Cx43senseODN scaffolds. Again uncoated scaffolds were the thickest at 141.7 μm (±15.2 μm).

Polymorphonuclear leukocytes (PMNs) were identified and counted in the intact dermis distal to the wound edge in order to investigate the inflammatory response to the scaffolds (FIG. 34B). At D1 untreated wounds had an average of 24.8 (±7.4) PMN cells per field of view which was reduced somewhat for Cx43asODN coated scaffolds at 15.6 (±1.3) cells (FIG. 34C). However, uncoated scaffolds generated the highest inflammatory response with 66.4 (±9.9) PMN cells, and coated and senseODN coated also causing inflammation with 39 (±2.9) and 53.8 (±5.9) PMN cells respectively. At D3, there was slightly elevated numbers of PMNs compared to D1 for all treatments. Again, counts in untreated wounds Cx43asODN coated scaffolds were similar at 20.2 (±2.7) and 18.8 (±4.1) PMN cells respectively. Uncoated scaffolds had the highest number of PMNs at 71.2 (±12.5) with coated and senseODN coated scaffolds similar at 48.6 (±4.3) and 61 (±5.3) PMNs respectively. By D5, PMN levels in distal dermis had decreased considerably across all treatments, including uncoated scaffolds. However, this was still significantly higher than untreated wounds. Cx43asODN coated scaffolds still produced the lowest PMN counts of 9.4 (±2.5) slightly lower than untreated wounds at 15.2 (±4.3). Uncoated scaffolds still had the highest inflammation with 35 (±7.3) PMN cells with coated and senseODN coated-scaffolds producing 23.8 (±2.1) and 31.2 (±5.4) PMN cells respectively, showing that inflammation was finally resolving.

### Granulation Tissue Formation Following Scaffold Application

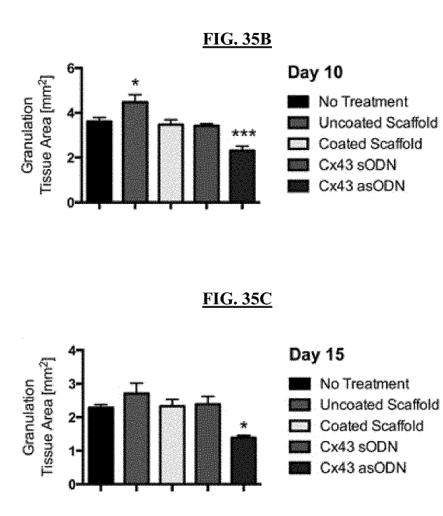

Wounds harvested at D10 and D15 were sectioned, H&E stained and the granulation tissue area measured using ImageJ (FIG. 35A). At D10 there were significant differences in the granulation tissue between the scaffold treatments (FIGS. 35A & 35B). Treatment of wounds with Cx43asODN coated scaffolds resulted in an average area of 2.30 μm², which was significantly less than untreated wounds (3.61 μm², P<0.01). The granulation tissue areas for coated and senseODN coated scaffolds were similar to untreated wounds at 3.47 μm² and 3.41 μm² respectively. However, uncoated scaffold treated wounds had a significantly larger granulation tissue area at 4.47 μm² than untreated wounds (P<0.05).

The positive effect of the bioactive, Cx43asODN coated scaffolds on wound healing continued to be seen at D15 when wounds had a granulation tissue area of 1.391 μm2 (FIG. 35C), which was significantly lower than the 2.29 μm² of untreated wounds (P<0.05). The granulation tissue area of all the other treatments were very similar with uncoated scaffold (2.70 μm²), coated scaffold (2.33 μm²) and senseODN coated scaffold (2.39 μm²).

The inventors have surprisingly shown they have fabricated a scaffold that continuously releases Cx43 asODN over 7 days. This sustained release of Cx43 asODN has (1) continued to promote re-epithelialization by downregulating Cx43 by an average 85% at the epidermal wound edge throughout the 7-day window, and (2) reduced the foreign body and inflammatory response (evaluated by PMN cell infiltration and epithelial thickening); and (3) allowed contraction and closure of the wound at a normal rate or faster than the untreated scaffold.

Controlled Release of Anti-Connexin Agent from Scaffold

To measure the amount of released asODN on the single-coated and double-coated scaffolds over time, the samples were immersed in water and the solution was measured for asODN concentration using a UV spectrophotometer using methods known in the art. FIG. 15 shows a graph of cumulative asODN release from sustained release drug delivery devices of the invention comprising (1) a scaffold which further comprises a single PCL coat, (2) a scaffold which further comprises a single PLGA coat, or (3) a scaffold which further comprises a PCL/PLGA double coat over time. Each data point represents n=3 samples, with the max and min and average values shown on the graph. The PCL/PLGA double-coated scaffold released more asODN per unit time than either of the single-coated scaffolds. Also, the PCL-coated scaffold released asODN at a slower rate than the PLGA-coated scaffold. This is likely due to the hydrolysis of the PCL proceeding at a slower rate than the hydrolysis of the PLGA. The double-coated scaffold exhibits both an initial release of the asODN from the PLGA outer layer, followed by a slower release of the asODN, presumably from the release of the asODN from the PCL inner layer. FIG. 16B shows the results of a repeat of this experiment, demonstrating the reproducibility of the relative release rates for the various coatings and mixture of coatings.

To demonstrate a greater difference in the relative release rates, and to create coated scaffolds comprising higher amounts of an anti-connexin agent, coated scaffolds with multiple layers of coatings were made and their asODN release rates analyzed. FIG. 16C shows the release rates (cumulative amounts released at each timepoint) of asODN-polymer coated scaffolds comprising four layers of each coating. The scaffolds were subjected to four dip processes (as described above) for the PCL, PLGA, and the PCL-PLGA double-coat. The 4-layer PLGA-coated scaffold released asODN very rapidly initially, then reached saturation at about 2 days. The 4-layer PCL-coated scaffold released asODN more slowly than the PLGA-coated scaffold, and did not reach saturation after 7 days of measurement. The 4-layer dual-coated PCL-PLGA coated scaffold exhibited an initial rapid release of the asODN, followed by a slower release of asODN.

The difference between the cumulative amount of asODN released at any given timepoint and the prior timepoint from each of the 4-layer sustained release drug delivery devices was measured as the “new asODN elution” and shown in FIG. 16D. The new asODN elution amounts for the 4-layer PLGA-coated scaffold show that the asODN is released very rapidly at about 2 days, and after about 4 days no further asODN is released from the PLGA-coated scaffold. The 4-layer PCL-coated scaffold is released quite moderately from about 1 to 4 days, after which the amount of released asODN drops, but continues to release asODN until about 7 days. The 4-layer PCL-PLGA dual-coated scaffold exhibits an initial rapid release of asODN at the first and second day, and then continues to release asODN up until about 7 days.

Scaffolds coated in four layers of PCL, produced a more sustained release profile in which asODN elution only began to plateau at around 6-7 days. While coating scaffolds with 4 layers of PCL resulted in a sustained release of asODN, coating them with an additional 4 layers of PLGA (“double-coated”) achieved the greatest overall release of Cx43asODN across the 7-day assessment. Additionally, the elution profile for double-coated scaffolds appeared to adopt the characteristics of both the individual PLGA and PCL coatings. There was a large burst release of asODN in the first 24 h characteristic of PLGA as well as the more sustained release profile of PCL throughout the 7-day assessment. This includes a small peak of elution around 3-4 days, which was also present in the PCL-only coated scaffold, and could be explained by hydrolysis of the scaffold coating (Tarvainen, T., et al., (2002), Eur J Pharm Sci 16, 323-31). Second peaks of elution have also been reported using other, different multi-layered approaches, such as two-layered scaffold fibres containing an inner layer of PLGA mixed with gentamicin, lidocaine and vancomycin, and a blank outer layer (Chen, D. et al., (2012) Int J Nanomedicine 7, 763-71). The occurrence of a second peak appears to be linked to the use of multiple layers, and may be the result of sufficient hydrolysis of outer layers to expose the inner layers. In another study, stents were coated with an inside polymer layer containing Sirolimus as well as an outer layer containing no drug (Schofer, J., et al. (2003), Lancet 362, 1093-9). The group reported that the use of an outer coating provided a barrier slowed the elution of drug from the inner layer and that 80% of the drug eluted over a 30-day period following implantation. The initial burst release of asODN observed could be useful in a wound setting as Cx43 must be downregulated to attenuate inflammation and for the re-epithelialisation process to initiate (Mori, R. et al., (2006) J Cell Sci 119, 5193-203).

The finding that multiple coating layers improved the overall duration of asODN elution is also in agreement with the results of a clinical trial study that used multi-layered stent coatings with other drugs. In this study, stents were coated with an inside polymer layer containing Sirolimus as well as an outer layer containing no drug (Schofer, J., et al., (2003), Lancet 362, 1093-9). The group found that the use of an outer coating provided a barrier, which slowed the elution of drug from the inner layer so that 80% of the drug eluted over a 30-day period following implantation.

The discovery of a combined PLGA and PCL coating resulted in an average asODN elution of 265 μg within 7 days in a sustained manner. In order to test whether scaffolds were similarly effective in vivo they were applied to 6 mm full-thickness wounds, this time made to the dorsum of rats. Macroscopically, scaffolds coated in the synthetic polymers appeared to integrate poorly with the wound edge, a feature possibly due to the coating layers producing a more rigid scaffold. Scaffolds were loosely associated with wounds, and during processing to permit histological analysis it was not uncommon for scaffolds to separate from wounds. However, at D3 and D5 the Cx43asODN coated scaffold-treated wounds appeared to reduce in size and shrink in the wound compared to the uncoated scaffold-treated wounds. This could be due to the fact that the coating method used did not have cross-linking of scaffolds, thereby permitting the collagenous fibres in the scaffold to break down more rapidly in conjunction with wound contraction. Scaffolds that had been coated without Cx43asODN or coated with sense ODN at D3 and D5 appeared to prevent wound closure to a small degree, relative to untreated wounds. The smallest wounds were those treated with Cx43asODN.

Scaffold Protection of Anti-Connexin Agent from Serum Nucleases

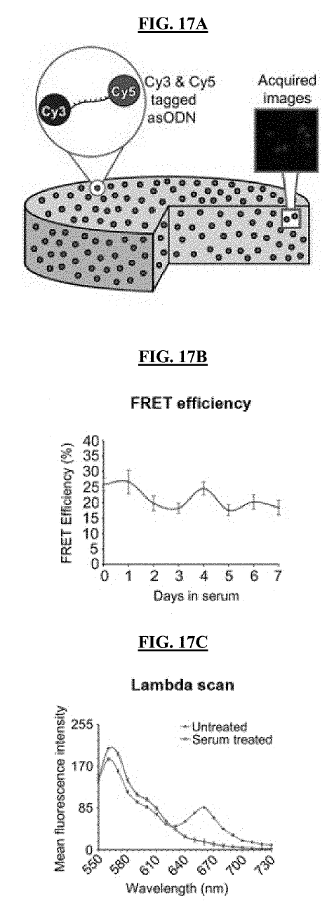

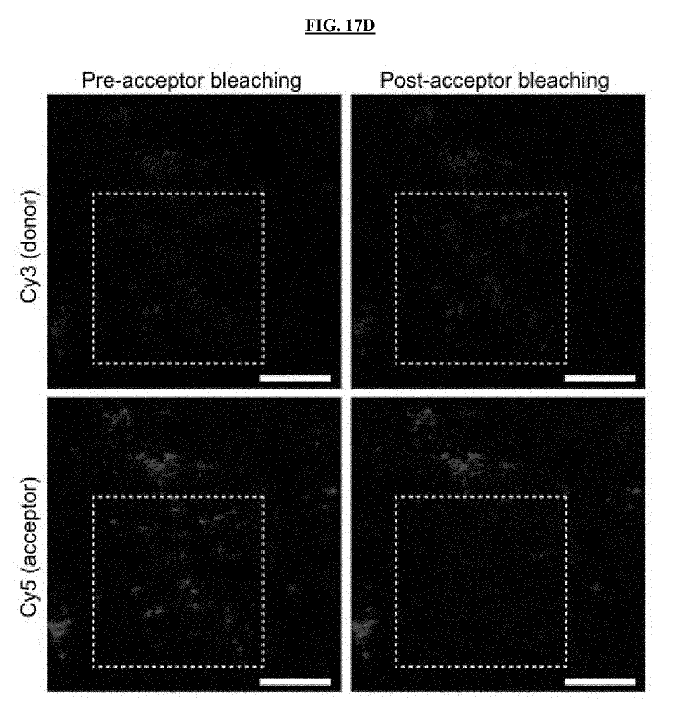

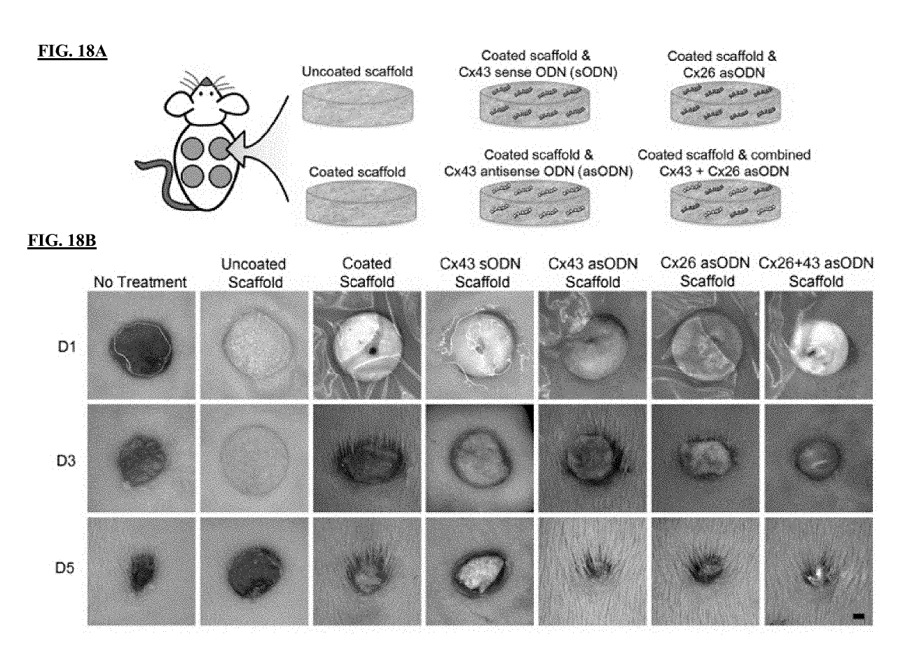

In order to evaluate the integrity of asODN incorporated into the polymer coatings, Förster resonance energy transfer (FRET) imaging was used. Collagen scaffolds were generated that were loaded with asODNs labelled with the dye Cy5 at the 3′ terminus and the Cy3 dye at the 5′ terminus which are capable of FRET when intact (FIG. 17A). Immediately after coating scaffolds, FRET efficiency was calculated to be 25%, (FIGS. 17B & 17D) which previous studies have accepted as genuine (Lai, X. J., et al. (2014), Cell Death Dis 5, el 184). In order to assess whether asODN contained within the coatings was afforded a degree of protection against nuclease activity present in wounds, a series of FRET asODN coated scaffolds were submerged in foetal bovine serum (FBS) at 37° C. for 7 days. Scaffolds were harvested every 24 h and FRET efficiencies were determined for 6 individual asODN clusters per time point (FIG. 17B). The average FRET efficiencies calculated between D1 and D7 ranged from 17.5%-26.7%, in all cases remaining above 15% (FIG. 17B).

In order to confirm that FRET was not occurring due to inadequate nuclease activity of the FBS used, FRET labelled Cx43 asODN was mixed with FBS for 6 h at 37° C. A lambda scan was then performed on the FRET-FBS solution on a Leica confocal microscope, using a 533 nm laser to stimulate CY3. A second lambda scan was performed on a control solution of FRET labelled asODN that had been mixed with water and the two scan outputs were compared. The resultant scans showed a second peak of emission between 650-680 nm in the control asODN solution. This this peak was absent in the solution treated with FBS indicating that the asODN had been broken down (FIG. 17C).

To demonstrate that the polymer coating on the scaffold can protect the asODN from serum nucleases, the stability of model oligonucleotides was measured after exposure to fetal bovine serum at various timepoints. The sequence of the Cx43 asODN, listed above, was obtained from Sigma Genosis with a 3′ Cy3 label and a 5′ Cy5 label to create a Fluorescence Resonance Energy Transfer (“FRET”) pair on the intact oligonucleotide. This model oligonucleotide was used in place of the asODN to create coated-scaffolds in the methods described above, FIG. 17A. Coated scaffolds were immersed in 37 C fetal bovine serum (FBS, Sigma Aldrich PN 12003C) for up to 7 days (timepoints 0 h, D1, D2, D3, D4, D5, D6, and D7). FRET is a confocal microscopy technique that can be used to illustrate the presence of intact asODN. It involves the bleaching of one fluorophore (Cy5, acceptor) attached to an asODN sequence in order to detect increased levels of the proximally attached second fluorophore (Cy3, donor). FRET-capable asODN was detected in the scaffold coating at all timepoints as indicated by software outputted FRET efficiency percentages (FIG. 17B). FRET efficiencies were an indicator of successful FRET occurring and these were all above 15%—a level which indicated that asODN was intact and that the scaffold provided a level of protection against nucleases present in the FBS. N=6 regions of interest were used to calculate an averaged FRET efficiency at each timepoint in the software. A lambda scan (FIG. 17C) was also performed to show that asODN treated with serum (red line) were no longer producing a characteristic ‘FRET’ second peak of emission like untreated asODN (green line); This served as a positive control that the serum used was effective at destroying naked asODN and the results in this figure were not due to the serum being ineffective as a nuclease. FIG. 17D shows a typical bleaching of the acceptor, followed by an increase in intensity of the donor. Thus, the coating provided a level of protection to asODN against serum nucleases as illustrated by FRET imaging techniques.

While spectrophotometry-based elution profiling showed that a considerable quantity of asODN eluted from PCL-PLGA double-coated scaffolds over 7 days, it did not reveal whether the homogenisation and solvent processing steps performed in the coating process led to any damage to the structure of the incorporated DNA. The FRET acceptor photobleaching was performed on sectioned scaffolds coated using a Cy3 and Cy5 dual labelled Cx43asODN sequence. This technique has been successfully used to study asODN integrity and is favoured for both its accuracy and simplicity (Cronin, M. et al, (2006), Front Biosci 11, 2967-75; Rupenthal, L, et al., (2012) Current Pharmaceutical Analysis 8, 20-2). Immediately after coating (0 h) there was a FRET efficiency of 25.9%. A minimum efficiency of 15-20% has been previously used as an indicator that the FRET occurring was genuine, while lower percentages may reflect background variation (Lai, X., et al., (2014) Cell Death Dis 5, el 184). A FRET efficiency of 25.9% obtained for the double-coated scaffold can be interpreted to mean that the coating contained intact asODN, confirming that the coating process did not render the incorporated asODN non-functional. In order to test whether the multiple coatings afforded a level of protection against DNA degradation, FRET-labelled scaffolds were submerged in fetal bovine serum for up to 7 days and assessed each day to expose them to the DNAase activity of the serum. This revealed similarly high FRET efficiencies within the inner coating layers of scaffolds, even after several days in serum containing nuclease enzymes. The ability of serum enzymes to degrade DNA was also confirmed through a series of lambda scans which showed that serum treated asODN did not produce the same FRET peak, which suggests that the FBS used was effective in degrading free asODN.

In Vivo Treatment with Scaffold Sustained Release Drug Delivery Devices—Single Anti-Connexin Agent Coated Scaffolds Vs. Plurality of Anti-Connexin Agent Coated Scaffolds

As described herein, the inventors have made the surprising discovery that a scaffold comprising multiple anti-connexin agents has a synergistic effect on wound healing. The anti-connexin agents used are two anti-sense anticonnexin oligonucleotides, but could also be an anti-sense anticonnexin oligonucleotide and another anti-connexin agent which is not an oligonucleotide. For example, the other anti-connexin agent which is not an oligonucleotide could be a small molecule, for example tonabersat, or an anti-connexin peptide, for example peptide 5 (directed to connexin 43), or any other peptide directed to a connexin protein or protein fragment.

Elevated Cx26 has also been found to delay restoration of the epidermal barrier and promote the formation of an inflammatory response (Djalilian, A. et al, (2006) J Clin Invest 116, 1243-53). Cx26 was found to be slightly elevated in wounds containing uncoated scaffolds but was significantly reduced in Cx43asODN coated scaffolds on days 3 & 5. The reduction in Cx26 levels seen in the Cx43asODN coated scaffolds may reflect a reduction in the inflammatory response in these wounds, which was seen as a reduction in the number of neutrophils in the wound edge dermis and a reduced thickness of the nascent epidermis.

The methods used for histology analysis were: (1) standard tissue cyrosectioning, (2) Hematoxylin and eosin staining (“H&E”), Cx26 staining (using Rabbit anti-Cx26 primary, Millipore PN AB 8143, diluted 1:200; and Goat anti-rabbit Alexa 488 secondary, Life Technologies PN A11034, diluted 1:400, (3) confocal microscopy (single optical sectio image acquisition), and (4) ImageJ image analysis of the microscope images. The relative areas of the H/E stains were analyzed using ImageJ area finder tool. The method used for detecting the concentration of asODN released from a scaffold was the measurement over time using a UV spectrophotometer using methods known in the art.

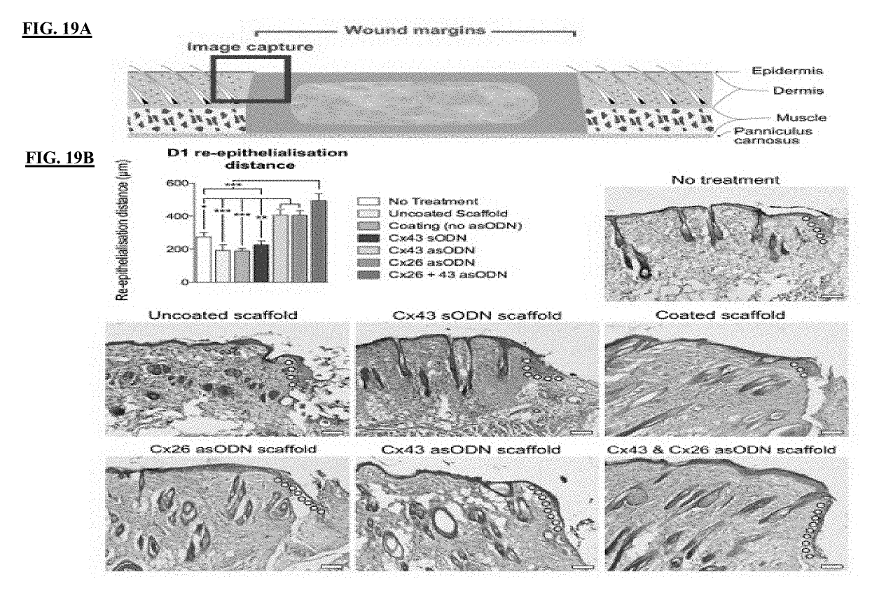

Full-thickness 6 mm wounds were made to the backs of rats. Scaffolds were applied to full-thickness back wounds in 6-week Sprague Dawley rats (4 wounds per animal, as shown in FIGS. 18A and 18B). Cull time-points were Day 1 (n=8), Day 3 (n=8), Day 5 (n=8), Day 10 (=7) and Day 15 (n=7).

There were 7 treatment groups, consisting of: (1) No treatment, (2) Uncoated scaffolds, (3) Coated scaffolds only (PCL and PLGA polymers only, no asODN), (4) Coated scaffold comprising Cx43 sODN (with PCL and PLGA), (5) Coated scaffolds comprising Cx43 asODN (with PCL and PLGA), (6) Coated scaffolds with and Cx26 asODN Sequence 1 and Cx26asODN sequence 2 (with PCL and PLGA), and (7) Coated both Cx26 asODN Sequence 1, Cx26asODN sequence 2, and Cx43asODN (with PCL and PLGA).

A visual analysis of the wound sites before and during healing for each of the treatment groups is shown in FIG. 18B. The uncoated-scaffold exhibited a negative treatment effect, because the scaffold prevented the wound from closure. The scaffold coated with Cx43 sODN (the “sense” oligonucleotide, which upregulates the production of Cx43) showed worse healing than the no treatment wound. However, the scaffold coated with Cx43 asODN (“antisense”) showed remarkable improvement in healing compared to both the upregulated Cx43 sODN treatment and the no treatment wound. While the wound treated with Cx26 asODN healed about the same as the wound treated with Cx43 asODN, the wound treated with both Cx26 asODN and Cx43 asODN showed surprisingly remarkable improvement in wound healing over either of the single asODN treatments. This demonstrates that the wound area can be significantly reduced by treatment with Cx43 asODN, Cx26 asODN, and especially with the combination of the two oligonucleotides. This also demonstrates that preventing Cx43 upregulation with delivery of Cx43 asODN to the wound improves healing of the wound.

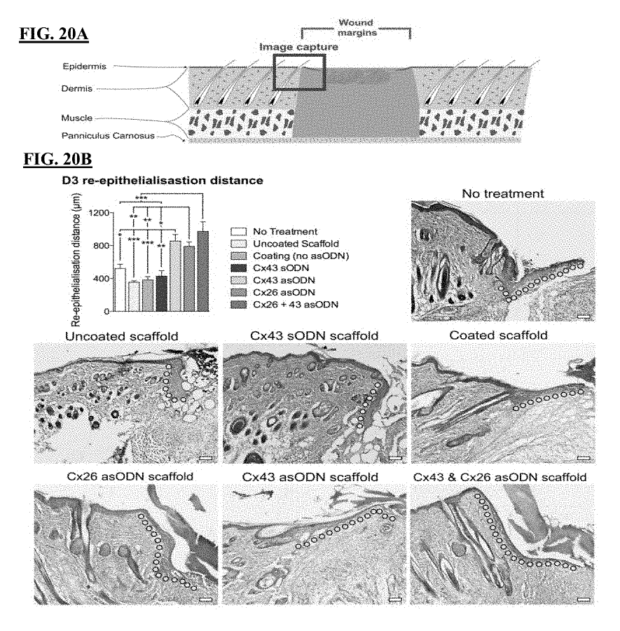

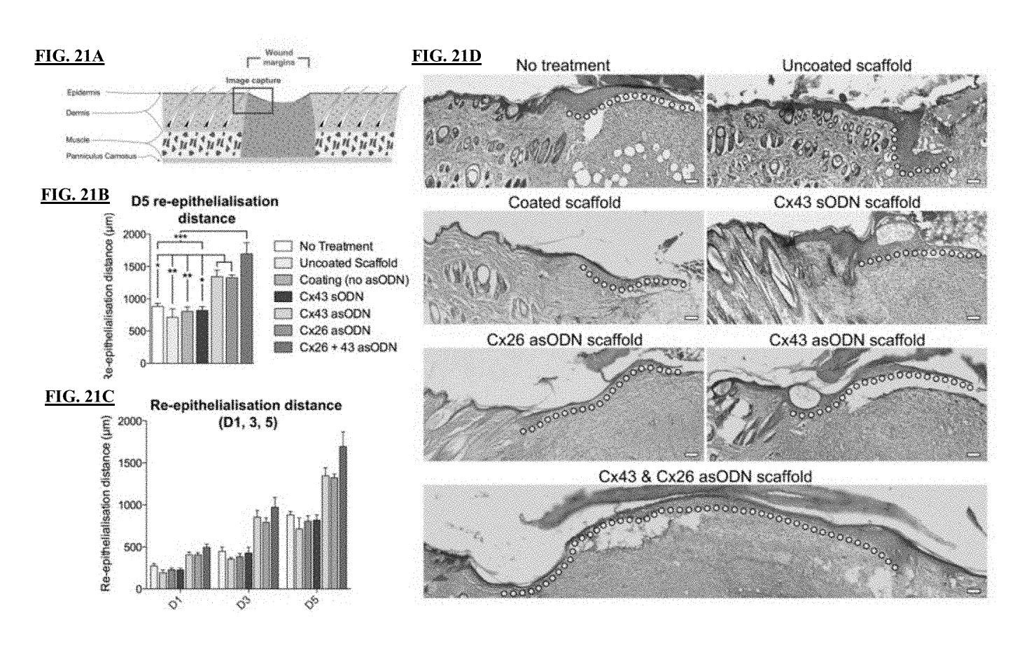

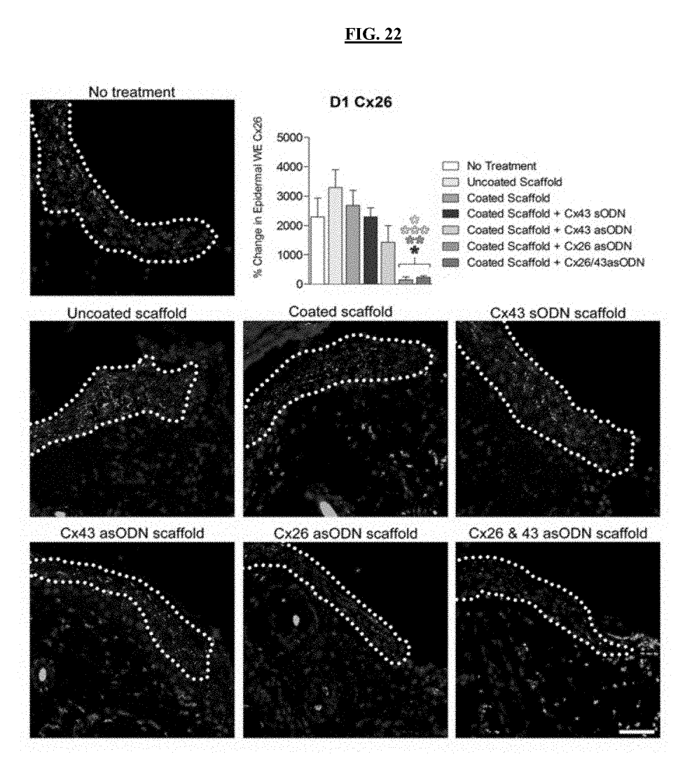

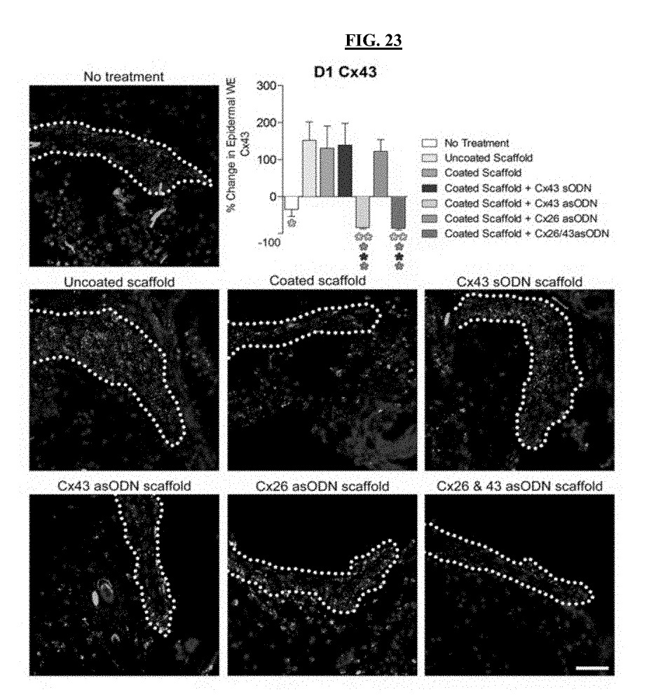

Re-epithelialization of the wound was monitored by histological imaging of a side profile of the wound edge. As shown in FIGS. 19A and 19B, an image of the wound edge was captured at Day 1 to measure the relative degree of wound healing for the various treatment groups listed above. Images of the wound edges of the treatment groups at Day 3 are shown in FIGS. 20A and 20B. Images of the wound edges of the treatment groups at Day 5 are shown in FIGS. 21A, 21B, 21C, and 21D. The re-epithelialization distance was longer for the asODN-coated scaffolds than for the scaffolds not coated with asODN. The re-epithelialization distance was longest for the scaffold coated with both the Cx26 asODNs and the Cx43 asODN, indicating a synergistic effect on the wound healing by having all asODNs present. Re-epithelialization is improved with Cx26asODN coated scaffolds relative to control treatments (D1/D3/D5) relative to controls. Cx26 asODN can induce a 2-3 fold increase in nascent epithelium growth in the first 5 days of healing.

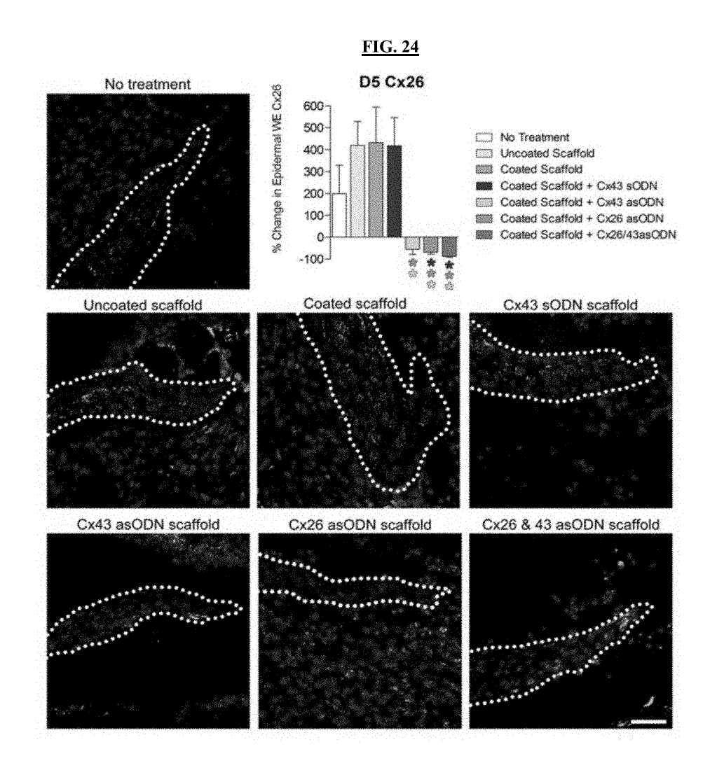

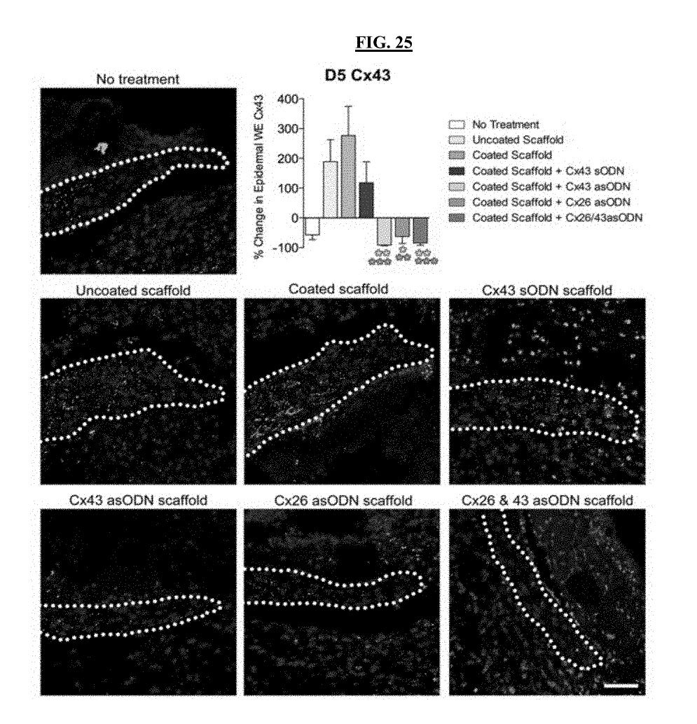

Selective staining of the wound edge of the various treatment groups shows the localization of the Cx26 asODNs and Cx43 asODN within the wound after release from the scaffold. FIG. 22 shows the selective H&E staining of Cx26 which shows the selective localization of Cx26 at wounds at Day 1 of healing which were not treated with Cx26 asODN. The no treatment, uncoated scaffold, Cx43 asODN coated scaffold, and Cx43 sODN coated scaffold treatment groups exhibited a large increase of Cx26 relative to the Cx26 asODN and Cx26 asODN+Cx43 asODN coated-scaffold treatment groups. This indicates that preventing the upregulation of Cx26 is at least one mechanism of the improved wound healing observed in the visual images of FIGS. 18A and 18B. FIG. 23 shows the selective staining of Cx43, which shows the selective localization of Cx43 at wounds at Day 1 of healing which were not treated with Cx43 asODN. The no treatment, uncoated scaffold, Cx26 asODN coated scaffold, and Cx43 sODN coated scaffold treatment groups exhibited a large increase of Cx43 relative to the Cx43 asODN and Cx26 asODN+Cx43 asODN coated-scaffold treatment groups. This indicates that preventing the upregulation of Cx43 is at least one mechanism of the improved wound healing observed in the visual images of FIGS. 18A and 18B. The effects are repeated at the Day 5 timepoint as shown in FIGS. 24 and 25 for the selective stain images for Cx26 and Cx43 respectively. The Cx26 and Cx43 are not upregulated in the treatment groups which exhibited the better wound healing. Thus, the prevention of the upregulation of Cx26 and Cx43 via the anti-connexin agent is the mechanism by which the improved wound healing is observed.

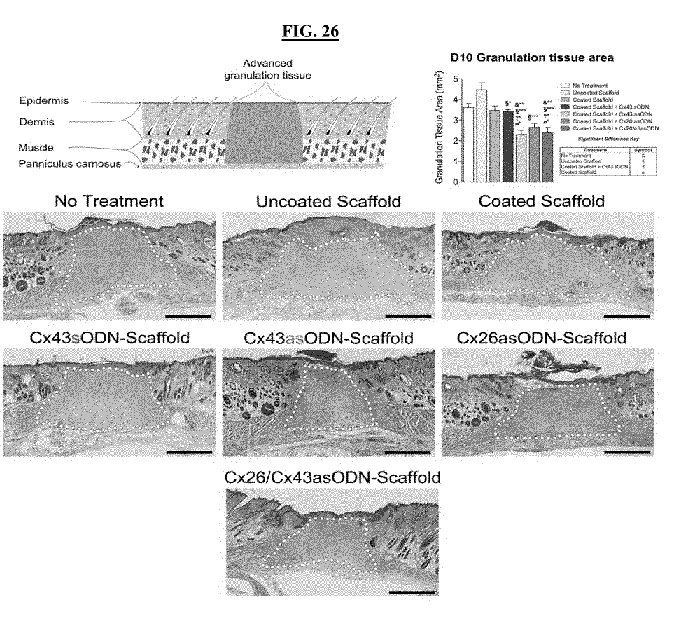

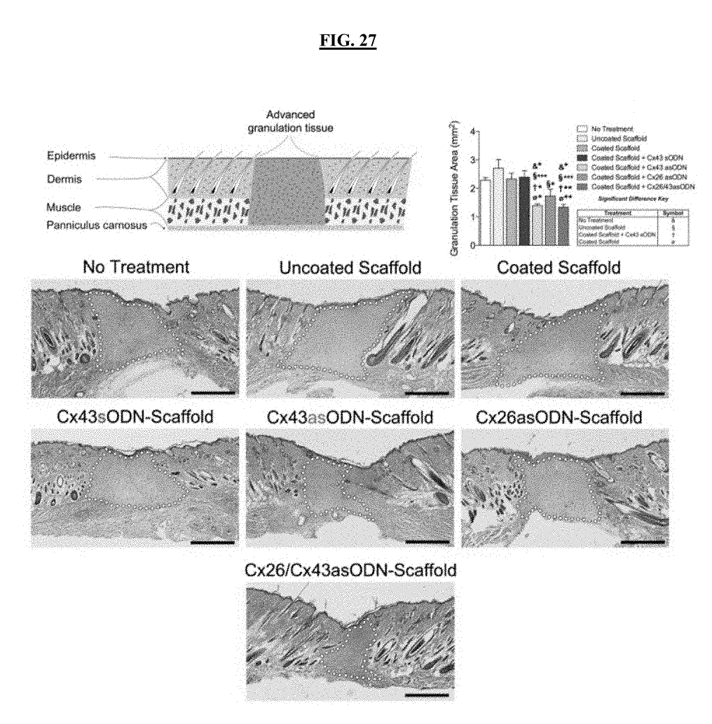

Reduction in granulation tissue area is a feature of wound healing resolution. A more rapid resolution generally indicates a more rapid overall healing. Granulation tissue area was measured for the histological samples of the various treatment groups at Day 10 (FIG. 26) and Day 15 (FIG. 27) of healing. The granulation tissue area (the area outlined by white dots in the images in FIGS. 26 and 27) is reduced at Day 10 and Day 15 with Cx26 asODN only scaffolds relative to controls. Furthermore, the Cx43 asODN coated scaffolds and the combined Cx26 asODN+Cx43 asODN coated scaffolds exhibit significant reductions in the granulation tissue area compared to controls. This indicates that the presence of the Cx26 asODN and Cx43 asODN is associated with a significant amount of wound healing.

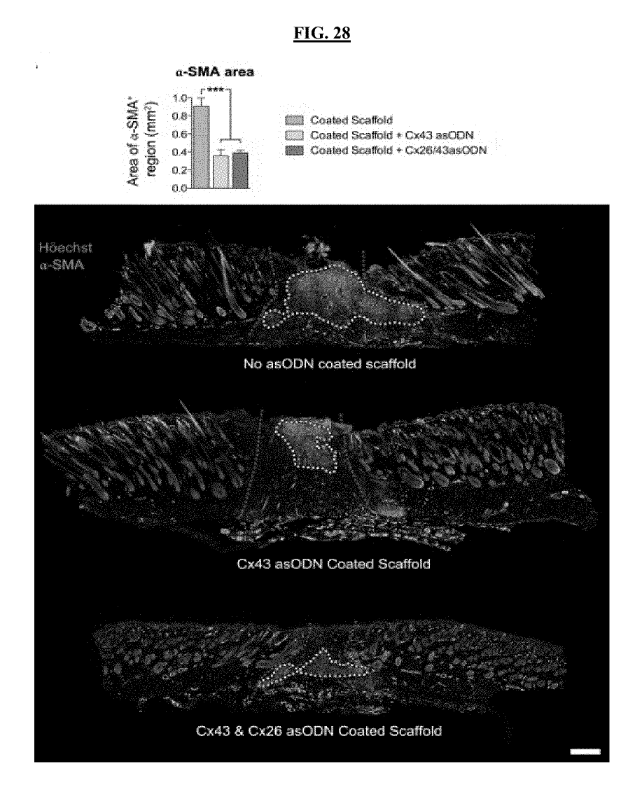

As shown in FIG. 28, the selective staining of alpha-smooth muscle actin in the treatment groups at Day 10 after healing indicates that wound area is reduced after treatment of the scaffold-coated Cx43 asODN and the Cx43 asODN+Cx26 asODN dual-coated scaffold, relative to controls.

The net effect of scaffolds coated with both Cx26asODN and Cx43asODN demonstrated that the combination was better than scaffolds treated with either asODN alone.

The presence of hair regrowth, scab formation and the ability of scaffolds to mask the wound bed prevented accurate measurements being made from macroscopic evaluations, so the wounds were examined microscopically. Coated scaffolds appeared to act purely as a drug-delivery device while offering little to no structural support to the wound. However, coating scaffolds with Cx43asODN resulted in an almost doubling of wound re-epithelialisation distance at each time point examined compared to scaffolds coated with the sense sequence. Furthermore, the Cx43asODN scaffold-treated wounds had re-epithelialised about 40% further than untreated wounds (P<0.01). This indicates that coating non-cross-linked scaffolds with Cx43asODN actually improved wound healing, unlike previous studies where bioactivation of cross-linked collagen scaffolds by dipping them in Cx43asODN Pluronic gel appeared to only reduce the negative foreign body side effects of using cross-linked collagen scaffolds (Gilmartin et al., 2013). The inventor's recognized newly identified mechanism of action for treating wounds is supported by previous studies, where wound re-epithelialisation was found to be enhanced following Cx43asODN application. This is the first time a similar result has been achieved using a sustained Cx43asODN delivery approach rather than a bolus of drug at the start of the healing process.

The Cx43asODN significantly reduced Cx43 expression in wound edge keratinocytes at D1, D3 and D5, relative to the control scaffolds, which is likely to enhance their rate of migration. The finding that Cx43 remained downregulated even at the later time point of D5 indicated that the coating continued to elute Cx43asODN, since the control coated scaffolds had a significantly higher Cx43 expression in wound edge keratinocytes. In our previous study where we bioactivated collagen scaffolds by soaking them in Cx43asODN Pluronic gel, the antisense had long been destroyed by D5 and Cx43 levels were elevated (Gilmartin et al., 2013).

While in vitro assays highlighted the ability of scaffold coatings to elute asODN in a sustained manner, evaluation of treated wounds at D10 and 15 gave a clearer indication of the long-term biological efficacy of the coatings. At these later time points scaffolds were no longer visible within wounds when they were harvested. This could be due to degradation of the scaffold fibres or the loss of the shrunken scaffolds, which were never integrated into the wound site (Ishaug, S. et al., (1994), J Biomed Mater Res 28, 1445-53). The loss of the Tegaderm film securing scaffolds before these time points also adds to this possibility. Wounds that received a Cx43asODN coated scaffold had significantly smaller regions of granulation tissue, the tissue rich in fibroblasts and blood vessels, when compared to all the other treatments. These wounds appeared to be further along in the wound maturation process than the other treatments.

These new coated scaffolds that provide a sustained release of Cx43asODN not only overcome the foreign body reaction caused by scaffolds but also significantly enhance the healing process. These could be particularly effective in treating chronic wounds that significantly overexpress Cx43 (Sutcliffe, J. E., et al., 2015, Br J Dermatol.). This may be particularly applicable to venous leg ulcers where the standard of care is to apply compression bandages that are changed on a weekly basis. The slow release would enable sustained drug delivery for up to one week with one dosing.

### Example 8

### Diabetic Wound Healing Efficacy

Anti-connexin 26 (Cx26) polynucleotide preparations, alone or combination with other agents, including other anti-connexin agents (e.g., an anti-Cx43 polynucleotide, peptide, or peptidomimetic) are evaluated for the efficacy in wound healing in rat diabetic model. Diabetes is induced in adult Sprague-Dawley rats (350-400 g) by a single intraperitoneal injection containing streptozotocin, 65 mg/kg, in citrate buffer (Shotton H R, Clarke S, Lincoln J. Diabetes (52); 157-164, (2003)). (N=six diabetic, six control per time point). The effectiveness of treatments of diabetic autonomic neuropathy is not the same in autonomic nerves supplying different organs (Id.). Most diabetic wound-healing studies are carried out two weeks after diabetes induction and the same time point is used for this wound healing study. However, connexin expression in diabetic rat skin is also examined at eight weeks (n=6 diabetic, 6 control per time point) to confirm that the changes detected at two weeks will remain the same. Normal back skin is excised, cryosectioned, immunostained for connexins, imaged by confocal microscopy and the staining quantified as described in Saitongdee, et al. (2000), Cardiovascular Res. 47, 108-115.

Rats are anaesthetised with halothane and their backs are shaved. Two pairs of 5×5 mm full thickness excision wounds are made. 100-500 micrograms of anti-connexin 26 peptide comprising SRPTEKT (SEQ ID NO:38), and/or SRPTEKTVFTV (SEQ ID NO:37) in Pluronic F-127 gel is applied to one wound and control (Pluronic F-127 gel only) applied to the second wound. 10 μM of the anti-Cx26 polynucleotide in Pluronic F-127 gel is applied to one wound and control (sense) gel to the other at either within 1 minute, 5 minutes, 10 minutes, 30 minutes, 1 hour, or 6 hours.

Tissue is harvested on days 1, 2, 5, 10, and 15 after wounding, and sectioned in preparation for connexin immunohistochemistry or H&E staining (Coutinho, et al. (2003), Cell Biol. Int. 27:525-541). N=six diabetic, six control rats per time point.

Intercellular communication is assessed by applying a 4% solution of Lucifer Yellow CH (Sigma) in a pledget of gelfoam into a fresh, full thickness skin incision. Dye is allowed to transfer for 5 minutes prior to removal of the gel foam and fixation of the tissue. A 10 kD Kd FITC-dextran that will enter injured cells but not pass through gap junctions is used as a control. Tissues are cryosectioned and imaged by confocal microscopy on a Leica SP2UV (Leica, Milton Keynes, UK). Transferred dyes and connexin immunostaining are examined using a confocal microscope. Optimal gain and offset are set in advance and kept constant during the image acquisition process. A series of single optical section images are taken to generate a montage of the skin from the cut. Digital images (eight bit) are analysed using Image-J software (NIH).

To assess dye transfer, a 1500×30 pixel region-of-interest box is placed from the cut edge in the mid-dermis and an image intensity graph across the box is generated. A grey level intensity drop below 50 is taken as the point where Lucifer Yellow has traveled. Similarly, in the epidermis, the distance from the cut to where the Lucifer Yellow signal dropped below 50 is recorded.

A minimum of three images are analyzed from each animal. To compare levels of connexin protein, six single optical section images of dermis or epidermis are taken from different sections for each wound. All parameters of laser power, pinhole, gain/offset and objective are kept constant across both control and diabetic groups. Connexin expression is quantified as described in Saitongdee, et al. (2000), supra. A threshold is set to detect gap-junction plaques with minimal background noise and is then kept constant for all images. The number and size of connexin plaques are recorded for each image and expressed per 100 μm of epidermis or 10000 μm² of dermis. This approach has proved to be much more accurate than Western blot as it generates information on protein expression at the cellular level. Western blots are unable to distinguish between epidermal and dermal cells or detect effects of proximity to the wound edge.

Using this approach, connexin levels in keratinocytes in a zone at the wound edge (WE) and in a zone 500 μm away (AD) are able to be quantified either one or two days after wounding. At day five after wounding, an additional zone of the leading edge (LE) of the nascent epidermis is also imaged. Images of H&E staining are taken using a Leica DMLFS microscope with a DC300F digital camera. Measurements for re-epithelialization rate are described in detail in Qiu, et al. (2003), Curr. Biol. 13:1697-1703. All numerical differences between treatments are tested for significance using the Wilcoxon matched-pairs signed-ranks test as implemented in Statview 5.0.1.

Relative connexin staining levels in normal and STZ diabetic rat skin at two weeks and eight weeks after induction of diabetes are measured and compared. Graphs are plotted to show the numbers of plaques in the epidermis and dermis. Images of typical connexin 43 and connexin 26 immunostaining in control and diabetic skin at eight weeks are acquired (arrowheads mark the boundary between the epidermis and dermis; scale bar 25 μm). The relative distances that the gap-junction-permeant dye, Lucifer Yellow, traveled in five minutes in the epidermis and dermis of the control and diabetic rats are quantified.

Typically, punctate connexin 43 immunostaining is found in the basal layer of the epidermis, and in dermal fibroblasts, hair follicles, blood vessels and appendages. However, in diabetic skin, connexin 43 staining may be significantly reduced in the epidermis, in terms of both size and number of gap junction plaques. Staining for connexin 26 in the upper layers of the epidermis may be similarly reduced in diabetic epidermis.

To assess cell-cell communication in diabetic epidermis and dermis, the extent of transfer of the gap-junction-permeant dye Lucifer Yellow through the tissue in five minutes is examined. Elevated expression of connexin 43 protein and increased communication has been reported in human diabetic fibroblasts (Abdullah, et al. (1999), Endocrine 10:35-41); and mixed responses of different connexins to diabetes have been noted in the renal system (Zhang and Hill (2005), Kidney Int. 68:1171-1185).

Relative rates of re-epithelialization and responses of connexin 26 protein levels following injury in control and diabetic epidermis are measured. Staining is quantified by counting plaques at one and two days after wounding in epidermis at the wound edge (WE) and adjacent (AD) epidermis, 500 μm away. On day five, an additional zone at the leading edge (LE) of the nascent epidermis is quantified.

Connexin 26 staining (green) and nuclear staining (blue) at the epidermal wound edge of control and diabetic skin during the wound-healing process are measured and the processed by image analysis.

To determine the dynamic responses of connexin expression to injury, connexin staining in keratinocytes at the wound edge (WE) and in an adjacent zone 500 μm away (AD) is quantified at one and two days after wounding. At after five days the leading edge (LE) keratinocytes is imaged.

The effect of the possible increase of connexin 26 protein in diabetic WE keratinocytes is assessed by preventing the increase with a connexin 26-specific antisense gel, applied to the wound at the time of injury.

A finding of abnormal upregulation of connexin 26 in the epidermal wound edge in diabetes is significant, and has the potential to affect the process of wound closure in different ways. The formation of communication compartments within the regenerating epidermis has been proposed to play a role in wound healing (Martin (1997), Science 276:75-81; Lampe, et al. (1998), J. Cell Biol. 143:1735-1747; Hodgins (2004), J. Invest. Dermatol. 122: commentary). Delay in wound healing in diabetes could reflect the additional time required for connexin 26 expression to downregulate to a point where such a compartmentalization can occur.

### Example 9

### Diabetic Wound Healing Improvement

Wound healing efficacy in a diabetic subject is investigated after sequentially administering an anti-connexin 26 peptide preparation followed by administration of anti-connexin 26 polynucleotide preparation prepared in vivo to diabetic male Sprague Dawley rats. In order to quantify the wound healing in a diabetic subject, the tensile strength of the wounds is investigated, with a higher tensile strength reflecting an improvement in wound healing.

The diabetic rat animal model is an established model system for investigating diabetes-associated wounds, which heal poorly (Davidson, Arch. Dermatol. Res. 290: S1-S11). Since diabetes is accompanied by microangiopathy, this animal model is also suitable for investigating arterially determined disturbances in wound healing.

In order to induce the diabetes, rats having a bodyweight of 250-300 g are injected i.p. with a freshly prepared aqueous solution of streptozotocin (Sigma) (50 mg/kg of bodyweight). The blood sugar of the animals is checked 7-9 days after induction, with a blood sugar level value of more than 200 mg/dL confirming the diabetic state. The diabetic rats and the nondiabetic control animals are subsequently anaesthetized with a mixture consisting of 2% O₂ (2 l/min) and 1.25% isoflurane. The back is depilated and 2 sites are marked on the back of each animal for subsequent wounding. Incision wounds of 1 cm in length are then made through the wound sites and the wounds are closed with wound clips.

100-500 micrograms of anti-connexin 26 peptide in Pluronic F-127 gel (Cx26 asODN Antisense Sequence 1 or Cx26 asODN Antisense Sequence 2) is applied to one wound and control (Pluronic F-127 gel only) applied to the second wound. Thereafter, 10 μM of an anti-Cx26 oligodeoxynucleotide in Pluronic F-127 gel is applied one wound and control (sense) gel to the other at either within 1 minute, 5 minutes, 10 minutes, 30 minutes, 1 hour, or 6 hours. When a peptide is used, the peptide may comprise SRPTEKT (SEQ ID NO:38), e.g., SRPTEKTVFTV (SEQ ID NO:37). This may be repeated using any of the anti-connexin 26 peptides described herein (e.g., SRPTEKT (SEQ ID NO:38), or SRPTEKTVFTV (SEQ ID NO:37)) or any of the anti-Cx26 polynucleotides described herein. (e.g., 5′ TGT ATT GGG ACA AGG CCA GG 3′ (connexin 26) (SEQ. ID. NO:1),

(SEQ. ID. NO: 2)
5′ ATC TCT TCG ATG TCC TTA AA 3′ (connexin 26),
and/or
(connexin 26) (SEQ. ID. NO: 3))
5′ TCC TGA GCA ATA CCT AAC GAA CAA ATA 3′.

Wound biopsies are taken after 10 days and the tensile strength of the wounds is determined using an Instron tensiometer in accordance with the manufacturer's instructions and standardized to the cross sectional area of the wounds.

Subsequently, the quotient (E/C value) is calculated from the absolute value of the tensile strength of a wound that is treated and the absolute value of the tensile strength of a wound in the same animal that only receives the control preparation. The mean of the E/C values is determined and the changes in tensile strength relative to the treatment are determined.

### Example 10

### Chronic Wound Treatment

The method and compositions disclosed herein can be used to treat a human subject with a chronic wound (e.g., a diabetic or vasculitic ulcer or a persistent epithelial defect). A human subject with diabetes, or underlying peripheral vascular or arterial disease, first presents for complications arising from a non-closing or slow-healing foot or leg wound. The wound is treated with 2 mL of 20 CpM preparation of an anti-Cx26 polynucleotide according to the invention in pluronic gel (e.g., total dose ˜200-400 μg) based on a wound size of approximately 7 cm×5 cm (about 35 cm²) with a depth of approximately 3-4 mm (other appropriate dosages to be administered can be readily determined by a skilled practitioner in accordance with the wound size).

The wound site is dressed and covered for a period of 7 days. The wound is uncovered on Day 7 and the wound healing results are assessed.

The treatment outlined above is repeated (in appropriate dosage) as necessary or desired. Following this treatment, wound closure and wound appearance are assessed. Time to closure is also assessed. The patient is evaluated again at two weeks, one month, and/or two months and the wound (reduction if any) is again evaluated.

### Example 11

### Chronic Ulcer Treatment

The method and compositions disclosed herein are used to treat a human subject with a chronic venous leg ulceration.

Human test subjects are grouped according to ulcer size and minimum and maximum ulcer areas (e.g., 2 cm² and 50 cm²). Patient's resting ankle brachial doppler arterial pressure index are determined as a baseline (e.g., equal to or greater than 0.80).

All patients receive compression bandaging. The area of the ulcer is determined by tracing, and suitable dosages of an anti-Cx26 polynucleotide according to the invention in pluronic gel (e.g., total dose ˜200-400 μg) based on the size of the particular wound. As will be appreciated, appropriate dosages to be administered can be readily determined by a skilled practitioner in accordance with the wound size.

The patient is asked to lie recumbent on the examination couch while the test preparation is applied to the surface of the wound, and to remain there for 15 minutes before compression bandaging is applied.

The wound is uncovered on Day 7 and the wound healing results are assessed. Wound fluids and blood samples are analyzed for relevant wound healing biomarkers using bioassays.

The treatment outlined above is repeated (in appropriate dosage). Following this treatment, wound closure and wound appearance are assessed, as is time to healing. This course is repeated weekly or bi-weekly, or as appropriate given the state of healing, until wound closure.

### Example 12

### Diabetic Ulcer Treatment

The following are used to evaluate therapeutic efficacy of sequential administration of exemplary preparations in accelerating the healing rate of diabetic and other chronic ulcers.

The primary efficacy endpoint is the percentage of patients achieving full wound closure within 12-20 weeks. Secondary endpoints include the time to 100% closure, time to 80% closure, time to 50% closure, and the amount of wound closure as a percentage change from the baseline wound size at 3, 5, 10, 15, and 20 weeks. Kaplan-Meier survival analysis techniques are utilized to examine the time-to-event endpoints.

All patients receive a regimen of standard diabetic (or other) ulcer care consisting of initial sharp debridement, wound cleansing, wound dressing, and wound pressure offloading. The ulcer is optionally treated by wound cleansing, or by an initial sharp debridement and wound cleansing. Wound cleansing alone is preferred. A desired amount of an anti-Cx26 polynucleotide in a pluronic gel preparation is administered. In therapeutic regimens where a second anti-connexin agent is administered (e.g., substantially simultaneously (e.g., within about one minute or less of administration anti-Cx26 polynucleotide composition), the compositions are administered within 1 minute, 10 minutes, 30 minutes, 1 hour, 6 hours, 12 hours, or 24 hours of each other. The wound is dressed with a non-stick bandage and pressure bandage.

Wounds are evaluated twice a week for up to 12-20 weeks or until wound closure, whichever is earlier. Patients are removed from the study if they developed a clinical infection or if the wound condition significantly deteriorated. At each wound evaluation (twice weekly), the wound perimeter is traced for determination of wound area, and the wound is photographed with a digital camera. Blood chemistry and hematology tests are performed at patient enrollment, and at weeks 5, 10, 15, and 20. A radiographic assessment may be conducted every 5 weeks to study effects on underlying bone composition.

### Example 13

### Formulation of Anti-Connexin Agent

Anti-connexin agent is conveniently formulated in a form suitable for administration according to the methods of the present invention.

Suitable formulations include a mixture of the following formulating agents: polyquaternium 10, HEC (hydroxyethylcellulose), HPMC (hydroxypropylmethylcellulos), CMC (carboxymethylcellulose), sodium hyaluronate, Tween 20, Poloxamer 188, Pluronic 87 NF, cocamidopropyl betaine, sodium laureth sulfate, poly L-lysine, polyethylene imine, benzalkonium chloride, methyl paraben, propyl paraben, propylene glycol, and 10 mM phosphate buffer. The amount of the individual anti-connexin agent or agents and formulating agents can depend of the particular use intended.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A sustained release drug delivery device for delivering a plurality of connexin gap junction or hemichannel modulators to a desired location of the skin, comprising:
(a) a scaffold constructed of a biodegradable material; and
(b) a plurality of polymer coatings on the scaffold
(c) each coating comprising one or a plurality of connexin gap junction or hemichannel modulators and one or a plurality of biodegradable polymers,
wherein said plurality of gap junction modulators comprises at least a connexin 26 and a connexin 43 gap junction or hemichannel modulator, and wherein said coating has the characteristic of releasing said modulators into the skin tissue.

2. The sustained release drug delivery device of claim 1, wherein at least one of the gap junction modulators is a connexin modulator.

3. The sustained release drug delivery device of claim 2, wherein the connexin modulator is an antisense polynucleotide.

4. The sustained release drug delivery device of claim 3, wherein the antisense connexin polynucleotide is antisense to connexin 26 and/or connexin 43.

5. The sustained release drug delivery device of claim 1, wherein the scaffold biodegradable material comprises a connective tissue.

6. The sustained release drug delivery device of claim 5, wherein the connective tissue is selected from the group consisting of collagen, elastin, and chondroitin-4-sulfate.

7. The sustained release drug delivery device of claim 6, wherein the connective tissue is collagen.

8. The sustained release drug delivery device of claim 7, wherein the collagen is bovine collagen.

9. The sustained release drug delivery device of claim 5, wherein the scaffold biodegradable material comprises a connective tissue blended with a biodegradable polymer.

10. The sustained release drug delivery device of claim 1, wherein the biodegradable material comprises alginate.

11. The sustained release drug delivery device of claim 1, wherein the biodegradable polymer is a biodegradable polyester polymer.

12. The sustained release drug delivery device of claim 11, wherein the biodegradable polyester polymer is selected from: poly(L-lactide), poly(glycolide), poly(DL-lactide), poly(dioxanone), poly(DL-lactide-co-L-lactide), poly(DL-lactide-co-glycolide), poly(glycolide-co-trimethylene carbonate), poly(caprolactone) (“polycaprolactone”), poly(lactic-co-glycolic acid) (PLGA), poly(dioxanone) poly(glycolide-co-trimethylene carbonate), and mixtures thereof.

13. The sustained release drug delivery device of claim 12, wherein the biodegradable polyester polymer is polycaprolactone.

14. The sustained release drug delivery device of claim 12, wherein the biodegradable polyester polymer is poly(lactic-co-glycolic acid (PLGA).

15. The sustained release drug delivery device of claim 1, wherein the scaffold is formed from electrospinning a precursor material.

16. The sustained release drug delivery device of claim 15, wherein the precursor material is a connective tissue.

17. The sustained release drug delivery device of claim 15, wherein the scaffold is formed from electrospun fibers.

18. The sustained release drug delivery device of claim 17, wherein the electrospun fibers are further formed into a sheet.

19. The sustained release drug delivery device of claim 18, wherein the shape of the scaffold is extracted from the sheet.

20. The sustained release drug delivery device of claim 1, wherein the polymer coating is applied to the scaffold by a method selected from: dip-coating, spray-drying, and spin-casting.

21. The sustained release drug delivery device of claim 1, wherein the plurality of polymer coatings is two, three, or four polymer coatings.

22. The composition of claim 21, wherein the polymer coatings further comprise two coatings of polycaprolactone blended with a gap junction modulator and two coatings of PLGA blended with a gap junction modulator.

23. The sustained release drug delivery device of claim 22, wherein the polycaprolactone and PLGA polymer coatings are alternating.

24. The sustained release drug delivery device of claim 22, wherein the gap junction modulator in the polycaprolactone polymer coating is the same type as the gap junction modulator in the PLGA polymer coating.

25. The sustained release drug delivery device of claim 22, wherein the gap junction modulator in the polycaprolactone polymer coating is a different type from the gap junction modulator in the PLGA polymer coating.

26. The sustained release drug delivery device of claim 4, wherein the antisense connexin polynucleotide comprises a pharmaceutically acceptable carrier and a synthetic anti-connexin 26 polynucleotide not more than about 50 nucleotide residues in length that targets an accessible site in a connexin 26 messenger RNA under physiological conditions.

27. The sustained release drug delivery device of claim 4, wherein the antisense connexin polynucleotide comprises a pharmaceutically acceptable carrier and a synthetic anti-connexin 43 polynucleotide not more than about 50 nucleotide residues in length that targets an accessible site in a connexin 43 messenger RNA under physiological conditions.

28. The sustained release drug delivery device of claim 4, wherein the antisense connexin polynucleotide comprises a synthetic anti-connexin 26 polynucleotide and a synthetic anti-connexin 43 polynucleotide or a mixture comprising both, that further comprises at least about 10, 15, 20, 25, 30, 35, 40, 45, 50, 55, 60, 65, 70, 75, 80, 85, 90, or 95 nucleotide residues.

29. The sustained release drug delivery device of claim 4, wherein the antisense connexin polynucleotide comprises a synthetic anti-connexin 26 polynucleotide and/or a synthetic anti-connexin 43 polynucleotide or a mixture further comprising both, that further comprises an unmodified phosphodiester backbone.

30. The sustained release drug delivery device of claim 4, wherein the antisense connexin polynucleotide comprises a synthetic anti-connexin 26 or anti-connexin 43 polynucleotide or a mixture further comprising both, that further comprises a modified phosphodiester backbone, optionally a backbone that comprises one or more internucleotide linkages selected from the group consisting of phosphorothioate, methylphosphorate, and locked nucleic acid linkages.

31. The sustained release drug delivery device of claim 4, wherein the antisense connexin polynucleotide comprises a nucleotide sequence selected from the group consisting of:
TGTATTGGGACAAGGCCAGG (SEQ ID NO: 1), or ATCTCTTCGATGTCCTTAAA (SEQ ID NO: 2),
or a nucleotide sequence that has at least about 65%, 70%, 75%, 80%, 85%, 90%, 91%, 92%, 93%, 94%, 95%, 96%, 97%, 98%, or 99% sequence identity with a nucleotide of SEQ ID NO: 1 and/or a nucleotide sequence that has at least about 65%, 70%, 75%, 80%, 85%, 90%, 91%, 92%, 93%, 94%, 95%, 96%, 97%, 98%, or 99% sequence identity with a nucleotide of SEQ ID NO:2.

32. A sustained release drug delivery device comprising a synthetic anti-Cx26 polynucleotide according to claim 4, wherein the accessible site targeted by the polynucleotide comprises a nucleotide sequence selected from the group consisting of:
(SEQ. ID. NO: 153)
ACTCCACCAGCATTGGAAAG,
or
(SEQ. ID. NO: 154)
GACATTCAGCAGGATGCAAA,
or a nucleotide sequence that has at least about 65%, 70%, 75%, 80%, 85%, 90%, 91%, 92%, 93%, 94%, 95%, 96%, 97%, 98%, or 99% sequence identity with a nucleotide of SEQ ID NO:155 and/or a nucleotide sequence that has at least about 65%, 70%, 75%, 80%, 85%, 90%, 91%, 92%, 93%, 94%, 95%, 96%, 97%, 98%, or 99% sequence identity with a nucleotide of SEQ ID NO:154.

33. A sustained release drug delivery device comprising a synthetic anti-connexin 26 polynucleotide according to claim 4 that is selected from the group consisting of an antisense polynucleotide, a micro RNA, a small nuclear RNA (snRANA), an interfering RNA (RNAi), and a small interfering RNA (siRNA) in an amount effective to stimulate healing of a chronic wound.

34. A sustained release drug delivery device comprising an anti-Cx26 polynucleotide according to claim 4 and a carrier, optionally a pharmaceutically acceptable carrier, wherein the composition optionally comprises from about 0.1 to about 50,000 micrograms of the anti-Cx26 polynucleotide.

35. A sustained release drug delivery device according to claim 34 in a form suitable for subcutaneous or topical application.

39. A method to promote wound healing, comprising administering to a subject having a wound a sustained release drug delivery device according to claim 4 comprising a combination of an anti-Cx26 polynucleotide and an anti-Cx43 polynucleotide effective to promote wound healing, wherein the subject is a mammal selected from the group consisting of a human, a domestic animal, a farm animal, a zoo animal, a sports animal, and a pet.

40. A method according to claim 39, wherein the wound is a chronic wound, optionally a diabetic ulcer, a venous ulcer, a pressure ulcer, a vasculitic ulcer, or an arterial ulcer.

41. A method according to claim 39, that further comprises administering to the subject a topical therapeutic agent.

42. A method of treating a wound comprising administering to a subject in need thereof a sustained release drug delivery device according to claim 1, wherein the wound is a burn.

43. A method according to claim 42, that comprises administering a sustained release drug delivery device comprising a therapeutically effective amounts of said anti-connexin agent, wherein administration of said sustained release drug delivery device is carried out more than once.

44. A method according to claim 43, wherein the sustained release drug delivery device is administered about once a week.

45. A method according to claim 43, wherein the sustained release drug delivery device is administered about twice a week.

46. The method of any of claims 39 to 45, wherein the epidermal wound edge is reduced.

47. The method of any of claims 39 to 45, wherein the granulation tissue area is reduced.

48. The method of any of claims 39 to 45, wherein the concentration of myofibroblasts in the granulation tissue area are reduced.

49. An article of manufacture comprising package material containing a sustained release drug delivery device according to claim 1, together with instructions for use in or on a subject in order to promote or improve wound healing or tissue repair.

50. The article of manufacture according to claim 49, wherein the wound is a chronic wound.


SEQUENCE LISTING (210 sequences)

SEQ ID NO: 1 - DNA, 20 bp, Artificial Sequence
  Description of Artificial Sequence Synthetic oligonucleotide
tgtattgggacaaggccagg

SEQ ID NO: 2 - DNA, 20 bp, Artificial Sequence
  Description of Artificial Sequence Synthetic oligonucleotide
atctcttcgatgtccttaaa

SEQ ID NO: 3 - DNA, 27 bp, Artificial Sequence
  Description of Artificial Sequence Synthetic oligonucleotide
tcctgagcaatacctaacgaacaaata

SEQ ID NO: 4 - DNA, 30 bp, Artificial Sequence
  Description of Artificial Sequence Synthetic oligonucleotide
gtaattgcggcaagaagaattgtttctgtc

SEQ ID NO: 5 - DNA, 30 bp, Artificial Sequence
  Description of Artificial Sequence Synthetic oligonucleotide
gtaattgcggcaggaggaattgtttctgtc

SEQ ID NO: 6 - DNA, 30 bp, Artificial Sequence
  Description of Artificial Sequence Synthetic oligonucleotide
ggcaagagacaccaaagacactaccagcat

SEQ ID NO: 7 - DNA, 20 bp, Artificial Sequence
  Description of Artificial Sequence Synthetic oligonucleotide
catctccttggtgctcaacc

SEQ ID NO: 8 - DNA, 20 bp, Artificial Sequence
  Description of Artificial Sequence Synthetic oligonucleotide
ctgaagtcgacttggcttgg

SEQ ID NO: 9 - DNA, 21 bp, Artificial Sequence
  Description of Artificial Sequence Synthetic oligonucleotide
ctcagatagtggccagaatgc

SEQ ID NO: 10 - DNA, 20 bp, Artificial Sequence
  Description of Artificial Sequence Synthetic oligonucleotide
ttgtccaggtgactccaagg

SEQ ID NO: 11 - DNA, 25 bp, Artificial Sequence
  Description of Artificial Sequence Synthetic oligonucleotide
cgtccgagcccagaaagatgaggtc

SEQ ID NO: 12 - DNA, 19 bp, Artificial Sequence
  Description of Artificial Sequence Synthetic oligonucleotide
agaggcgcacgtgagacac

SEQ ID NO: 13 - DNA, 19 bp, Artificial Sequence
  Description of Artificial Sequence Synthetic oligonucleotide
tgaagacaatgaagatgtt

SEQ ID NO: 14 - DNA, 25 bp, Artificial Sequence
  Description of Artificial Sequence Synthetic oligonucleotide
tttcttttctatgtgctgttggtga

SEQ ID NO: 15 - protein, 226 aa, Unknown
  Description of Unknown Connexin 26 sequence
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 16 - protein, 396 aa, Unknown
  Description of Unknown Connexin 45 sequence
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDNEEDPMMYPEMELESDKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKVGSKAGSNKSTASSKSGDGKNSVWI

SEQ ID NO: 17 - protein, 382 aa, Unknown
  Description of Unknown Connexin 43 sequence
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 18 - protein, 11 aa, Artificial Sequence
  Description of Artificial Sequence Synthetic peptide
FEVAFLLIQWI

SEQ ID NO: 19 - protein, 11 aa, Artificial Sequence
  Description of Artificial Sequence Synthetic peptide
LLIQWYIGFSL

SEQ ID NO: 20 - protein, 16 aa, Artificial Sequence
  Description of Artificial Sequence Synthetic peptide
SLSAVYTCKRDPCPHQ

SEQ ID NO: 21 - protein, 12 aa, Artificial Sequence
  Description of Artificial Sequence Synthetic peptide
VDCFLSRPTEKT

SEQ ID NO: 22 - protein, 11 aa, Artificial Sequence
  Description of Artificial Sequence Synthetic peptide
SRPTEKTIFII

SEQ ID NO: 23 - protein, 13 aa, Artificial Sequence
  Description of Artificial Sequence Synthetic peptide
LGTAVESAWGDEQ

SEQ ID NO: 24 - protein, 12 aa, Artificial Sequence
  Description of Artificial Sequence Synthetic peptide
QSAFRCNTQQPG

SEQ ID NO: 25 - protein, 12 aa, Artificial Sequence
  Description of Artificial Sequence Synthetic peptide
QQPGCENVCYDK

SEQ ID NO: 26 - protein, 13 aa, Artificial Sequence
  Description of Artificial Sequence Synthetic peptide
VCYDKSFPISHVR

SEQ ID NO: 27 - protein, 38 aa, Artificial Sequence
  Description of Artificial Sequence Synthetic polypeptide
AAESVWGDEIKSSFICNTLQPGCNSVCYDHFFPISHVR

SEQ ID NO: 28 - protein, 12 aa, Artificial Sequence
  Description of Artificial Sequence Synthetic peptide
ESVWGDEKSSFI

SEQ ID NO: 29 - protein, 12 aa, Artificial Sequence
  Description of Artificial Sequence Synthetic peptide
ICNTLQPGCNSV

SEQ ID NO: 30 - protein, 12 aa, Artificial Sequence
  Description of Artificial Sequence Synthetic peptide
SVCYDHFFPISH

SEQ ID NO: 31 - protein, 25 aa, Artificial Sequence
  Description of Artificial Sequence Synthetic peptide
RLVKCEAFPCPNTVDCFVSRPTEKT

SEQ ID NO: 32 - protein, 12 aa, Artificial Sequence
  Description of Artificial Sequence Synthetic peptide
VKCEAFPCPNTV

SEQ ID NO: 33 - protein, 12 aa, Artificial Sequence
  Description of Artificial Sequence Synthetic peptide
VDCFVSRPTEKT

SEQ ID NO: 34 - protein, 13 aa, Artificial Sequence
  Description of Artificial Sequence Synthetic peptide
VCYDHFFPISHVR

SEQ ID NO: 35 - protein, 18 aa, Artificial Sequence
  Description of Artificial Sequence Synthetic peptide
VWGDEKSSFICNTLQPGY

SEQ ID NO: 36 - protein, 15 aa, Artificial Sequence
  Description of Artificial Sequence Synthetic peptide
DEKSSFICNTLQPGY

SEQ ID NO: 37 - protein, 11 aa, Artificial Sequence
  Description of Artificial Sequence Synthetic peptide
SRPTEKTVFTV

SEQ ID NO: 38 - protein, 7 aa, Artificial Sequence
  Description of Artificial Sequence Synthetic peptide
SRPTEKT

SEQ ID NO: 39 - protein, 12 aa, Artificial Sequence
  Description of Artificial Sequence Synthetic peptide
ICNTLQPGCNSV

SEQ ID NO: 40 - protein, 16 aa, Artificial Sequence
  Description of Artificial Sequence Synthetic peptide
FLDTLHVCRRSPCPHP

SEQ ID NO: 41 - protein, 18 aa, Artificial Sequence
  Description of Artificial Sequence Synthetic peptide
KRDPCHQVDCFLSRPTEK

SEQ ID NO: 42 - protein, 35 aa, Homo sapiens
KEVWGDEQADFVCNTLQPGCKNVCYDHYFPISHIR

SEQ ID NO: 43 - protein, 35 aa, Homo sapiens
QEVWGDEQEDFVCNTLQPGCKNVCYDHFFPVSHIR

SEQ ID NO: 44 - protein, 35 aa, Homo sapiens
EEVWDDEQKDFVCNTKQPGCPNVCYDEFFPVSHVR

SEQ ID NO: 45 - protein, 35 aa, Homo sapiens
ERVWGDEQKDFDCNTKQPGCTNVCYDNYFPISNIR

SEQ ID NO: 46 - protein, 35 aa, Homo sapiens
ERVWSDDHKDFDCNTRQPGCSNVCFDEFFPVSHVR

SEQ ID NO: 47 - protein, 35 aa, Homo sapiens
ESVWGDEKSSFICNTLQPGCNSVCYDQFFPISHVR

SEQ ID NO: 48 - protein, 35 aa, Homo sapiens
ESVWGDEQSDFECNTAQPGCTNVCYDQAFPISHIR

SEQ ID NO: 49 - protein, 35 aa, Homo sapiens; identical to SEQ ID NO: 48

SEQ ID NO: 50 - protein, 35 aa, Homo sapiens
RPVYQDEQERFVCNTLQPGCANVCYDVFSPVSHLR

SEQ ID NO: 51 - protein, 35 aa, Homo sapiens
ESAWGDEQSAFRCNTQQPGCENVCYDKSFPISHVR

SEQ ID NO: 52 - protein, 35 aa, Homo sapiens
EDVWGDEQSDFTCNTQQPGCBNVCYBRAFPISHIR

SEQ ID NO: 53 - protein, 35 aa, Homo sapiens
EAIYSDEQAKFTCNTRQPGCDNVCYDAFAPLSHVR

SEQ ID NO: 54 - protein, 35 aa, Homo sapiens
ESSWGDEQADFRCDTIQPGCQNVCTDQAFPISHIR

SEQ ID NO: 55 - protein, 36 aa, Homo sapiens
GESIYYDEQSKFVCNTEQPGCENVCYDAFAPLSHVR

SEQ ID NO: 56 - protein, 39 aa, Homo sapiens
MYVFYVMYDGFSMQRLVKCNAWPCPNTVDCFVSRPTEKT

SEQ ID NO: 57 - protein, 39 aa, Homo sapiens
MYVFYFLYNGYHLPWVLKCGIDPCPNLVDCFISRPTEKT

SEQ ID NO: 58 - protein, 39 aa, Homo sapiens
LYIFHRLYKDYDMPRVVACSVEPCPHTVDCYISRPTEKK

SEQ ID NO: 59 - protein, 40 aa, Homo sapiens
LYLLHTLWHGFNMPRLVQCANVAPCPNIVDCYIARPTEKK

SEQ ID NO: 60 - protein, 39 aa, Homo sapiens
LYVFHSFYPKYILPPVVKCHADPCPNIVDCFISKPSEKN

SEQ ID NO: 61 - protein, 39 aa, Homo sapiens
MYVFYLLYPGYAMVRLVKCDVYPCPNTVDCFVSRPTEKT

SEQ ID NO: 62 - protein, 32 aa, Homo sapiens
LYGWTMEPVFVCQRAPCPYLVDCFVSRPTEKT

SEQ ID NO: 63 - protein, 32 aa, Homo sapiens; identical to SEQ ID NO: 62

SEQ ID NO: 64 - protein, 38 aa, Homo sapiens
GALHYFLFGFLAPKKFPCTRPPCTGVVDCYVSRPTSKS

SEQ ID NO: 65 - protein, 38 aa, Homo sapiens
LLIQWYIYGFSLSAVYTCKRDPCPHQVDCFLSRPTEKT

SEQ ID NO: 66 - protein, 38 aa, Homo sapiens
IAGQYFLYGFELKPLYRCDRWPCPNTVDCFISRPTEKT

SEQ ID NO: 67 - protein, 38 aa, Homo sapiens
LVGQYLLYGFEVRPFFPCSRQPCPHVVDCFVSRPTEKT

SEQ ID NO: 68 - protein, 38 aa, Homo sapiens
IVGQYFIYGIFLTTLHVCRRSPCPHPVNCYVSRPTEKN

SEQ ID NO: 69 - protein, 38 aa, Homo sapiens
LIGQYFLYGFQVHPFYVCSRLPCHPKIDCFISRPTEKT

SEQ ID NO: 70 - protein, 42 aa, Homo sapiens
LLIQWYIYGFSLSAVYTCKRDPCPHQVDCFLSRPTEKTIFII

SEQ ID NO: 71 - protein, 43 aa, Homo sapiens
MYVFYVMYDGFSMQRLVKCNAWPCPNTVDCFVSRPTEKTVFTV

SEQ ID NO: 72 - protein, 42 aa, Homo sapiens
YVFYFLYNGYHLPWVLKCGIDPCPNLVDCFISRPTEKTVFTI

SEQ ID NO: 73 - protein, 43 aa, Homo sapiens
LYIFHRLYKDYDMPRVVACSVEPCPHTVDCYISRPTEKKVFTY

SEQ ID NO: 74 - protein, 42 aa, Homo sapiens
LYLLHTLWHGFNMPRLVQCANVAPCPNIVDCYIARPTEKKTY

SEQ ID NO: 75 - protein, 43 aa, Homo sapiens
LYVFHSFYPKYILPPVVKCHADPCPNIVDCFISKPSEKNIFTL

SEQ ID NO: 76 - protein, 43 aa, Homo sapiens
MYVFYLLYPGYAMVRLVKCDVYPCPNTVDCFVSRPTEKTVFTV

SEQ ID NO: 77 - protein, 36 aa, Homo sapiens
LYGWTMEPVFVCQRAPCPYLVDCFVSRPTEKTIFII

SEQ ID NO: 78 - protein, 36 aa, Homo sapiens; identical to SEQ ID NO: 77

SEQ ID NO: 79 - protein, 42 aa, Homo sapiens
GALHYFLFGFLAPKKFPCTRPPCTGVVDCYVSRPTEKSLLML

SEQ ID NO: 80 - protein, 42 aa, Homo sapiens
IAGQYFLYGFELKPLYRCDRWPCPNTVDCFISRPTEKTIFII

SEQ ID NO: 81 - protein, 42 aa, Homo sapiens
LVGQYLLYGFEVRPFFPCSRQPCPHVVDCFVSRPTEKTVFLL

SEQ ID NO: 82 - protein, 41 aa, Homo sapiens
IVGQYFIYGIFLTTLHVCRRSPCPHPVNCYSRPTEKNVFIV

SEQ ID NO: 83 - protein, 42 aa, Homo sapiens
LIGQYFLYGFQVHPFYVCSRLPCHPKIDCFISRPTEKTIFLL

SEQ ID NO: 84 - protein, 45 aa, Artificial Sequence
  Description of Artificial Sequence Synthetic polypeptide
LGTAAESSWGDEQADFRCDTIQPGCQNVCTDQAFPISHIRFWVLQ

SEQ ID NO: 85 - protein, 14 aa, Artificial Sequence
  Description of Artificial Sequence Synthetic peptide
LGTAAESSWGDEQA

SEQ ID NO: 86 - protein, 13 aa, Artificial Sequence
  Description of Artificial Sequence Synthetic peptide
DEQADFRCDTIQP

SEQ ID NO: 87 - protein, 13 aa, Artificial Sequence
  Description of Artificial Sequence Synthetic peptide
TIQPGCQNVCTDQ

SEQ ID NO: 88 - protein, 13 aa, Artificial Sequence
  Description of Artificial Sequence Synthetic peptide
VCTDQAFPISHIR

SEQ ID NO: 89 - protein, 13 aa, Artificial Sequence
  Description of Artificial Sequence Synthetic peptide
AFPISHIRFWVLQ

SEQ ID NO: 90 - protein, 47 aa, Artificial Sequence
  Description of Artificial Sequence Synthetic polypeptide
MEVGFIVGQYFIYGIFLTTLHVCRRSPCPHPVNCYVSRPTEKNVFIV

SEQ ID NO: 91 - protein, 11 aa, Artificial Sequence
  Description of Artificial Sequence Synthetic peptide
MEVGFIVGQYF

SEQ ID NO: 92 - protein, 12 aa, Artificial Sequence
  Description of Artificial Sequence Synthetic peptide
IVGQYFIYGIFL

SEQ ID NO: 93 - protein, 14 aa, Artificial Sequence
  Description of Artificial Sequence Synthetic peptide
GIFLTTLHVCRRSP

SEQ ID NO: 94 - protein, 12 aa, Artificial Sequence
  Description of Artificial Sequence Synthetic peptide
RRSPCPHPVNCY

SEQ ID NO: 95 - protein, 12 aa, Artificial Sequence
  Description of Artificial Sequence Synthetic peptide
VNCYVSRPTEKN

SEQ ID NO: 96 - protein, 11 aa, Artificial Sequence
  Description of Artificial Sequence Synthetic peptide
SRPTEKNVFIV

SEQ ID NO: 97 - protein, 46 aa, Artificial Sequence
  Description of Artificial Sequence Synthetic polypeptide
LTAVGGESIYYDEQSKFVCNTEQPGCENVCYDAFAPLSHVRFWVFQ

SEQ ID NO: 98 - protein, 15 aa, Artificial Sequence
  Description of Artificial Sequence Synthetic peptide
LTAVGGESIYYDEQS

SEQ ID NO: 99 - protein, 13 aa, Artificial Sequence
  Description of Artificial Sequence Synthetic peptide
DEQSKFVCNTEQP

SEQ ID NO: 100 - protein, 13 aa, Artificial Sequence
  Description of Artificial Sequence Synthetic peptide
TEQPGCENVCYDA

SEQ ID NO: 101 - protein, 13 aa, Artificial Sequence
  Description of Artificial Sequence Synthetic peptide
VCYDAFAPLSHVR

SEQ ID NO: 102 - protein, 12 aa, Artificial Sequence
  Description of Artificial Sequence Synthetic peptide
APLSHVRFWVFQ

SEQ ID NO: 103 - protein, 47 aa, Artificial Sequence
  Description of Artificial Sequence Synthetic polypeptide
FEVGFLIGQYFLYGFQVHPFYVCSRLPCHPKIDCFISRPTEKTIFLL

SEQ ID NO: 104 - protein, 11 aa, Artificial Sequence
  Description of Artificial Sequence Synthetic peptide
FEVGFLIGQYF

SEQ ID NO: 105 - protein, 12 aa, Artificial Sequence
  Description of Artificial Sequence Synthetic peptide
LIGQYFLYGFQV

SEQ ID NO: 106 - protein, 14 aa, Artificial Sequence
  Description of Artificial Sequence Synthetic peptide
GFQVHPFYVCSRLP

SEQ ID NO: 107 - protein, 12 aa, Artificial Sequence
  Description of Artificial Sequence Synthetic peptide
SRLPCHPKIDCF

SEQ ID NO: 108 - protein, 12 aa, Artificial Sequence
  Description of Artificial Sequence Synthetic peptide
IDCFISRPTEKT

SEQ ID NO: 109 - protein, 11 aa, Artificial Sequence
  Description of Artificial Sequence Synthetic peptide
SRPTEKTIFLL

SEQ ID NO: 110 - protein, 11 aa, Artificial Sequence
  Description of Artificial Sequence Synthetic peptide
SRGGEKNVFIV

SEQ ID NO: 111 - protein, 10 aa, Artificial Sequence
  Description of Artificial Sequence Synthetic peptide
YVCSRLPCHP

SEQ ID NO: 112 - protein, 11 aa, Artificial Sequence
  Description of Artificial Sequence Synthetic peptide
QVHPFYVCSRL

SEQ ID NO: 113 - protein, 13 aa, Artificial Sequence
  Description of Artificial Sequence Synthetic peptide
FEVGFLIGQYFLY

SEQ ID NO: 114 - protein, 12 aa, Artificial Sequence
  Description of Artificial Sequence Synthetic peptide
GQYFLYGFQVHP

SEQ ID NO: 115 - protein, 12 aa, Artificial Sequence
  Description of Artificial Sequence Synthetic peptide
GFQVHPFYVCSR

SEQ ID NO: 116 - protein, 12 aa, Artificial Sequence
  Description of Artificial Sequence Synthetic peptide
AVGGESIYYDEQ

SEQ ID NO: 117 - protein, 12 aa, Artificial Sequence
  Description of Artificial Sequence Synthetic peptide
YDEQSKFVCNTE

SEQ ID NO: 118 - protein, 12 aa, Artificial Sequence
  Description of Artificial Sequence Synthetic peptide
NTEQPGCENVCY

SEQ ID NO: 119 - protein, 12 aa, Artificial Sequence
  Description of Artificial Sequence Synthetic peptide
CYDAFAPLSHVR

SEQ ID NO: 120 - protein, 12 aa, Artificial Sequence
  Description of Artificial Sequence Synthetic peptide
FAPLSHVRFWVF

SEQ ID NO: 121 - protein, 5 aa, Artificial Sequence
  Description of Artificial Sequence Synthetic peptide
LIGQY

SEQ ID NO: 122 - protein, 5 aa, Artificial Sequence
  Description of Artificial Sequence Synthetic peptide
QVHPF

SEQ ID NO: 123 - protein, 5 aa, Artificial Sequence
  Description of Artificial Sequence Synthetic peptide
YVCSR

SEQ ID NO: 124 - protein, 5 aa, Artificial Sequence
  Description of Artificial Sequence Synthetic peptide
SRLPC

SEQ ID NO: 125 - protein, 5 aa, Artificial Sequence
  Description of Artificial Sequence Synthetic peptide
LPCHP

SEQ ID NO: 126 - protein, 5 aa, Artificial Sequence
  Description of Artificial Sequence Synthetic peptide
GESIY

SEQ ID NO: 127 - protein, 6 aa, Artificial Sequence
  Description of Artificial Sequence Synthetic peptide
YDEQSK

SEQ ID NO: 128 - protein, 6 aa, Artificial Sequence
  Description of Artificial Sequence Synthetic peptide
SKFVCN

SEQ ID NO: 129 - protein, 8 aa, Artificial Sequence
  Description of Artificial Sequence Synthetic peptide
TEQPGCEN

SEQ ID NO: 130 - protein, 8 aa, Artificial Sequence
  Description of Artificial Sequence Synthetic peptide
VCYDAFAP

SEQ ID NO: 131 - protein, 10 aa, Artificial Sequence
  Description of Artificial Sequence Synthetic peptide
LSHVRFWVFQ

SEQ ID NO: 132 - protein, 14 aa, Artificial Sequence
  Description of Artificial Sequence Synthetic peptide
LIQYFLYGFQVHPF

SEQ ID NO: 133 - protein, 13 aa, Artificial Sequence
  Description of Artificial Sequence Synthetic peptide
VHPFYCSRLPCHP

SEQ ID NO: 134 - protein, 22 aa, Artificial Sequence
  Description of Artificial Sequence Synthetic peptide
VGGESIYYDEQSKFVCNTEQPG

SEQ ID NO: 135 - protein, 22 aa, Artificial Sequence
  Description of Artificial Sequence Synthetic peptide
TEQPGCENVCYDAFAPLSHVRF

SEQ ID NO: 136 - protein, 14 aa, Artificial Sequence
  Description of Artificial Sequence Synthetic peptide
AFAPLSHVRFWVFQ

SEQ ID NO: 137 - protein, 9 aa, Artificial Sequence
  Description of Artificial Sequence Synthetic peptide
RPRPDDLEI

SEQ ID NO: 138 - protein, 20 aa, Artificial Sequence
  Description of Artificial Sequence Synthetic peptide
PSSRASSRASSRPRPDDLEI

SEQ ID NO: 139 - protein, 9 aa, Artificial Sequence
  Description of Artificial Sequence Synthetic peptide
RPRPDDLEV

SEQ ID NO: 140 - protein, 9 aa, Artificial Sequence
  Description of Artificial Sequence Synthetic peptide
CSGKSKKPV

SEQ ID NO: 141 - protein, 16 aa, Artificial Sequence
  Description of Artificial Sequence Synthetic peptide
CYLLIRYCSGKSKKPV

SEQ ID NO: 142 - protein, 16 aa, Artificial Sequence
  Description of Artificial Sequence Synthetic peptide
RQPKIWFPNRRKPWKK

SEQ ID NO: 143 - protein, 31 aa, Artificial Sequence
  Description of Artificial Sequence Synthetic polypeptide
NSKKLAAGHELQPLAIVDQRPSSRASSRASS

SEQ ID NO: 144 - protein, 25 aa, Artificial Sequence
  Description of Artificial Sequence Synthetic peptide
RQPKIWFPNRRKPWKKRPRPDDLEI

SEQ ID NO: 145 - protein, 40 aa, Artificial Sequence
  Description of Artificial Sequence Synthetic polypeptide
NSKKLAAGHELQPLAIVDQRPSSRASSRASSRPRPDDLEI

SEQ ID NO: 146 - protein, 25 aa, Artificial Sequence
  Description of Artificial Sequence Synthetic peptide
RQPKIWFPNRRKPWKKCSGKSKKPV

SEQ ID NO: 147 - DNA, 1314 bp, Homo sapiens
ggcttttagcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 148 - DNA, 1149 bp, Homo sapiens
atgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 149 - DNA, 2347 bp, Homo sapiens
ggggtgcggttaaaaggcgccacggcgggagacaggtgttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataatttaaaaaaaaaaaaaaaaa

SEQ ID NO: 150 - DNA, 2445 bp, Rattus norvegicus
cgcggcgcgctccgctctcccaactcgcagccagtcggcggcgctcccgcctactgagcgcagcctccaccaggatccgcggggaccagctcgggatcagccggcgacccacttctgaccaacccaggagcggcccgatacccactcccgaccaacccgcgaccgacccagggacccactccggacctgctccttacaggggacagcgcctcgccgcttcccgccgcccagcgccgcacgctcctcgggacacagtgccaaccatccagaggacaagatggattggggcacactacagagcatcctcgggggtgtcaacaagcactccaccagcattgggaaaatctggctcactgtcctcttcatcttccgcatcatgatcctcgtggtggccgcgaaggaggtgtggggagatgagcaagccgattttgtttgcaacactctccagcctggctgtaagaatgtgtgctacgaccactacttccccatctctcacatccggctctgggctctgcagctgatcatggtgtccacgccggccctcctggtagctatgcacgtggcctaccggagacacgaaaagaaacggaagttcatgaagggagagataaagaacgagtttaaggacatcgaagagatcaaaacccagaaggtccgtatcgaagggtccctgtggtggacctacaccaccagcatcttcttccgggtcatcttcgaagctgtcttcatgtatgtcttttacatcatgtacaatggcttcttcatgcagcgtctggtgaagtgtaacgcctggccttgtcccaatacagtggactgcttcatttccaggcccacagaaaagactgtcttcacggtgttcatgatctctgtgtctggaatttgcatcctgctaaacatcacagagctgtgctatctgttcattaggtattgctcagggaagtccaaaagaccagtctaatgcattgcctggctgttaagcaaagatgagggagaggatgaggcaacctgtgcttagttatcagagttcagctaccagcatctcccgggcaaacattcccaccttaaatgccgccatttgaagtcccccgcaggcctcccatgaaactccagaagcctccatgggcctcccttcccccaaagctcccaaacaaaggcccaattctatgcctgtattaatgggttctaaagttagttagaccccgtgctggtgtgactatgctttaggatacattcacagtttaaacaaagggatctcacattgtttctcttcctctgaggacaggagacatgagcccagtcctgaggaaggtacagagaaagttccttcttccgggtccccttccccaagttgcccccagttaagggtaaagaatcttcgttctgttattttctttcatagtttaagtttgcaacaatggacaaaagctatttaatgtttcaagctagctgtgtcctttttttttttttaaatgaaaaccttaaaatgataggttcttttgttcttaaaatgatctggaaagcattatacattcctcctatttcagaggttcggtttgtgatgtgagcatggtgtataaccagatctcacaaggtctttaaaacgttggccttttggttatgggaaacctgggctgtggctgagagcccacctactgtattcatccttggtgtgctgagtacagcccgcaacaacgttacagcctgtctcaaatgagacaaactggaagcttctcgtgttagcttctgacaagaagaggccttgattaaaattttcaaccgtaattttgtgtaagaggcagataggttatgcctacaactgccccctgccatgagcctaactcagcccccctccacccccagctcgtctactctgtagctgtgggatgtggcagtcagtatcaaaagacttcatgagtttgcttgggaatttcactgccatggtacaatttaatggtgcagaaacaagatggggtggttttcaaagaaccgatgaaacttctagactctaaatcctgttgattaaaactgagtttttctactttgaatgtctgtttgcctcccttttcagcattgccttctaaactggaaacagaaatgttgatatttggaaaaaatagaagaaactagtttaggtcaatgtgtaacttttctaggacaagttgaaccttagcattgtcattctgcctgatgtgttgtccacaagatgacagtcaacaaatccaacaggggacacttcttcctgccaagaatgtcgttgggaagccattctgtaacaataaataagagttgtggtttcatgcttgtcactgatataattttgtaaactcctaaatgtcatttattaaaatatgattattttgtatactttaaaattaaataaggtttttaatagaataccaaactttaatgatcaatagtaaaaaaaaaaaaaaaaaaaaaaaaaaa

SEQ ID NO: 151 - DNA, 20 bp, Artificial Sequence
  Description of Artificial Sequence Synthetic primer
atgagcagtctgcctttcgt

SEQ ID NO: 152 - DNA, 20 bp, Artificial Sequence
  Description of Artificial Sequence Synthetic primer
gggtcgctctttcccttaac

SEQ ID NO: 153 - DNA, 20 bp, Artificial Sequence
  Description of Artificial Sequence Synthetic primer
actccaccagcattggaaag

SEQ ID NO: 154 - DNA, 20 bp, Artificial Sequence
  Description of Artificial Sequence Synthetic primer
gacattcagcaggatgcaaa

SEQ ID NO: 155 - DNA, 20 bp, Artificial Sequence
  Description of Artificial Sequence Synthetic primer
tgcaaacggggctgacctcc

SEQ ID NO: 156 - DNA, 20 bp, Artificial Sequence
  Description of Artificial Sequence Synthetic primer
tggggacgagctttattgag

SEQ ID NO: 157 - DNA, 20 bp, Artificial Sequence
  Description of Artificial Sequence Synthetic oligonucleotide
ccaggctgactcaaccgctg

SEQ ID NO: 158 - DNA, 20 bp, Artificial Sequence
  Description of Artificial Sequence Synthetic oligonucleotide
tctccccacacctcctttgc

SEQ ID NO: 159 - DNA, 20 bp, Homo sapiens
gcaaaggaggtgtggggaga

SEQ ID NO: 160 - DNA, 20 bp, Artificial Sequence
  Description of Artificial Sequence Synthetic oligonucleotide
cttcttctcatgtctccggt

SEQ ID NO: 161 - DNA, 20 bp, Homo sapiens
accggagacatgagaagaag

SEQ ID NO: 162 - DNA, 20 bp, Artificial Sequence
  Description of Artificial Sequence Synthetic oligonucleotide
tcactctttatctccccctt

SEQ ID NO: 163 - DNA, 20 bp, Homo sapiens
aagggggagataaagagtga

SEQ ID NO: 164 - DNA, 20 bp, Artificial Sequence
  Description of Artificial Sequence Synthetic oligonucleotide
tgggttttgatctcctcgat

SEQ ID NO: 165 - DNA, 20 bp, Homo sapiens
atcgaggagatcaaaaccca

SEQ ID NO: 166 - DNA, 20 bp, Artificial Sequence
  Description of Artificial Sequence Synthetic oligonucleotide
tcgatgcggaccttctgggt

SEQ ID NO: 167 - DNA, 20 bp, Homo sapiens
acccagaaggtccgcatcga

SEQ ID NO: 168 - DNA, 20 bp, Artificial Sequence
  Description of Artificial Sequence Synthetic oligonucleotide
cccagaacaatatctaatta

SEQ ID NO: 169 - DNA, 20 bp, Homo sapiens
taattagatattgttctggg

SEQ ID NO: 170 - DNA, 20 bp, Artificial Sequence
  Description of Artificial Sequence Synthetic oligonucleotide
gcttttttgacttcccagaa

SEQ ID NO: 171 - DNA, 20 bp, Homo sapiens
ttctgggaagtcaaaaaagc

SEQ ID NO: 172 - DNA, 18 bp, Artificial Sequence
  Description of Artificial Sequence Synthetic primer
cgggagacaggtgttgcg

SEQ ID NO: 173 - DNA, 18 bp, Artificial Sequence
  Description of Artificial Sequence Synthetic primer
ctcctaggcgggtcctgg

SEQ ID NO: 174 - DNA, 24 bp, Artificial Sequence
  Description of Artificial Sequence Synthetic primer
acttttggtacattgtggcttcaa

SEQ ID NO: 175 - DNA, 20 bp, Artificial Sequence
  Description of Artificial Sequence Synthetic primer
ccgccaggacaaaccagtat

SEQ ID NO: 176 - DNA, 20 bp, Artificial Sequence
  Description of Artificial Sequence Synthetic oligonucleotide
cgtgccccaatccatcttct

SEQ ID NO: 177 - DNA, 20 bp, Homo sapiens
agaagatggattggggcacg

SEQ ID NO: 178 - DNA, 20 bp, Artificial Sequence
  Description of Artificial Sequence Synthetic oligonucleotide
catctccccacacctccttt

SEQ ID NO: 179 - DNA, 20 bp, Homo sapiens
aaaggaggtgtggggagatg

SEQ ID NO: 180 - DNA, 20 bp, Artificial Sequence
  Description of Artificial Sequence Synthetic oligonucleotide
tcgtagcacacgttcttgca

SEQ ID NO: 181 - DNA, 20 bp, Homo sapiens
tgcaagaacgtgtgctacga

SEQ ID NO: 182 - DNA, 20 bp, Artificial Sequence
  Description of Artificial Sequence Synthetic oligonucleotide
atgtgggagatggggaagta

SEQ ID NO: 183 - DNA, 20 bp, Homo sapiens
tacttccccatctcccacat

SEQ ID NO: 184 - DNA, 20 bp, Artificial Sequence
  Description of Artificial Sequence Synthetic oligonucleotide
cggtaggccacgtgcatggc

SEQ ID NO: 185 - DNA, 20 bp, Homo sapiens
gccatgcacgtggcctaccg

SEQ ID NO: 186 - DNA, 20 bp, Artificial Sequence
  Description of Artificial Sequence Synthetic oligonucleotide
atctcctcgatgtccttaaa

SEQ ID NO: 187 - DNA, 20 bp, Homo sapiens
tttaaggacatcgaggagat

SEQ ID NO: 188 - DNA, 20 bp, Artificial Sequence
  Description of Artificial Sequence Synthetic oligonucleotide
agtgttgggacaaggccagg

SEQ ID NO: 189 - DNA, 20 bp, Homo sapiens
cctggccttgtcccaacact

SEQ ID NO: 190 - DNA, 20 bp, Artificial Sequence
  Description of Artificial Sequence Synthetic oligonucleotide
caatcatgaacactgtgaag

SEQ ID NO: 191 - DNA, 20 bp, Homo sapiens
cttcacagtgttcatgattg

SEQ ID NO: 192 - DNA, 20 bp, Artificial Sequence
  Description of Artificial Sequence Synthetic oligonucleotide
aggatgcaaattccagacac

SEQ ID NO: 193 - DNA, 20 bp, Homo sapiens
gtgtctggaatttgcatcct

SEQ ID NO: 194 - DNA, 20 bp, Artificial Sequence
  Description of Artificial Sequence Synthetic oligonucleotide
tcgatacggaccttctgggt

SEQ ID NO: 195 - DNA, 20 bp, Rattus norvegicus
acccagaaggtccgtatcga

SEQ ID NO: 196 - DNA, 20 bp, Artificial Sequence
  Description of Artificial Sequence Synthetic oligonucleotide
tcgtagcacacattcttaca

SEQ ID NO: 197 - DNA, 20 bp, Rattus norvegicus
tgtaagaatgtgtgctacga

SEQ ID NO: 198 - DNA, 20 bp, Rattus norvegicus
cctggccttgtcccaataca

SEQ ID NO: 199 - DNA, 20 bp, Artificial Sequence
  Description of Artificial Sequence Synthetic oligonucleotide
tgtgccccaatccatcttgt

SEQ ID NO: 200 - DNA, 20 bp, Rattus norvegicus
acaagatggattggggcaca

SEQ ID NO: 201 - DNA, 20 bp, Artificial Sequence
  Description of Artificial Sequence Synthetic oligonucleotide
catctccccacacctccttc

SEQ ID NO: 202 - DNA, 20 bp, Rattus norvegicus
gaaggaggtgtggggagatg

SEQ ID NO: 203 - DNA, 20 bp, Rattus norvegicus
tttaaggacatcgaagagat

SEQ ID NO: 204 - DNA, 20 bp, Artificial Sequence
  Description of Artificial Sequence Synthetic oligonucleotide
atgtgagagatggggaagta

SEQ ID NO: 205 - DNA, 20 bp, Rattus norvegicus
tacttccccatctctcacat

SEQ ID NO: 206 - DNA, 20 bp, Artificial Sequence
  Description of Artificial Sequence Synthetic oligonucleotide
cggtaggccacgtgcatagc

SEQ ID NO: 207 - DNA, 20 bp, Rattus norvegicus
gctatgcacgtggcctaccg

SEQ ID NO: 208 - DNA, 20 bp, Artificial Sequence
  Description of Artificial Sequence Synthetic oligonucleotide
agatcatgaacaccgtgaag

SEQ ID NO: 209 - DNA, 20 bp, Rattus norvegicus
cttcacggtgttcatgatct

SEQ ID NO: 210 - DNA, 30 bp, Artificial Sequence
  Description of Artificial Sequence Synthetic oligonucleotide
gacagaaacaattcctcctgccgcaattac